SEQUENCE LISTING (38 sequences)

SEQ ID NO: 1 - DNA, 7650 bp, Homo sapiens
  gene 1..7650: note = wild type mTOR
  source 1..7650: mol_type = genomic DNA
atgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaa

SEQ ID NO: 2 - protein, 2549 aa, Homo sapiens
  PEPTIDE 1..2549: note = wild type mTOR
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 3 - DNA, 3495 bp, Homo sapiens
  gene 1..3495: note = wild type TSC1
  source 1..3495: mol_type = genomic DNA
atggcccaacaagcaaatgtcggggagcttcttgccatgctggactcccccatgctgggtgtgcgggacgacgtgacagctgtctttaaagagaacctcaattctgaccgtggccctatgcttgtaaacaccttggtggattattacctggaaaccagctctcagccggcattgcacatcctgaccaccttgcaagagccacatgacaagcacctcttggacaggattaacgaatatgtgggcaaagccgccactcgtttatccatcctctcgttactgggtcatgtcataagactgcagccatcttggaagcataagctctctcaagcacctcttttgccttctttactaaaatgtctcaagatggacactgacgtcgttgtcctcacaacaggcgtcttggtgttgataaccatgctaccaatgattccacagtctgggaaacagcatcttcttgatttctttgacatttttggccgtctgtcatcatggtgcctgaagaaaccaggccacgtggcggaagtctatctcgtccatctccatgccagtgtgtacgcactctttcatcgcctttatggaatgtacccttgcaacttcgtctcctttttgcgttctcattacagtatgaaagaaaacctggagacttttgaagaagtggtcaagccaatgatggagcatgtgcgaattcatccggaattagtgactggatccaaggaccatgaactggaccctcgaaggtggaagagattagaaactcatgatgttgtgatcgagtgtgccaaaatctctctggatcccacagaagcctcatatgaagatggctattctgtgtctcaccaaatctcagcccgctttcctcatcgttcagccgatgtcaccaccagcccttatgctgacacacagaatagctatgggtgtgctacttctaccccttactccacgtctcggctgatgttgttaaatatgccagggcagctacctcagactctgagttccccatcgacacggctgataactgaaccaccacaagctactctttggagcccatctatggtttgtggtatgaccactcctccaacttctcctggaaatgtcccacctgatctgtcacacccttacagtaaagtctttggtacaactgcaggtggaaaaggaactcctctgggaaccccagcaacctctcctcctccagccccactctgtcattcggatgactacgtgcacatttcactcccccaggccacagtcacaccccccaggaaggaagagagaatggattctgcaagaccatgtctacacagacaacaccatcttctgaatgacagaggatcagaagagccacctggcagcaaaggttctgtcactctaagtgatcttccagggtttttaggtgatctggcctctgaagaagatagtattgaaaaagataaagaagaagctgcaatatctagagaactttctgagatcaccacagcagaggcagagcctgtggttcctcgaggaggctttgactctcccttttaccgagacagtctcccaggttctcagcggaagacccactcggcagcctccagttctcagggcgccagcgtgaaccctgagcctttacactcctccctggacaagcttgggcctgacacaccaaagcaagcctttactcccatagacctgccctgcggcagtgctgatgaaagccctgcgggagacagggaatgccagacttctttggagaccagtatcttcactcccagtccttgtaaaattccacctccgacgagagtgggctttggaagcgggcagcctcccccgtatgatcatctttttgaggtggcattgccaaagacagcccatcattttgtcatcaggaagactgaggagctgttaaagaaagcaaaaggaaacacagaggaagatggtgtgccctctacctccccaatggaagtgctggacagactgatacagcagggagcagacgcgcacagcaaggagctgaacaagttgcctttacccagcaagtctgtcgactggacccactttggaggctctcctccttcagatgagatccgcaccctccgagaccagttgcttttactgcacaaccagttactctatgagcgttttaagaggcagcagcatgccctccggaacaggcggctcctccgcaaggtgatcaaagcagcagctctggaggaacataatgctgccatgaaagatcagttgaagttacaagagaaggacatccagatgtggaaggttagtctgcagaaagaacaagctagatacaatcagctccaggagcagcgtgacactatggtaaccaagctccacagccagatcagacagctgcagcatgaccgagaggaattctacaaccagagccaggaattacagacgaagctggaggactgcaggaacatgattgcggagctgcggatagaactgaagaaggccaacaacaaggtgtgtcacactgagctgctgctcagtcaggtttcccaaaagctctcaaacagtgagtcggtccagcagcagatggagttcttgaacaggcagctgttggttcttggggaggtcaacgagctctatttggaacaactgcagaacaagcactcagataccacaaaggaagtagaaatgatgaaagccgcctatcggaaagagctagaaaaaaacagaagccatgttctccagcagactcagaggcttgatacctcccaaaaacggattttggaactggaatctcacctggccaagaaagaccaccttcttttggaacagaagaaatatctagaggatgtcaaactccaggcaagaggacagctgcaggccgcagagagcaggtatgaggctcagaaaaggataacccaggtgtttgaattggagatcttagatttatatggcaggttggagaaagatggcctcctgaaaaaacttgaagaagaaaaagcagaagcagctgaagcagcagaagaaaggcttgactgttgtaatgacgggtgctcagattccatggtagggcacaatgaagaggcatctggccacaacggtgagaccaagacccccaggcccagcagcgcccggggcagtagtggaagcagaggtggtggaggcagcagcagcagcagcagcgagctttctaccccagagaaacccccacaccagagggcaggcccattcagcagtcggtgggagacgactatgggagaagcgtctgccagcatccccaccactgtgggctcacttcccagttcaaaaagcttcctgggtatgaaggctcgagagttatttcgtaataagagcgagagccagtgtgatgaggacggcatgaccagtagcctttctgagagcctaaagacagaactgggcaaagacttgggtgtggaagccaagattcccctgaacctagatggccctcacccgtctcccccgaccccggacagtgttggacagctacatatcatggactacaatgagactcatcatgaacacagctaa

SEQ ID NO: 4 - protein, 1164 aa, Homo sapiens
  PEPTIDE 1..1164: note = wild type TSC1
MAQQANVGELLAMLDSPMLGVRDDVTAVFKENLNSDRGPMLVNTLVDYYLETSSQPALHILTTLQEPHDKHLLDRINEYVGKAATRLSILSLLGHVIRLQPSWKHKLSQAPLLPSLLKCLKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSKDHELDPRRWKRLETHDVVIECAKISLDPTEASYEDGYSVSHQISARFPHRSADVTTSPYADTQNSYGCATSTPYSTSRLMLLNMPGQLPQTLSSPSTRLITEPPQATLWSPSMVCGMTTPPTSPGNVPPDLSHPYSKVFGTTAGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSIFTPSPCKIPPPTRVGFGSGQPPPYDHLFEVALPKTAHHFVIRKTEELLKKAKGNTEEDGVPSTSPMEVLDRLIQQGADAHSKELNKLPLPSKSVDWTHFGGSPPSDEIRTLRDQLLLLHNQLLYERFKRQQHALRNRRLLRKVIKAAALEEHNAAMKDQLKLQEKDIQMWKVSLQKEQARYNQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSARGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHPSPPTPDSVGQLHIMDYNETHHEHS

SEQ ID NO: 5 - DNA, 5424 bp, Homo sapiens
  gene 1..5424: note = wild type TSC2
  source 1..5424: mol_type = genomic DNA
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgactacatggagccagagagaggctctgagaagaagaccagcggccccctttctcctcccacagggcctcctggcccggcgcctgcaggccccgccgtgcggctggggtccgtgccctactccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtcttttgatgacacccccgagaaggacagcttcagggcccggagtactagtctcaacgagagacccaagagtctgaggatagccagaccccccaaacaaggcttgaataactctccacccgtgaaagaattcaaggagagctctgcagccgaggccttccggtgccgcagcatcagtgtgtctgaacatgtggtccgcagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtgtga

SEQ ID NO: 6 - protein, 1807 aa, Homo sapiens
  PEPTIDE 1..1807: note = wild type TSC2
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 7 - DNA, 1398 bp, Homo sapiens
  gene 1..1398: note = wild type AKT3
  source 1..1398: mol_type = genomic DNA
atgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatgtcagcaatcagattgtggcatgctgggtaactggaaaaaataa

SEQ ID NO: 8 - protein, 465 aa, Homo sapiens
  PEPTIDE 1..465: note = wild type AKT3
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKCQQSDCGMLGNWKK

SEQ ID NO: 9 - DNA, 3207 bp, Homo sapiens
  gene 1..3207: note = wild type PIK3CA
  source 1..3207: mol_type = genomic DNA
atgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactga

SEQ ID NO: 10 - protein, 1068 aa, Homo sapiens
  PEPTIDE 1..1068: note = wild type PIK3CA
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 11 - DNA, 20 bp, synthetic construct
  misc_feature 1..20: note = C1483 sense primer
  source 1..20: mol_type = other DNA
taggttacaggcctggatgg

SEQ ID NO: 12 - DNA, 20 bp, synthetic construct
  misc_feature 1..20: note = C1483 antisense primer
  source 1..20: mol_type = other DNA
cttggcctcccaaaatgtta

SEQ ID NO: 13 - DNA, 21 bp, synthetic construct
  misc_feature 1..21: note = L2427 sense primer
  source 1..21: mol_type = other DNA
tccaggctacctggtatgaga

SEQ ID NO: 14 - DNA, 20 bp, synthetic construct
  misc_feature 1..20: note = L2427 antisense primer
  source 1..20: mol_type = other DNA
gccttcctttcaaatccaaa

SEQ ID NO: 15 - DNA, 36 bp, synthetic construct
  misc_feature 1..36: note = annealing forward primer for pCIG-mTOR mutant-IRES-EGFP
  source 1..36: mol_type = other DNA
aattccaattgcccgggcttaagatcgatacgcgta

SEQ ID NO: 16 - DNA, 36 bp, synthetic construct
  misc_feature 1..36: note = annealing reverse primer for pCIG-mTOR mutant-IRES-EGFP
  source 1..36: mol_type = other DNA
ccggtacgcgtatcgatcttaagcccgggcaattgg

SEQ ID NO: 17 - DNA, 50 bp, synthetic construct
  misc_feature 1..50: note = forward primer for pCIG-mTOR mutant-IRES-EGFP
  source 1..50: mol_type = other DNA
gatcacaattgtggccaccatggactacaaggacgacgatgacaagatgc

SEQ ID NO: 18 - DNA, 41 bp, synthetic construct
  misc_feature 1..41: note = reverse primer for pCIG-mTOR mutant-IRES-EGFP
  source 1..41: mol_type = other DNA
tgatcaacgcgtttaccagaaagggcaccagccaatatagc

SEQ ID NO: 19 - DNA, 33 bp, synthetic construct
  misc_feature 1..33: note = Y1450D sense primer
  source 1..33: mol_type = other DNA
tcgtgcagtttctcatcccaggtagcctggatc

SEQ ID NO: 20 - DNA, 33 bp, synthetic construct
  misc_feature 1..33: note = Y1450D antisense primer
  source 1..33: mol_type = other DNA
gatccaggctacctgggatgagaaactgcacga

SEQ ID NO: 21 - DNA, 23 bp, synthetic construct
  misc_feature 1..23: note = C1483R sense primer
  source 1..23: mol_type = other DNA
ggcctcgaggcggcgcatgcggc

SEQ ID NO: 22 - DNA, 23 bp, synthetic construct
  misc_feature 1..23: note = C1483R antisense primer
  source 1..23: mol_type = other DNA
gccgcatgcgccgcctcgaggcc

SEQ ID NO: 23 - DNA, 33 bp, synthetic construct
  misc_feature 1..33: note = L2427Q sense primer
  source 1..33: mol_type = other DNA
gtctatgaccccttgcagaactggaggctgatg

SEQ ID NO: 24 - DNA, 33 bp, synthetic construct
  misc_feature 1..33: note = L2427Q antisense primer
  source 1..33: mol_type = other DNA
catcagcctccagttctgcaaggggtcatagac

SEQ ID NO: 25 - DNA, 33 bp, synthetic construct
  misc_feature 1..33: note = L2427P sense primer
  source 1..33: mol_type = other DNA
gtctatgaccccttgccgaactggaggctgatg

SEQ ID NO: 26 - DNA, 33 bp, synthetic construct
  misc_feature 1..33: note = L2427P antisense primer
  source 1..33: mol_type = other DNA
catcagcctccagttcggcaaggggtcatagac

SEQ ID NO: 27 - DNA, 21 bp, synthetic construct
  misc_feature 1..21: note = 7280T sense primer
  source 1..21: mol_type = other DNA
cccaggcacttgatgatactc

SEQ ID NO: 28 - DNA, 20 bp, synthetic construct
  misc_feature 1..20: note = 7280T antisense primer
  source 1..20: mol_type = other DNA
cttgctttgggtggagagtt

SEQ ID NO: 29 - DNA, 27 bp, synthetic construct
  misc_feature 1..27: note = TSC-1 R22W-F primer
  source 1..27: mol_type = other DNA
gtcacgtcgtcccacacacccagcatg

SEQ ID NO: 30 - DNA, 27 bp, synthetic construct
  misc_feature 1..27: note = TSC-1 R22W-R primer
  source 1..27: mol_type = other DNA
catgctgggtgtgtgggacgacgtgac

SEQ ID NO: 31 - DNA, 43 bp, synthetic construct
  misc_feature 1..43: note = TSC-1 R204C-F primer
  source 1..43: mol_type = other DNA
ctttcatactgtaatgagaacacaaaaaggagacgaagttgca

SEQ ID NO: 32 - DNA, 43 bp, synthetic construct
  misc_feature 1..43: note = TSC-1 R204C-R primer
  source 1..43: mol_type = other DNA
tgcaacttcgtctcctttttgtgttctcattacagtatgaaag

SEQ ID NO: 33 - DNA, 33 bp, synthetic construct
  misc_feature 1..33: note = TSC-2 V1547I-F primer
  source 1..33: mol_type = other DNA
tctccaacatacaggatggcgatcttgtgggtg

SEQ ID NO: 34 - DNA, 33 bp, synthetic construct
  misc_feature 1..33: note = TSC-2 V1547I-R primer
  source 1..33: mol_type = other DNA
cacccacaagatcgccatcctgtatgttggaga

SEQ ID NO: 35 - DNA, 35 bp, synthetic construct
  misc_feature 1..35: note = AKT3 R247H-F primer
  source 1..35: mol_type = other DNA
caccatagaaacgtgtgtggtcctcagagaacacc

SEQ ID NO: 36 - DNA, 35 bp, synthetic construct
  misc_feature 1..35: note = AKT3 R247H-R primer
  source 1..35: mol_type = other DNA
ggtgttctctgaggaccacacacgtttctatggtg

SEQ ID NO: 37 - DNA, 20 bp, synthetic construct
  misc_feature 1..20: note = DNA sequence corresponding to TSC1 targetting sgRNA
  source 1..20: mol_type = other DNA
tgctggactcctccacactg

SEQ ID NO: 38 - DNA, 20 bp, synthetic construct
  misc_feature 1..20: note = DNA sequence corresponding to TSC2 targetting sgRNA
  source 1..20: mol_type = other DNA
aatcccaggtgtgcagaagg

(19) United States
(12) Patent Application Publication
(10) Pub. No.: US 2026/0041692 A1
(43) Pub. Date: Feb. 12, 2026

(54) COMPOSITION FOR PREVENTION OR TREATMENT OF INTRACTABLE EPILEPSY COMPRISING mTOR INHIBITOR

(71) Applicant: SoVarGen Co., Ltd., Yuseong-gu (KR)

(72) Inventors: Jeong Ho LEE, Daejeon (KR); Jae Seok LIM, Daejeon (KR); Woo-Il KIM, Daejeon (KR); Dong Seok KIM, Seoul (KR); Hoon Chul KANG, Seoul (KR); Se Hoon KIM, Seoul (KR)

(21) Appl. No.: 19/346,109

(22) Filed: Sep. 30, 2025

Related U.S. Application Data
(63) Continuation of application No. 17/171,908, filed on Feb. 9, 2021, now Pat. No. 12,453,733.
Application No. 17/171,908 is a continuation of application No. 15/555,622, now abandoned.

(30) Foreign Application Priority Data
Mar. 6, 2015 (KR) 10-2015-0031606
Mar. 20, 2015 (KR) 10-2015-0038668
Jan. 29, 2016 (KR) 10-2016-0011747
Mar. 4, 2016 (KR) 10-2016-0026643

(51) Int. Cl. A61K 31/5377; A01K 67/027; A01K 67/0276; A61K 31/436; A61K 31/4375; A61K 31/501; A61K 31/519; A61K 31/711; A61P 25/08; C12N 15/85; C12N 15/90; C12Q 1/6883; G01N 33/68

(52) U.S. Cl. CPC A61K 31/5377; A01K 67/027; A01K 67/0276; A61K 31/436; A61K 31/4375; A61K 31/501; A61K 31/519; A61K 31/711; A61P 25/08; C12N 15/85; C12N 15/907; C12Q 1/6883; G01N 33/68; A01K 2217/075; A01K 2227/105; A01K 2267/0356; C12Q 2600/156

(57) ABSTRACT

Provided is a use of the prophylaxis, amelioration or therapy of intractable epilepsy, for example, Focal Cortical Dysplasia (FCD).

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: 77,464 bytes XML file named “Opp 20255,229US_SEQ.xml” created Oct. 27, 2025.

### TECHNICAL FIELD

The present invention relates to the prophylaxis, amelioration or therapy of intractable epilepsy, for example, Focal Cortical Dysplasia (FCD). Also, the present invention relates to a biomarker panel for diagnosing intractable epilepsy, especially pediatric intractable epilepsy, and a method for diagnosing intractable epilepsy using the same.

### BACKGROUND ART

Epilepsy is a group of chronic neurological diseases characterized by repetitive seizures induced by excessive electric activity in some neurons within a short time, with the consequent incurrence of neurobiological, mental, perceptual, and social changes.

Epilepsy that is insensitive to anti-epileptic drugs developed thus far is called intractable epilepsy, and accounts for about 20% of all epilepsy cases diagnosed each year. Malformations of cortical developments (MCD) such as Focal Cortical Dysplasia (FCD), Hemimegalencephaly (HME) and Tuberous Sclerosis Complex (TSC), Hippocampal Sclerosis (HS), and Sturge-Weber Syndrome (SWS) are known as common causes of intractable epilepsy.

Insensitive to available antiepileptic drugs, intractable epilepsy requires neurosurgical treatment to resect a brain lesion to control epilepsy. Hence, there is a need for a technique for molecular biological diagnosis specific for malformations of cortical development or Hippocampal Sclerosis, both causative of intractable epilepsy.

FCD is an important cause of epilepsy that is difficult to control with available antiepileptic drugs, and this condition accounts for as much as 50% of juvenile patients undergoing epilepsy surgery. FCD is a sporadic developmental malformation of the cerebral cortex and is characterized by the disorganized structure of the cerebral cortex or cytological abnormalities of the neurons in focally affected regions.

Although surgical resection of FCD renders approximately 60% of patients seizure free, a significant portion of Focal Cortical Dysplasia patients continues to have seizures. Due to the lack of understanding of the molecular genetic etiology, the development of novel and more effective FCD therapies remains elusive. A long-held but unproven hypothesis is that FCD is caused by somatic mutations occurring in affected brain regions during brain development. However, no such mutations have been identified.

FCD can be classified into several types based on histopathology. In particular, FCD type II (FCDII) shows a homogenous pathology, characterized by disrupted cortical lamination, and dysmorphic neurons or balloon cells (Epilepsia 52, 158-174 (2011)). FCDII is diagnosed in 29-39% of all FCD patients who undergo epilepsy surgery (Brain 129, 1907-1916 (2006)). Although an association between human papilloma virus and FCDII has been reported, the molecular genetic etiology of FCDII remains poorly understood. Interestingly, the brain MRIs of FCDII patients are occasionally reported to be normal; however, microscopic examinations of surgical specimens reveal scattered dysmorphic cells surrounded by an abundance of normally appearing cells. These radiological and histopathological findings suggest that only a small fraction of cells containing somatic mutations exist in surgically resected tissue, and that these mutations might not be efficiently detected through capillary Sanger sequencing or typical whole exome sequencing (WES) with an average read depth of 100-150×.

Against this background, the present inventors have identified FCD-specific somatic mutations in the brain tissues of FCD patients undergoing epilepsy surgery, using deep sequencing strategies such as deep whole exome sequencing, hybrid capture sequencing, and amplicon sequencing, established a transgenic animal with FCD, using the somatic mutations, and found that the administration of an mTOR inhibitor to the transgenic animal restrains FCD symptoms, leading to the present disclosure.

### DISCLOSURE

### Technical Problem

An object of the present disclosure is to provide a kit or method for preventing, ameliorating or treating intractable epilepsy caused by the brain somatic mutations in components of the PI3K-AKT-mTOR pathway, or by Focal Cortical Dysplasia (FCD), Hemimegalencephaly (HME), Hippocampal Sclerosis (HS) or Sturge-Weber Syndrome (SWS), using an mTOR inhibitor as an active ingredient.

Another object of the present disclosure is to provide the use of an mTOR in preventing, ameliorating, or treating intractable epilepsy or a disease causing intractable epilepsy. Here, the intractable epilepsy may be caused by FCD. In particular, FCD may be associated with cerebral somatic mutation.

A further object of the present disclosure is to provide a pharmaceutical or food composition for preventing, ameliorating or treating intractable epilepsy caused by brain somatic mutations in components of the PI3K-AKT-mTOR pathway or by Focal Cortical Dysplasia (FCD), Hemimegalencephaly (HME), Hippocampal Sclerosis (HS) or Sturge-Weber Syndrome (SWS), comprising an mTOR inhibitor as an active ingredient.

A still further object of the present disclosure is to provide a diagnostic kit for intractable epilepsy, comprising an agent capable of detecting a mutation present in a gene or protein involved in a PI3K-AKT-mTOR pathway.

Still another object of the present disclosure is to provide a method for diagnosing intractable epilepsy, comprising detecting a mutation present in a gene or protein involved in the PI3K-AKT-mTOR pathway in a sample from a subject, using the diagnostic kit.

Yet a further object of the present disclosure is to provide a mutant of a gene or protein involved in the PI3K-AKT-mTOR pathway.

Yet another object of the present disclosure is to provide a biomarker panel for the diagnosis of intractable epilepsy, comprising a mutant of a gene or protein involved in the PI3K-AKT-mTOR pathway.

Still yet another object of the present disclosure is to provide a composition for inducing intractable epilepsy, comprising a mutant of a gene or protein involved in the PI3K-AKT-mTOR pathway.

An additional object of the present disclosure is to provide a transgenic animal with intractable epilepsy, into which a gene or protein involved in the PI3K-AKT-mTOR pathway is introduced.

Another additional object of the present disclosure is to provide a method for inducing intractable epilepsy, comprising introducing a gene or protein involved in the PI3K-AKT-mTOR pathway into cells, ex vivo.

### Technical Solution

The present disclosure addresses use of an mTOR inhibitor in the prophylaxis, amelioration or therapy of intractable epilepsy or a causative disease of intractable epilepsy. Here, the intractable epilepsy may be caused by FCD. More particularly, the FCD may be FCD associated with brain somatic mutation.

Also, the present disclosure addresses a biomarker panel for the diagnosis of intractable epilepsy, and a diagnostic method of intractable epilepsy using the same. Within the scope of diseases causative of intractable epilepsy. Malformations of cortical developments (MCD) such as Focal Cortical Dysplasia (particularly, FCD type II), Hemimegalencephaly (HME) and Tuberous Sclerosis Complex (TSC), Hippocampal Sclerosis (HS), and Sturge-Weber Syndrome (SWS) fall

Hereinafter, a detailed description will be given of the present invention.

Intensive and thorough research and analysis of tissue samples resected from brains of patients with intractable epilepsy caused by Focal Cortical Dysplasia (FCD), Hemimegalencephaly (HME), Hippocampal Sclerosis (HS), or Sturge-Weber Syndrome (SWS) resulted in the finding that brain somatic mutations specific for components of the PI3K-AKT-mTOR pathway exist and that the mutations can be used as a biomarker panel for diagnosing intractable epilepsy. Also, the present inventors discovered the fact that cells into which the mutations are introduced overexpress mTOR, inducing intractable epilepsy, and succeeded in developing a method useful in preventing, ameliorating or treating Focal Cortical Dysplasia (FCD), Tuberous Sclerosis Complex (TSC), Hemimegalencephaly (HME), Hippocampal Sclerosis (HS) or Sturge-Weber Syndrome (SWS), and the intractable epilepsy caused by Focal Cortical Dysplasia (FCD), Tuberous Sclerosis Complex (TSC), Hemimegalencephaly (HME), Hippocampal Sclerosis (HS) or Sturge-Weber Syndrome (SWS).

In the present disclosure, brain tissue, saliva and blood samples were obtained from FCD-caused intractable epilepsy patients undergoing epilepsy surgery, and nucleotide sequencing revealed nine different mTOR gene mutations specifically present in patients with FCD-caused intractable epilepsy, nine mTOR protein mutations corresponding thereto, and six different genetic mutations in components of the PI3K-AKT-mTOR pathway and six corresponding protein mutations corresponding thereto (Table 1).

TABLE 1

| | | Mutation on | | |
|---|---|---|---|---|
| No. | Gene | mTOR Gene | mTOR Protein | Remark |
| 1 | mTOR | C616T | R206C | Cytosine (C) at position 616 → Thymine (T) |
| | | | | Arginine (R) at position 206→ Cysteine (C) |
| 2 | mTOR | G1871A | R624H | Guanine(G) at position 1871 → Adenine(A) |
| | | | | Arginine(R) at position 624 → Histidine (H) |
| 3 | mTOR | T4348G | Y1450D | Thymine (T) at position 4348 → Guanine(G) |
| | | | | Tyrosine (Y) at position 1450 → Aspartic acid (D) |
| 4 | mTOR | T4447C | C1483R | Thymine(T) at position 4447 → Cytosine(C) |
| | | | | Cysteine(C) at position 1483 → Arginine(R) |
| 5 | mTOR | G5126A | R1709H | Guanine(G) at position 5126 → Adenine(A) |
| | | | | Arginine(R) at position 1709→ Histidine(H) |
| 6 | mTOR | C5930A | T1977K | Cytosine(C) at position 5930→ Adenine(A) |
| | | | | Threonine (T) at position 1977 → Lysine (K) |
| 7 | mTOR | C6577T | R2193C | Cytosine(C) at position6577 → Thymine(T) |
| | | | | Arginine(R) at position 2193→ cysteine(C) |
| 8 | mTOR | C6644T | S2215F | Cytosine(C) at position 6644→ Thymine(T) |
| | | | | Serine (S) at position2215 → Phenylalanine (F) |
| 9 | mTOR | T7280C | L2427P | Thymine(T) at position 7280→Cytosine(C) |
| | | | | Leucine (L) at position 2427 → Proline (P) |
| 10 | mTOR | T7280A | L2427Q | Thymine(T) at position 7280→ Adenine(A) |
| | | | | Leucine (L) at position 2427→ Glutamine (Q) |
| 11 | TSC1 | C64T | R22W | Cytosine(C) at position 64→ Thymine(T) |
| | | | | Arginine(R) at position 22→ Tryptophan (W) |
| 12 | TSC1 | C610T | R204C | Cytosine(C) at position 610→ Thymine(T) |
| | | | | Arginine(R) at position 204→ cysteine(C) |
| 13 | TSC1 | G2432T | R811L | Guanine(G) at position 2432→ Thymine(T) |
| | | | | Arginine(R) at position 811→ Leucine (L) |
| 14 | TSC2 | G4639A | V1547I | Guanine(G) at position 4639 → Adenine(A) |
| | | | | Valine (V) at position 1547→ Isoleucine (I) |
| 15 | AKT3 | G740A | R247H | Guanine(G) at position 740 → Adenine(A) |
| | | | | Arginine(R) at position 247 → Histidine(H) |
| 16 | PIK3CA | G3052A | D1018N | Guanine(G) at position 3052→Adenine(A) |
| | | | | Aspartic acid (D) at position 1018→ Asparagine (N) |

The identified mutations were specifically detected in the brain tissue samples, but were negative for all available saliva and blood samples from mutation-positive patients. In addition, at least one of the nine different mTOR mutations was found in all FCD patients, with an allelic frequency of 1.26 to 12.6%.

In the present disclosure, mTOR mutant constructs expressing the mutations were obtained, and cells, after transfection of the mTOR mutant constructs thereinto, were analyzed for mTOR protein activation in terms of the phosphorylation of an S6 protein and the activity of mTOR kinase. As a result, an increase in the phosphorylation of S6 phosphorylation (FIG. 2a) and the activation of mTOR kinase (FIG. 2n) was identified, indicating that the hyperactivation of the mTOR pathway induces the phosphorylation of S6.

In addition, treatment with rapamycin, everolimus, or compounds of Chemical Formulas 1 to 4 was discovered to reduce the phosphorylation of S6 in the cells with the hyperactivated mTOR pathway due to the transfection of the mTOR mutant constructs thereinto (FIGS. 9a to 9c).

Meanwhile, the induction of FCD by the mTOR mutations provided by the present disclosure was confirmed by observing a significantly elevated level of phosphorylated S6 protein and an increased soma size of neurons in pathological brain samples of patients with FCD-caused intractable epilepsy (mTOR gene mutation) (FIGS. 2c to 2e), and by significant disruption of neuronal migration and a significantly increased level of phosphorylated S6 in the cortex of mTOR mutant construct-injected mice at embryonic day 14 (FIGS. 11b and 11c).

In the present disclosure, the lateral ventricles of embryonic mice were electroporated with mTOR mutant constructs carrying the genetic mutations at embryonic day 14 (E14), and video-electroencephalographic (video-EEG) monitoring of the mice was performed starting 3 weeks after birth. As a result, spontaneous seizure with epileptic discharge was detected in mice transfected with plasmids carrying the mTOR mutant genes of the present disclosure (FIGS. 12a and 12b). Further, it was observed that the soma sizes of GFP-positive neurons were greatly increased in affected cortical regions of electroporated mice carrying mTOR mutant constructs, showing an abnormal neuronal morphology similar to cytomegalic neurons. (FIG. 3d).

In addition, the animal models with spontaneous seizures or abnormal neurons, when administered with rapamycin, were observed to significantly decrease in the onset frequency of behavioral and electrographic seizures (FIG. 3c) and in the soma size of abnormal neurons (FIG. 3d).

In the present disclosure, it is revealed not only that the genetic or protein mutations are specifically detected in FCD patient samples, but also that the mutations can induce FCD. Further, an mTOR inhibitor, for example, rapamycin, everolimus, and compounds of Chemical Formulas 1 to 4, is found to suppress intractable epilepsy associated with the mTOR mutations, such as mTOR hyperactivation, spontaneous seizures, behavioral seizures, electrographic seizures and generation of abnormal neurons in FCD.

In the following Example section, mTOR mutant constructs carrying the somatic mutations were obtained and transfected into cells, with the consequence of an increased level of S6K phosphorylation, which explains the activation of mTOR. Rapamycin decreased the phosphorylation. The data suggest that given the mutations, mTOR, TSC1, TSC2, AKT3 and PIK3CA genes or proteins activate the mTOR pathway, thus inducing epilepsy.

According to another embodiment thereof, the present disclosure addresses a biomarker panel for the diagnosis of intractable epilepsy, comprising mTOR, TSC1, TSC2, AKT3 and PIK3CA genes or proteins carrying the mutations. Further, the present disclosure provides a diagnostic kit for detecting the biomarker panel genes or proteins in a sample from a subject, and a diagnostic method using the same. Moreover, the present disclosure provides a technique for constructing an epilepsy model, comprising inducing intractable epilepsy with the genetic or protein mutations.

Also, the present disclosure addresses the prophylaxis, amelioration or therapy of intractable epilepsy, and a composition, a kit or a method for preventing, ameliorating or treating malformations of cortical developments, Hippocampal Sclerosis, or Sturge-Weber Syndrome, such as FCD, Hemimegalencephaly, and Tuberous Sclerosis Complex, which are common causes of intractable epilepsy. A particular embodiment of the present disclosure relates to the prophylaxis, therapy and/or amelioration of brain somatic mutation-associated intractable epilepsy.

In detail, the intractable epilepsy of the present disclosure includes epilepsy caused by the brain somatic mutation of genes involved in the PI3K-AKT-mTOR pathway, and epilepsy caused by malformations of cortical developments, Hippocampal Sclerosis, or Sturge-Weber Syndrome, such as FCD, Hemimegalencephaly and Tuberous Sclerosis Complex.

As used herein, the term “epilepsy” refers to a group of chronic neurological diseases characterized by repetitive seizures induced by excessive electric discharge in some neurons within a short time. The term “intractable epilepsy” means epilepsy that is insensitive to available antiepileptic drugs. Forms of intractable epilepsy may be those that are caused by Malformations of Cortical Developments (MCD), Hippocampal Sclerosis (HS), or Sturge-Weber Syndrome (SWS), such as Focal Cortical Dysplasia (FCD), Hemimegalencephaly (HME), and Tuberous Sclerosis Complex (TSC).

In the normal development of the cerebral cortex, neurons migrate from one region of the brain to another to form a laminar structure. The term “Focal Cortical Dysplasia” or “FCD”, as used herein, is a congenital abnormality of brain development where the neurons in one area of the brain fail to migrate in the proper formation in utero and thus fail to form a normal laminar structure. Etiologically, FCD is accounted for by the failure of normal development in some region of the cerebrum or by the generation of some dysmorphic neurons even in a region that seems to develop normally, as observed in radiographic images. FCD occurs sporadically and is characterized by dysmorphic neurons and disrupted cortical lamination in affected cortical regions.

The brain somatic mutations associated with FCD may be mutations on mTOR genes or protein.

mTOR (mammalian target of rapamycin) protein, encoded by the FRAP1 gene in humans, is a serine/threonine protein kinase, which is functionally involved in cell growth, cell proliferation, cell death, cell survival, protein synthesis, and transcription, and belongs to the phosphatidylinositol 3-kinase-related kinase protein family. In the present disclosure, nucleotide and protein sequences of wild-type mTOR genes are represented by SEQ ID NOs. 1 and 2, respectively.

As used herein, the term “brain somatic mutation” means an alteration at one or more positions of the nucleotide sequence of a wild-type gene. In this context, it may be a nucleotide mutation of mTOR, TSC1, TSC2, AKT3 and PIK3CA genes or an amino acid mutation of proteins corresponding to the genes. By way of example, an alteration may occur on the nucleotide sequence of the wild-type mTOR gene represented by SEQ ID NO. 1. As shown in Table 1, the brain somatic mutation may be a substitution at one or more selected from the group consisting of positions 616, 1871, 4348, 4447, 5126, 5930, 6577, 6644, 7280 and 7280 of the nucleotide sequence of SEQ ID NO. 1.

Alternatively, the brain somatic mutation of the present disclosure may be an alteration at one or more positions of the amino acid sequence of the wild-type mTOR protein of SEQ ID NO. 2. On the amino acid sequence of SEQ ID NO. 2, for example, the mutation may include at least one selected from the group consisting of substitutions from arginine (R) at position 206 to cysteine (C), from R at position 624 to H, from Y at position 1450 to D, from C at position 1483 to R, from R at position 1709 to H, from T at position 1977 to K, from R at position 2193 to C, from S at position 2215 to F, from L at position 2427 to P, and from L at position 2427 to Q. The substituted amino acids may be encoded by the genetic codes resulting from mutations at corresponding positions on the nucleotide sequence of SEQ ID NO. 1. The base mutations and corresponding amino acid mutations are listed in Table 1.

As used herein, the term “TSC1 mutant gene” refers to a TSC1 gene in which a mutation occurs on the wild-type TSC1 nucleotide sequence of SEQ ID NO. 3. Particularly, it may be a gene containing at least one mutation selected from substitutions from cytosine (C) at position 64 to thymine (T), from cytosine (C) at position 610 to thymine (T), and from guanine (G) at position 2432 to thymine (T) on the nucleotide sequence of SEQ ID NO. 3.

As used herein, the term “TSC1 mutant protein” refers to a TSC1 protein in which a mutation occurs on the wild-type TSC1 amino acid sequence of SEQ ID NO. 4. Particularly, it may be a protein containing at least one mutation selected from substitutions from arginine (R) at position 22 to tryptophan (W), from arginine (R) at position 204 to cysteine (C), and from arginine (R) at position 811 to leucine (L) on the amino acid sequence of SEQ ID NO. 4.

The term “TSC2 mutant gene”, as used herein, refers to a gene in which a mutation occurs on the wild-type TSC2 nucleotide sequence of SEQ ID NO. 5. Particularly, it may be a gene containing a substitution from guanine (G) at position 4639 to adenine (A) on the nucleotide sequence of SEQ ID NO. 5.

The term “TSC2 mutant protein”, as used herein, refers to a TSC2 protein in which a mutation occurs on the wild-type TSC2 amino acid sequence of SEQ ID NO. 6. Particularly, it may be a protein containing a mutation from valine (V) at position 1547 to isoleucine (I) on the amino acid sequence of SEQ ID NO. 6.

The term “AKT3 mutant gene”, as used herein, refers to a gene in which a mutation occurs on the wild-type AKT3 nucleotide sequence of SEQ ID NO. 7. Particularly, it may be a gene containing a substitution from guanine (G) at position 740 to adenine (A) on the nucleotide sequence of SEQ ID NO. 7.

The term “AKT3 mutant protein”, as used herein, refers to a TSC2 protein in which a mutation occurs on the wild-type AKT3 amino acid sequence of SEQ ID NO. 8. Particularly, it may be a protein containing a mutation from arginine (R) at position 247 to histidine (H) on the amino acid sequence of SEQ ID NO. 8.

The term “PIK3CA mutant gene”, as used herein, refers to a gene in which a mutation occurs on the wild-type TSC2 nucleotide sequence of SEQ ID NO. 9. Particularly, it may be a gene containing a substitution from guanine (G) at position 3052 to adenine (A) on the nucleotide sequence of SEQ ID NO. 9.

The term “PIK3CA mutant protein”, as used herein, refers to a TSC2 protein in which a mutation occurs on the wild-type TSC2 amino acid sequence of SEQ ID NO. 10. Particularly, it may be a protein containing a mutation from aspartic acid (D) at position 1018 to asparagine (N) on the amino acid sequence of SEQ ID NO. 10.

In addition, the mutated proteins may contain additional mutations, so long as they do not entirely alter the activity of the molecules. Amino acid substitutions of proteins or peptides that preserve all of the activity of the molecules are known in the art (H. Neurath, R. L. Hill, The Proteins, Academic Press, New York, 1979). If necessary, the mTOR mutant proteins may be modified by, for example, phosphorylation, sulfation, acrylation, glycosylation, methylation, and/or farnesylation.

Examples of the mTOR inhibitor available in the present disclosure include those listed in the disclosures of the following Patent Application Nos. Danaferber cancer institute [symbol] PCT/US09/005656; Dolcetta, Diego [symbol] U.S. Ser. No. 14/400,469; Exelixis [symbol] PCT/US10/030354, U.S. Ser. No. 13/989,366, U.S. Ser. No. 12/784,254, U.S. Ser. No. 13/322,160, U.S. Ser. No. 13/988,948, U.S. Ser. No. 13/988,903, U.S. Ser. No. 13/989,156, U.S. Ser. No. 13/989,330, PCT/US12/042582, PCT/US10/035638, PCT/US10/035639; Sanofi [symbol] U.S. Ser. No. 13/381,571, U.S. Ser. No. 14/374,838; Infinity Pharmaceuticals [symbol] U.S. Ser. No. 12/199,689, U.S. Ser. No. 11/965,688, KR20097015914; Intellikine [symbol] U.S. Ser. No. 12/586,241, PCT/US09/005958, PCT/US09/005959, PCT/US09/049983, PCT/US09/049969, U.S. Ser. No. 14/238,426, U.S. Ser. No. 12/920,970, U.S. Ser. No. 12/920,966, U.S. Ser. No. 14/619,556; Takeda Pharmaceutical Company Limited [symbol] PCT/US10/000234, U.S. Ser. No. 12/841,940, U.S. Ser. No. 12/657,853, U.S. Ser. No. 12/657,854; S*Bio Pte Ltd [symbol] U.S. Ser. No. 13/001,099; Schering Corporation [symbol] PCT/US10/030350; The Reagents of The University of California [symbol] EP2012175019; Xuanzhu Pharma Corporation Limited [symbol] EP2013836950; KR20130049854; Signal RX Pharmaceuticals EP2009703974; Semafore Pharmaceuticals U.S. Ser. No. 11/962,612, U.S. Ser. No. 11/111,201, U.S. Ser. No. 10/818,145; Kudos Pharmaceuticals U.S. Ser. No. 13/014,275, U.S. Ser. No. 13/307,342, U.S. Ser. No. 11/842,927, U.S. Ser. No. 11/361,599, U.S. Ser. No. 11/817,134, PCT/GB06/000671; AstraZeneca [symbol] U.S. Ser. No. 11/667,064, U.S. Ser. No. 11/842,930, U.S. Ser. No. 11/844,092, U.S. Ser. No. 12/160,752, U.S. Ser. No. 12/170,128, U.S. Ser. No. 12/668,056, U.S. Ser. No. 12/668,059, U.S. Ser. No. 12/252,081, U.S. Ser. No. 12/301,722, U.S. Ser. No. 12/299,369, U.S. Ser. No. 12/299,359, U.S. Ser. No. 12/441,298, U.S. Ser. No. 12/441,305, U.S. Ser. No. 12/441,299, U.S. Ser. No. 12/441,301, U.S. Ser. No. 12/668,060, PCT/GB07/003414, PCT/GB07/003417, PCT/GB07/003454, PCT/GB07/003493, PCT/GB07/003497; Ariad Pharmaceuticals U.S. Ser. No. 10/862,149, U.S. Ser. No. 13/463,951, U.S. Ser. No. 14/266,291; Merck Sharp & Dohme Limited U.S. Ser. No. 13/263,193, U.S. Ser. No. 13/379,685, U.S. Ser. No. 13/520,274, U.S. Ser. No. 13/818,153, U.S. Ser. No. 13/818,177, U.S. Ser. No. 13/876,192, U.S. Ser. No. 14/234,837, PCT/US12/047522; Wyeth [symbol] U.S. Ser. No. 12/251,712, U.S. Ser. No. 12/354,027, U.S. Ser. No. 12/470,521, U.S. Ser. No. 13/950,584, U.S. Ser. No. 13/718,928, U.S. Ser. No. 14/477,650, U.S. Ser. No. 12/470,525, U.S. Ser. No. 12/050,445, U.S. Ser. No. 12/044,500, U.S. Ser. No. 12/473,605, U.S. Ser. No. 12/276,459, U.S. Ser. No. 12/363,013, U.S. Ser. No. 12/361,607, U.S. Ser. No. 12/397,590, U.S. Ser. No. 12/473,658, U.S. Ser. No. 12/506,291, U.S. Ser. No. 12/556,833, U.S. Ser. No. 12/558,661; Norvartis U.S. Ser. No. 12/599,131, U.S. Ser. No. 12/792,471, U.S. Ser. No. 12/792,187, U.S. Ser. No. 13/073,652; F.Hoffmann-La-Roche AG EP2012177885, U.S. Ser. No. 13/738,829, U.S. Ser. No. 12/890,810, U.S. Ser. No. 13/568,707, EP2010769036, PCT/EP10/067162; Genentech Inc U.S. Ser. No. 11/951,203, U.S. Ser. No. 12/821,998, U.S. Ser. No. 12/943,284.

In detail, mTOR inhibitors useful for the present disclosure may be as follows: AMG954, AZD8055, AZD2014, BEZ235, BGT226, rapamycin, everolimus, sirolimus, CC-115, CC-223, LY3023414, P7170, DS-7423, OSI-027, GSK2126458, PF-04691502, PF-05212384, temsirolimus, INK128, MLN0128, MLN1117, ridaforolimus, Metformin, XL765, SAR245409, SF1126, VS5584, GDC0980, and GSK2126458. Further examples of the mTOR inhibitors includes those listed in WO2012/104776, KR 10-1472607B, WO2010/039740, U.S. Pat. Nos. 8,846,670, 8,263,633, and WO2010/002954.

According to some embodiments of the present disclosure, the mTOR inhibitor may be at least one selected from the group consisting of rapamycin or a salt thereof, everolimus or a salt thereof, a compound of Chemical Formula 1 or a salt thereof, a compound of Chemical Formula 2 or a salt thereof, a compound of Chemical Formula 3 or a salt thereof, and a compound of Chemical Formula 4 or a salt thereof.

As used herein, the term “rapamycin” refers to a macrolide lactone compound, known as sirolimus, which has immunosuppressant functions. Rapamycin is commercialized as a drug for preventing rejection of transplanted organs. Also, it is used as a therapeutic agent for pneumonia, immunoinflammatory skin disorders such as systemic lupus erythematosus and psoriasis, immunoinflammatory bowel disorders, orbital inflammation, restenosis, and rheumatoid arthritis, and as an anti-cancer agent. However, nowhere has the application of rapamycin to the prevention or treatment of brain somatic mutation-associated FCD been reported in previous documents.

As used herein, the term “everolimus” refers to a drug for treating kidney cancer. It is used when the antiangiogenic drug SUNItinib or sorafenib is no longer effective. Also, everolimus is approved for the treatment of subependymal giant cell astrocytoma associated with Tuberous Sclerosis (TS) in patients who are not suitable candidates for surgical intervention. However, to date there have been no reports on the use of everolimus in the prevention or treatment of brain somatic mutation-associated FCD.

The compounds of Chemical Formulas 1 to 4 are known as inhibitors active against mTOR; however, applicability to the prevention or treatment of brain somatic mutation-associated FCD has not been known at all.

Rapamycin, everolimus, and the compounds of Chemical Formulas 1 to 4 are useful in the present disclosure, and their derivatives or mimics, pharmaceutically acceptable salts thereof, and hydrates are also fall within the scope of drugs available for the present disclosure

The pharmaceutically acceptable salts or hydrates may be salts or hydrates derived from inorganic acids or organic acids. Examples of pharmaceutically acceptable salts include salts of hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methane sulfonic acid, benzene sulfonic acid, and toluene sulfonic acid, but are not limited thereto. The hydrates may refer to those formed by binding rapamycin, everolimus or the compounds of Chemical Formulas 1 to 4 with water molecules.

As used herein, the term “treatment” or “therapy” refers to any action resulting in improvement or mitigation in the symptoms of a disease of interest, a reduction in affected area, the suppression or delay of the onset or progression of disease, the amelioration, mitigation, or stabilization of a disease state, partial or complete recovery, prolonged survival, and other beneficial alterations. In this regard, the term is intended to include the mitigation, amelioration, reduction or cure of symptoms of brain somatic mutation-associated FCD thanks to the administration of the mTOR inhibitor of the present disclosure into patients.

The symptoms of brain somatic mutation-associated FCD occur as neurons in an area of the brain fail to migrate in the proper formation during brain development, and are exemplified by spontaneous seizures, behavioral seizures, electrographic seizures, and the generation of abnormal neurons in the cerebral cortex.

Accordingly, the treatment according to the present disclosure may mean a significant decrease in the onset frequency of spontaneous seizures, behavioral seizures, and electrographic seizures and in the number or soma size of abnormal neurons in the cerebral cortex thanks to the administration of an mTOR inhibitor, for example, rapamycin, everolimus, and/or the compounds of Chemical Formulas 1 to 4, into patients with brain somatic mutation-associated FCD.

Depending on modalities and regimens of the pharmaceutical composition of the present disclosure, an effective amount of the mTOR inhibitor may be suitably determined by those skilled in the art.

For example, the pharmaceutical composition may comprise the mTOR inhibitor in an amount of 0.1 to 10% by weight based on the total weight of the composition, and particularly in an amount of 0.5 to 5% by weight.

The pharmaceutical composition of the present disclosure may comprise the mTOR inhibitor alone or in combination with a pharmaceutically acceptable additive. The pharmaceutically acceptable additive is an additive that is typically useful for formulations, examples of which include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. Examples of pharmaceutically acceptable excipients available in the present disclosure include a lubricant, a humectant, a sweetener, an aromatic, an emulsifier, a flavoring agent, a suspending agent, and a preservative, but are not limited thereto. That is, pharmaceutically acceptable additives available in the pharmaceutical composition of the present disclosure may be selected according to the purpose of use by those skilled in the art without difficulty, and their amounts may be determined within the range that does not hinder the purpose and effect of the present disclosure.

The therapeutically effective amount of the pharmaceutical composition of the present disclosure may vary depending on various factors including a patient's state, and weight, the severity of disease, the dosage form of drug, the route of administration, the time of administration, etc. For a preferable effect, the active ingredient of the present disclosure may be administered in an amount of from 1 to 1000 mg/kg a day, preferably in an amount of from 50 to 500 mg/kg, and more preferably in an amount of 150 to 300 mg/kg. The composition of the present disclosure may be administered in a single dose or may be divided into multiple doses. Accordingly, the dosage does not limit the present disclosure in any aspect.

The pharmaceutical composition of the present disclosure may be administered to mammals such as rats, mice, livestock and humans via various routes. All of the administration methods are known, and, for example, the dosage may be administered orally, rectally, intravenously, intramuscularly, subcutaneously, or intracerebroventricularly.

Another embodiment of the present disclosure pertains to a food composition for preventing or ameliorating brain somatic mutation-associated FCD, comprising at least one selected from the group consisting of the mTOR inhibitors, for example, rapamycin or a salt thereof, everolimus or a salt thereof, the compound of Chemical Formula 1 or a salt thereof, the compound of Chemical Formula 2 or a salt thereof, the compound of Chemical Formula 3 or a salt thereof, and the compound of Chemical Formula 4 or a salt thereof. The compounds of Chemical Formulas 1 to 4 are as defined above.

The food composition may be added to foods, without change or together with other food or food ingredients, and used properly according to typical modalities. The mTOR inhibitor useful in the present disclosure may be determined suitably according to the purpose of use (prevention, health or therapeutic treatment). For a general food composition, the active ingredient may be contained in an amount of 0.01 to 10 parts by weight, based on 100 parts by weight of the composition, and particularly in an amount of 0.05 to 1 parts by weight. However, the content of the mTOR inhibitor may be below the range in a food composition that is intended for long-term ingestion with the aim of improving or controlling health.

The food composition may be applied to a health food for the prevention or amelioration of brain somatic mutation-associated FCD. There are no particular limitations on the kind of health food to which the composition of the present invention can be added. Examples of such a health food include meats, sausages, breads, chocolates, candies, snacks, confectionery, pizzas, ramen noodles, other noodles, gums, dairy products such as ice cream, various soups, beverages, teas, drinks, alcoholic beverages, vitamin complexes, etc., and all kinds of commonly accepted health foods. In addition, the food composition of the present disclosure may further comprise a cytologically acceptable additive. Although not significantly important, the content of the sitologically acceptable additive may generally be determined within a range of 0.01 to 0.1 parts by weight, based on 100 parts by weight of the composition of the present disclosure.

Another embodiment of the present disclosure addresses a kit or composition for the diagnosis of intractable epilepsy or a causative disease thereof, comprising an agent capable of detecting a mutation in a gene or protein involved in the PI3K-AKT-mTOR pathway, or a method for the diagnosis of intractable epilepsy or a causative disease thereof, using the same.

Another embodiment of the present disclosure addresses a biomarker panel for the diagnosis of intractable epilepsy, comprising a mutant of a gene or protein involved in the PI3K-AKT-mTOR pathway. A further embodiment of the present disclosure addresses a composition for inducing intractable epilepsy, comprising a mutant of a gene or protein involved in the PI3K-AKT-mTOR pathway.

The term “diagnosis”, as used herein, refers to identifying the presence or characteristics of a pathological condition. For the purpose of the present disclosure, “diagnosis” may mean identifying the onset of intractable epilepsy, and furthermore, the progression or aggravation of the disease.

As used herein, the terms “diagnostic marker” “marker for diagnosis” and “diagnosis marker” are intended to indicate a substance that is found specifically in a sample of a patient with intractable epilepsy and the detection of which accounts for the onset of intractable epilepsy. For the purpose of the present disclosure, the diagnostic marker of the present disclosure may mean mutant genes or proteins of mTOR, TSC1, TSC2, AKT3 and PIK3CA, which are present specifically in the affected brain regions of intractable epilepsy patients.

As used herein, the term “biomarker panel” is intended to include one or more of the biomarkers disclosed in the present disclosure. The biomarker panel can be detected using a detection agent (or reagent) that can directly or indirectly associate with or bind to a biomarker protein or gene present in a sample.

The brain somatic mutant associated with intractable epilepsy in accordance with the present disclosure may be a mutant of a gene or protein involved in the PI3K-AKT-mTOR pathway. For example, mutants of mTOR, TSC1, TSC2, AKT3 and PIK3CA genes or proteins may be available. These mutant genes or proteins are as described above.

In a particular embodiment, the agent capable of detecting a substitution may be a primer, a probe or an antisense nucleic acid that is specific for a substitution region.

Another embodiment of the present disclosure addresses a method for providing information on the diagnosis of intractable epilepsy, comprising:
(a) treating a sample of a subject with the diagnostic kit;
(b) detecting in the sample a biomarker panel containing at least one selected from the group consisting of the substitutions listed in Table 1; and
(c) determining the onset of intractable epilepsy if the biomarker panel containing one or more of the substitutions is detected.

Also, contemplated in accordance with another aspect of the present disclosure is a diagnostic kit for intractable epilepsy, comprising an agent capable of detecting the amino acid substitutions listed in Table 1.

In a particular embodiment, the agent capable of detecting the substitutions may be an antibody or aptamer specific for a substitution region.

According to another aspect, the present disclosure relates to a method for providing information on the diagnosis of intractable epilepsy, comprising:
(a) treating a sample of a subject with the diagnostic kit;
(b) detecting in the sample a biomarker panel containing at least one selected from the group consisting of the mutations listed in Table 1; and
(c) determining the onset of intractable epilepsy if the biomarker panel containing one or more of the substitutions is detected.

In a particular embodiment, the sample may be a brain tissue sample from the subject.

Another aspect of the present disclosure addresses a biomarker panel for the diagnosis of intractable epilepsy, comprising the mutant protein or the mutant gene.

The term “agent capable of detecting a substitution”, as used in the context of the detection of substitutions on nucleotide sequences, means a substance useful for detecting a substitution (mutation) on the nucleotide sequences of mTOR, TSC1, TSC2, AKT3 or PIK3CA in the sample. For example, the agent may be a primer, probe, or an antisense oligonucleotide capable of binding specifically or complementarily to a nucleotide sequence carrying a substitution. Particularly, the primer, the probe or the antisense oligonucleotide may specifically bind to the stretch carrying a substitution, but not to a wild-type sequence.

The term “complementary” as used herein means a level of complementarity sufficient to selectively hybridize with the nucleotide sequence under certain particular hybridization or annealing conditions, and particularly under physiological conditions, and is intended to include both substantial complementarity and perfect complementarity, particularly perfect complementarity.

In some embodiments of the present disclosure, the agent for detecting a substitution region of a gene may be an antisense oligonucleotide. The term “antisense oligonucleotide”, as used herein, encompasses a nucleic acid-based molecule complementary to a target mutation region to form a duplex with the mutation region. It is applicable to the detection of the gene biomarker panel of the present disclosure.

In other embodiments of the present disclosure, the agent for detecting a substitution region of a gene may be a primer or a probe. Because nucleotide sequences of mTOR, TSC1, TSC2, AKT3 and PIK3CA mutant genes useful in the present disclosure are revealed, a primer or a probe for specifically amplifying a predetermined region of the gene can be designed on the basis of the nucleotide sequences.

As used herein, the term “primer” refers to a short nucleic acid strand, typically 7 to 50 bases long, having a free 3′ hydroxyl group, which forms a base pair with a complementary template so as to serve as a starting point for the production of a new template strand. Primers are typically synthesized, but may be naturally occurring nucleic acids. The sequence of a primer need not be completely consistent with that of a target template, and may be accepted if it is sufficiently complementary to hybridize with the template. DNA synthesis or replication requires a suitable buffer, proper temperatures, polymerizing enzymes (DNA polymerase or reverse transcriptase), and four kinds of nucleotide triphosphates, in addition to primers. In the present disclosure, sense and antisense primers specific for mTOR polynucleotide can be used for PCR amplification so that the PCR products can be used to diagnose epilepsy. The length of the sense and antisense primers may be suitably altered depending on the information known in the art. Particularly, the primers useful in the present disclosure may be those applicable to the amplification of a mutant region of the gene provided by the present disclosure.

In other embodiments of the present disclosure, the agent for detecting a substitution region of a gene may be a probe. The term “probe”, as used herein, is intended to refer to a fragment of a nucleotide sequence, such as RNA or DNA, ranging in length from ones to hundreds of bases, which can bind specifically to an mRNA of interest and which is tagged with a label for detecting the mRNA of interest. The probe useful in the present disclosure may be constructed in the form of oligonucleotide probes, single-stranded DNA probes, double-stranded DNA probes, or RNA probes. In an embodiment of the present disclosure, the diagnosis of epilepsy may be achieved by determining whether a probe complementary to the mTOR mutant polynucleotide of the present disclosure hybridizes with the nucleotide sequence of interest. The selection of suitable probes and hybridization conditions may be modified according to information known in the art.

The primers or probes useful in the present disclosure may be chemically synthesized using a phosphoramidite solid support method or other well-known techniques. Their nucleotide sequences may be modified using various means known in the art, so long as their fundamental properties remain unchanged. Illustrative, non-limiting examples of the modification include methylation, capping, substitution of natural nucleotides with one or more homologues, and alternation between nucleotides.

The term “agent for detecting a substitution”, as used in the context of the detection of a substitution on an amino acid sequence, refers to a substance useful for detecting a mutant region of a biomarker panel protein in a sample of a patient. Particularly, the agent may be an antibody or aptamer specific for a protein composed of an amino acid sequence carrying a mutation provided by the present disclosure. In some embodiments, the antibody may be monoclonal or polyclonal.

The term “antibody”, as used herein, refers to a specific protein molecule that indicates an antigenic region. With respect to the purposes of the present invention, the antibody binds specifically to a mutant region of the biomarker panel protein of the present disclosure. This antibody can be produced from a protein that is encoded by the mutant gene, typically cloned into an expression vector using a conventional method. Also, partial peptides being producible from the protein which is encoded by the mutant gene fall within the scope of the antibody. To function as an antibody, the partial peptide is required to contain at least 7 amino acid residues, preferably 9 or more amino acid residues, and more preferably 12 or more amino acid residues. No particular limitations are imposed on the form of the antibodies of the present disclosure. Among them are polyclonal antibodies, monoclonal antibodies and fragments thereof which contain a paratope, and all immunoglobulin antibodies. Further, special antibodies such as humanized antibodies are also within the scope of the present invention.

In addition, the antibodies of the present disclosure which are used to detect the marker diagnostic of intractable epilepsy include functional fragments of antibody molecules as well as complete forms having two full-length light chains and two full-length heavy chains. The functional fragments of antibody molecules refer to fragments retaining at least an antigen-binding function, and include F_{ab}, F_{(ab)}, F_{(ab′)2}, Fᵥ and the like.

The agent for detecting a biomarker panel gene or protein in accordance with the present disclosure may be embodied into a kit. The kit according to the present disclosure is capable of detecting a biomarker panel gene or a protein. The kit of the present invention may comprise a primer, a probe or an antisense oligonucleotide for detecting a biomarker panel gene, or an antibody or an aptamer for detecting a biomarker panel protein, and one or more compositions, agents or devices suitable for analysis.

For instance, the kit for detecting a biomarker panel gene in accordance with the present disclosure may be a kit for diagnosing intractable epilepsy comprising elements necessary for DNA chip function. The DNA chip kit may comprise a substrate to which an agent for detecting a biomarker panel gene is immobilized, and a reagent, an agent and an enzyme for constructing a fluorescence-labeled probe. In addition, the substrate may contain an agent for quantitatively detecting a control gene or a fragment thereof. In addition, the kit designed to detect a biomarker panel gene may be a kit comprising elements necessary for PCR. Such a PCR kit may comprise a pair of primers specific for each of the mTOR mutant genes, test tubes or other suitable containers, reaction buffers (various pH and magnesium concentrations), deoxynucleotides (dNTPs), enzymes such as Tag-polymerase, a DNase inhibitor, an RNase inhibitor, DEPC-water, sterile water, and so on. In addition, the kit may further comprise a pair of primers specific for a gene useful as a quantitative control. Particularly, the kit may be a multiple PCR kit for simultaneously amplifying and analyzing biomarker panel genes through multiple PCR.

Alternatively, the kit for detecting a biomarker panel protein may comprise antibodies and elements necessary for the immunological detection of the antibodies, including a support, a suitable buffer, a coloring enzyme- or fluorescent-labeled secondary antibody, and a coloring substrate. Examples of the support include a nitrocellulose membrane, a 96-well plate made of polyvinyl resin or polystyrene resin, and slide glass. Among the coloring enzymes are peroxidase and alkaline phosphatase. FITC or RITC may be used as a fluorescent. ABTS (2,2′-azino-bis-(3-ethylbenzothiazoline-6-sulfonic acid) or OPD (o-phenylenediamine), or TMB (tetramethyl benzidine) is suitable as the coloring substrate.

Genomic DNA or total protein available in the method for detecting a biomarker panel in a sample of a subject according to the present disclosure may be separated using a process known in the art.

The term “a sample of a subject”, as used herein, is intended to encompass specimens including tissues and cells in which the biomarker panel gene or protein can be detected. Particularly, the sample may be a brain tissue sample, but is not limited thereto.

According to some embodiments of the present disclosure, the method for detecting a biomarker panel gene in a sample of a subject comprises amplifying a nucleic acid from the sample and determining the base sequence of the amplified nucleic acid.

The amplification of the nucleic acid may be achieved by polymerase chain reaction (PCR), multiplex PCR, touchdown PCR, hot start PCR, nested PCR, booster PCR, real-time PCR, differential display PCR (DD-PCR), rapid amplification of cDNA ends (RACE) PCR, inverse PCR, vectorette PCR, thermal asymmetric interlaced PCR (TAIL-PCR), ligase chain reaction, repair chain reaction, transcription-mediated amplification, self-sustained sequence replication, or selective amplification of target polynucleotide sequences.

To determine the base sequence of the amplified nucleic acid, Sanger sequencing, Maxam-Gilbert sequencing, Shotgun sequencing, pyrosequencing, hybridization by microarray, allele-specific PCR, dynamic allele-specific hybridization (DASH), PCR extension assay, TaqMan technique, automated DNA sequencing, or next-generation DNA sequencing may be used. The next-generation DNA sequencing may be performed using a DNA analyzing system widely known in the art, for example, 454 GS FLX manufactured by Roche, Genome Analyzer manufactured by Illumina, SOLid Platform manufactured by Applied Biosystems, etc.

The detection of a biomarker panel protein in a sample of a patient may be performed by Western blotting, ELISA, radioimmunoassay, radioimmunodiffusion, ouchterlony immunodiffusion, rocket immunoelectrophoresis, immunohistostaining, immunoprecipitation assay, complement fixation assay, FACS, or protein chip assay using an antibody or aptamer specifically detecting the corresponding amino acid mutation. With the analysis methods, an antigen-antibody complex between the mTOR mutant protein and the antibody thereof can be identified, and intractable epilepsy can be diagnosed by examining the antigen-antibody complex between the mutant protein and the antibody thereof.

As used herein, the term “antigen-antibody complex” is intended to refer to a product formed by the binding of a mutant protein to an antibody specific thereto. The antigen-antibody complex thus formed may be quantitatively determined by measuring the signal intensity of a detection label.

The detection label may be selected from a group consisting of enzymes, fluorescents, ligands, luminescents, microparticles, redox molecules, and radioactive isotopes, but not strictly limited thereto. If an enzyme is used as the detection label, available enzymes may include β-glucuronidase, β-D-glycosidase, β-D-galactosidase, urease, peroxidase or alkaline phosphatase, acetylcholinesterase, glucose oxidase, hexokinase and GDPase, RNase, glucose oxidase and luciferase, phosphofructokinase, phosphoenolpyruvate carboxylase, aspartate aminotransferase, phosphoenolpyruvate decarboxylase, β-lactamase or the like, but are not limited thereto. Examples of the fluorescents may include fluorescein, isothiocyanate, rhodamine, phycoerythrin, phycocyanin, allophycocyanin, o-phthalaldehyde, fluorescamine or the like, but are not limited thereto. The ligand may be exemplified by biotin derivatives, but is not limited thereto. Among the luminescents may be acridinium ester, luciferin, and luciferase. Representative of the microparticles are colloidal gold and colored latex, without limitation thereto. The redox molecules may include ferrocene, ruthenium complex, biologen, quinone, Ti ion, Cs ion, diimide, 1,4-benzoquinone, hydroquinone, K₄W(CN)₈, [Os(bpy)₃]²⁺, [RU(bpy)₃]²⁺, [MO(CN)₈]⁴⁻ or the like, but are not limited thereto. The radioactive isotope may be exemplified by, but are not limited to, ³H, ¹⁴C, ³²P, ³⁵S, ³⁶Cl, 51Cr, ⁵⁷Co, ⁵⁸Co, ⁵⁹Fe, ⁹⁰Y, ¹²⁵I, ¹³¹I, ¹⁸⁶Re or the like.

In one exemplary embodiment, the measurement of an antigen-antibody complex between a biomarker panel protein and an antibody thereof may be achieved through an ELISA assay. In this regard, various kinds of ELISA assay may be employed, including direct ELISA using a labeled antibody that recognizes antigen attached to a solid support, indirect ELISA using a labeled antibody that recognizes a capture antibody from the complex of the antibody that recognizes an antigen attached to the solid support, direct sandwich ELISA using a labeled antibody that recognizes the antigen of an antigen-antibody complex attached to the solid support, and indirect sandwich ELISA, in which an antibody recognizing the antigen of an antigen-antibody complex attached to a solid support is detected by a labeled secondary antibody.

Most useful is a sandwich ELISA assay in which an antibody attached to a solid support is reacted with a sample and then captured by a labeled antibody recognizing the antigen of an antigen-antibody complex to perform enzymatic staining or by a labeled secondary antibody recognizing the antigen of an antigen-antibody complex to perform enzymatic staining. The onset of intractable epilepsy can be determined by identifying the formation of a complex between the biomarker panel and the antibody.

In another embodiment, Western blotting may be carried out using one or more antibodies active against the biomarker panel. For example, total proteins are isolated from a sample and separated according to size via electrophoresis. The electrophoresed proteins are then transferred onto a nitrocellulose membrane and reacted with the antibody. Quantitative analysis of the formed antigen-antibody complex with a labeled antibody makes it possible to determine the onset of intractable epilepsy, based on the expression level of the mutant protein encoded by the mutant gene. Such detection may be carried out by investigating an antigen-antibody complex between a mutant protein and an antibody thereof.

Also, available in accordance with still another embodiment of the present disclosure is a protein chip in which an array of antibodies against biomarker panel proteins is immobilized at predetermined loci and high density on a substrate. The protein chip assay may comprise isolating total proteins from a sample, hybridizing the isolated proteins with the protein chip to form an antigen-antibody complex, reading the information displayed on the protein chip to identify the presence of a protein of interest, and determining the onset of intractable epilepsy.

When an mTOR mutant gene or protein is detected using any of the detection methods, diagnosis can be made of the onset of intractable epilepsy caused by malformations of cortical developments.

According to another aspect thereof, the present disclosure addresses a technique of constructing an epilepsy model, comprising inducing intractable epilepsy with the genetic mutation and protein mutation.

A particular embodiment pertains to a composition for inducing intractable epilepsy, comprising at least one selected from among mTOR, TSC1, TSC2, AKT3, and PIK3CA mutant genes or proteins.

Also, contemplated in accordance with another aspect of the present disclosure is an intractable epilepsy-induced animal into which a mutant gene or protein of at least one selected from among mTOR, TSC1, TSC2, AKT3 and PIK3CA is introduced.

In accordance with another aspect thereof, the present disclosure pertains to a method for inducing intractable epilepsy, comprising introducing a mutant gene or protein of at least one selected from among mTOR, TSC1, TSC2, AKT3 and PIK3CA into a cell, ex vivo.

As used herein, the term “induction” means effecting a change from a normal state into a pathological state. For the purpose of the present disclosure, the term “induction” as used in conjunction with epilepsy refers to induction of the onset of intractable epilepsy from a healthy state.

In some embodiments, epilepsy-induced cells can be prepared by introducing a mutant gene or protein of at least one of mTOR, TSC1, TSC2, AKT3, and PIK3CA into cells. The cells include brain cells or embryos. The cells into which the mutant gene or protein is introduced may be developed into an intractable epilepsy-induced animal. When the mutant gene or protein is introduced, mTOR hyperactivation may occur, causing a failure in neuronal migration and a significant increase in S6K phosphorylation, with the consequent induction of epilepsy.

mTOR, TSC1, TSC2, AKT3, and PIK3CA mutant proteins with mutations on their wild-type amino acid sequences may be obtained from natural sources by extraction and purification using a method widely known in the art. Otherwise, the mutant proteins can be prepared by chemical synthesis (Merrifield, J. Amer. Chem. Soc. 85:2149-2156, 1963) or by recombinant DNA technology.

For chemical synthesis, a polypeptide synthetic method widely known in the art may be used. In recombinant DNA technology, a nucleic acid encoding a protein having a mutation on its amino acid sequence is inserted into a suitable expression vector, which is then transformed into host cells. They are cultured to express the mutant protein, followed by recovering the mutant protein, having a mutation on the amino acid sequence thereof, from the host cells. After being expressed in selected host cells, the mutant protein can be isolated and purified using a typical biochemical separation technique, for example, treatment with a protein precipitant (salting-out), centrifugation, sonication, ultrafiltration, dialysis, various chromatographic techniques such as molecular sieve chromatography (gel filtration), adsorption chromatography, ion-exchange chromatography, and affinity chromatography. Typically, the techniques are used in combination to achieve a protein of high purity.

Nucleotide sequences coding for mTOR, TSC1, TSC2, AKT3, and/or PIK3CA proteins with a mutation on their wild-type amino acid sequences can be isolated from natural sources or prepared by chemical synthesis. Nucleic acids having such nucleotide sequences may be single- or double-stranded, and may be DNA molecules (genome, cDNA) or RNA molecules. For the chemical synthesis of nucleic acids, synthetic methods widely known in the art, such as those described in the literature (Engels and Uhlmann, Agnew (?) Chem. Int. Ed. Engl. 37:73-127, 1988) may be used, as exemplified by triester, phosphite, phosphoramidite and H-phosphonate methods, PCR and other autoprimer methods, oligonucleotide synthesis on solid supports or the like.

In a particular embodiment of the present disclosure, the mutant protein or gene may be introduced into cells, embryos or animals with the aid of a recombinant vector.

The term “vector”, as used herein, refers to a means for introducing a nucleotide sequence encoding a protein of interest into a host cell. Plasmid vectors, cosmid vectors, and viral vectors fall within the scope of such a vector. A suitable expression vector may include a signal sequence or leader sequence for membrane targeting or secretion, in addition to regulatory elements such as a promoter, an operator, an initiation codon, a termination codon, a polyadenylation signal, and an enhancer, and may be constructed into various structures depending on the purpose. The initiation codon and the termination codon may be generally considered a portion of a nucleotide sequence encoding a protein of interest, necessarily functional in an individual to whom a genetic construct has been administered, and must be in a frame together with the coding sequence. The promoter may be generally constitutive or inducible. Further, the expression vector may include a selectable marker for selecting host cells containing the vector. For a replicable expression vector, a replication origin is necessary. A vector may replicate by itself, or may be incorporated into a host genomic DNA.

In a particular embodiment, a vector carrying a gene is designed so that the gene is irreversibly incorporated into the genome of a host cell and is stably expressed for a long time in the cell.

The mutant protein or gene of the present disclosure may be introduced into cells, particularly brain cells. In addition, it may be introduced into embryos, preferably embryos at the stage of brain formation and development.

No particular limitations are imposed on the method of introducing proteins or genes. For example, a vector may be inserted into cells, using a method such as transformation, transfection or transduction. The vector introduced into cells continuously expresses a gene inserted thereinto in the cells so as to produce a mutant protein having a mutation on its wild-type amino acid sequence.

As used herein, the term “epilepsy-induced animal” is intended to refer to a non-human animal that is transformed to exhibit increased intracellular mTOR protein activity compared to normal cells. The transformation may be induced by introducing a vector expressing at least one mutant protein of mTOR, TSC1, TSC2, AKT3 and/or PIK3CA into cells. The transgenic animal with intractable epilepsy can be effectively used as an intractable epilepsy animal model.

As used herein, the term “animal model” or “disease model” is intended to refer to an animal that suffers from a disease similar to a specific human disease and thus can be used as a model for the study of etiology and pathogenesis. For use as an animal model, an animal should support prediction of the same effect as in humans and should be easily and reproducibly constructed. In addition, the disease of the animal model should have the same etiology as in humans, or should progress in a pattern similar to that in humans. Accordingly, mammalian vertebrates, which are similar in body structure to humans, for example with respect to the internal organs, immune system, and body temperature, and which suffer from diseases such as hypertension, cancer, immunodeficiency, etc. are suitable as animal models. The animals are particularly mammals, such as horses, sheep, pigs, goats, camels, antelopes, dogs, rabbits, mice, rats, guinea pigs, and hamsters, and more particularly rodents such as mice, rats, guinea pigs, and hamsters. A mouse is most frequently used in studying human diseases because of its various advantages including small size, potent fertility, easy feeding management, high resistance to disease, hereditary uniformity, and variety of strains. Another advantage for acceptance for use as an animal model is that mice can be transformed to exhibit diseases or symptoms identical or similar to those in humans.

The animal model of the present disclosure is an epilepsy model constructed by gene manipulation to express at least one of mutant proteins of mTOR, TSC1, TSC2, AKT3 and/or PIK3CA. Exhibiting the ability to induce intractable epilepsy, the mutant protein or gene of the present disclosure can be introduced into cells or embryos to construct an intractable epilepsy model.

In the present disclosure, for example, at least one of mutant proteins or genes of mTOR, TSC1, TSC2, AKT3 and PIK3CA is introduced into an animal embryo which can then be developed into an animal in which intractable epilepsy is induced. The mutant protein or gene can be introduced into embryos with the aid of a vector carrying the gene. No particular limitations are imposed on the technique of introducing the vector into an embryo. Particularly, the vector may be introduced into an embryo at the stage of cerebral cortex formation.

The animal model with epilepsy according to the present disclosure can be effectively applied to the study of gene functions, molecular mechanisms of epilepsy, and novel anti-epileptic drugs.

Another aspect of the present disclosure addresses a composition, a kit and a method for preventing, ameliorating or treating intractable epilepsy or a disease causative of intractable epilepsy. The disease causative of intractable epilepsy includes malformations of cortical developments (MCD) such as FCD, Hemimegalencephaly and Tuberous Sclerosis Complex, Hippocampal Sclerosis (HS), and Sturge-Weber Syndrome (SWS).

### Advantageous Effects

As described above, the administration of an mTOR inhibitor, for example, rapamycin, everolimus, and/or the compounds of Chemical Formulas 1 to 4, into patients with brain somatic mutation-associated FCD can bring about a significant decrease in the frequency of onset of brain somatic mutation-associated intractable epilepsy or its causes, for example, spontaneous seizures, behavioral seizures, and electrographic seizures, and in the number or soma size of abnormal neurons in the cerebral cortex. In addition, the present disclosure provides a biomarker panel effective for the diagnosis of intractable epilepsy and a technique for the diagnosis of intractable epilepsy using the same. Moreover, intractable epilepsy can be induced in accordance with the present disclosure, and epilepsy animal models thus constructed make it possible to study genetic functions, the molecular mechanisms of epilepsy, and novel anti-epileptic drugs.

### DESCRIPTION OF DRAWINGS

FIG. 1a shows post-operative brain MRI images of patients carrying mTOR mutations (FCD4, FCD6), and H&E staining of pathological samples from the patients. The arrowheads (white) indicate resected brain regions and the arrows (black) indicate cytomegalic neurons (Scale bar=50 um).

FIG. 1b shows sites of mTOR-associated somatic mutations as found in FCD patients through deep sequencing.

FIG. 1c shows evolutionarily conserved amino acid residues responsible for the mTOR-associated somatic mutations on the mTOR sequences.

FIG. 2a shows the results of Western blot analysis of S6 phosphorylation in mTOR mutation-expressing HEK293T cells. “P-S6” stands for phosphorylated S6 proteins, “S6” for S6 proteins, and “Flag” for flag proteins. “20% serum” represents a positive control with mTOR activity after exposure to 20% serum for 1 hr.

FIG. 2b shows the results of an in-vitro kinase assay for mTOR proteins in HEK293 cells expressing the mTOR mutations of the present disclosure.

FIG. 2c shows the results of an immunohistochemical assay for S6 phosphorylation level and soma size in pathological samples from patients with FCD-caused intractable epilepsy.

FIG. 2d shows the mean numbers of phosphorylated S6 proteins in representative cortical regions of patients with FCD-caused intractable epilepsy (number of counted cells=197-1182 per case).

FIG. 2e shows the mean soma sizes of neurons in representative cortical regions of patients with FCD-caused intractable epilepsy.

FIG. 3a shows schematic views of a procedure for electroporating embryos in utero at embryonic day 14 (E14) with a plasmid carrying mTOR mutations, followed by screening only mice exhibiting fluorescence with a flashlight (Electron Microscopy Science, USA) after birth, monitoring the mice for seizures through video-electroencephalography (video-EEG), and examining the effect of rapamycin after the onset of seizures. The “in-utero electroporation (E14)” schematically shows the electroporation of a plasmid carrying mTOR mutations at embryonic day 14, the “GFP screening at birth (PO)” schematically shows monitoring only of fluorescence-exhibiting mice with a flashlight (Electron Microscopy Science, USA) after the electroporated embryos are delivered, and the “Video-EEG monitoring (>3 weeks)” schematically shows recording video-EEG with implanted electrodes after video monitoring of seizure onset from the time of weaning (>3 weeks).

FIG. 3b shows the presence of spontaneous seizures in mice carrying mTOR mutations of the present disclosure, based on video-EEG recording. The “No. of GFP+pups” represents the number of mice that expressed GFP as a result of the introduction of mTOR mutations thereinto, and the “No. of mice with seizure” represents the number of mice that underwent seizures as a result of the introduction of mTOR mutations thereinto.

FIG. 3c shows frequencies of spontaneous seizures in mice carrying mTOR mutations of the present disclosure before and after rapamycin treatment. *p<0.05 and **p<0.01 (n=7-17 for each group, one-way ANOVA with Bonferroni's post test).

FIG. 3d shows changes in the soma size of GFP-positive neurons in mice carrying mTOR mutations of the present disclosure before and after rapamycin treatment.

FIG. 4 is a schematic view of the experimental design of the present disclosure, showing deep sequencing analysis in tissues from FCD patients, and in-vitro and in-vivo functional analysis.

FIG. 5a shows algorithms used to exploit brain-specific mutations using both Virmid (Genome Biology, 14 (8), R90 (2013)) and MuTect software (Nature Biotechnology, 31, 213 (2013)) with regard to the deep sequencing results.

FIG. 5b shows sequencing counts of reference (Ref) and mutant (Mut) alleles, and percentages of mutated alleles from deep whole exome sequencing and amplicon sequencing of FCD patient samples.

FIG. 6 shows somatic mutations of FCD, found by deep whole exome sequencing, as colored bars in the collapsed mode of an Integrative Genomic Viewer (IGV).

FIG. 7 shows brain MRI images from FCD patients. Arrows highlight the affected cortical regions.

FIG. 8 shows domain organizations and 3-D structures of the mTOR kinase prepared using PyMol 1 (the PyMOL Molecular Graphics System, Schrodinger, LLC). “FAT” stands for FRAP, ATM, and TRRAP domains of mTOR, “FRB” for FKBP12-rapamycin binding domain, and “KD” for N and C lobes of the kinase domain. The activation and catalytic loops are indicated in red and blue, respectively. ATPrS and Mg²⁺ are shown as sticks and spheres, respectively. Identified mutation sites in FCD patients are labeled in red.

FIG. 9a shows the effects of rapamycin on HEK293K cells expressing mTOR mutations.

FIG. 9b shows the effects of rapamycin on HEK293K cells expressing mTOR mutations. “P-S6K” represents phosphorylated S6 proteins and “S6K” represents S6 proteins.

FIG. 9c shows the effects of the compounds of Chemical Formulas 1 to 4 and everolimus on HEK293K cells expressing mTOR mutations. “P-S6K” represents phosphorylated S6 proteins and “S6K” represents S6 proteins.

FIG. 10 shows the enrichment of the mTOR mutant allele of the present disclosure, as analyzed by Sanger sequencing after cytomegalic neurons with increased S6 phosphorylation, obtained from pathological tissues of patients with FCD-caused intractable epilepsy, are micro-dissected. NeuN-positive cytomegalic neurons with increased S6 phosphorylation are labeled with yellow dots. “LCM” represents cytomegalic cells microdissected through laser capture microdissection. For a control, bulk genomic DNA, extracted from brain samples without enrichment, was used. Scale bar, 100 μm.

FIG. 11a is a schematic view of the procedure for in-utero electroporation with a plasmid carrying mTOR mutations of the present disclosure at E14, followed by the analysis of brain coronal sections at E18.

FIG. 11b shows images of coronal sections of mouse brains electroporated with mTOR mutants of the present disclosure, representing the disruption of neuronal migrations and mTOR activity. “CP” stands for cortical plate, “IZ” for intermediate zone, “SVZ” for subventricular zone, “VZ” for ventricular zone, “Wild type” for the insertion of a wild-type mTOR plasmid, and “Relative intensity value” for the relative intensity of a GFP (green fluorescent protein) in each case.

FIG. 11c shows mTOR activity in the developing mouse neocortex after the introduction of the mTOR mutations of the present disclosure (Scale bars, 20 μm, Error bars, s.e.m.).

FIG. 12a shows video-EEG records of spontaneous seizures in mice expressing the mTOR mutations of the present disclosure. “LF” stands for left frontal, “RF” for right frontal, “LT” for left temporal, and “RT” for right temporal.

FIG. 12b shows interictal spikes and nonconvulsive electrographic seizures in mice expressing the mTOR mutations of the present disclosure.

FIG. 12c shows a change in the frequency of interictal spikes in mice expressing the mTOR mutations of the present disclosure before and after rapamycin treatment.

FIG. 12d shows a change in the frequency of nonconvulsive electrographic seizures in mice expressing the mTOR mutations of the present disclosure before and after rapamycin treatment.

FIG. 12e shows the time of seizure onset in mice carrying a wild-type mTOR gene or a mTOR mutant of the present disclosure. (n=8-20 for each group). Error bars, s.e.m.

FIGS. 13 and 14 show the results of the treatment of HEK293T cells, expressing the mTOR mutations of the disclosure, with various mTOR inhibitors. “P-S6K” stands for phosphorylated S6 kinase, and “S6K” for S6 kinase.

FIG. 15 shows the results of Western blot analysis of HEK293T cells carrying wild-type or mutant TSC-1. (−) and (+) indicate a control and rapamycin treatment (200 nM), respectively. “P-S6K” stands for phosphorylated S6 kinase and “S6K” for S6 kinase.

FIG. 16 shows the results of Western blot analysis of HEK293T cells carrying wild-type or mutant TSC-2. (−) and (+) indicate a control and rapamycin treatment (200 nM), respectively. “P-S6K” stands for phosphorylated S6 kinase and “S6K” for S6 kinase.

FIG. 17 shows the results of Western blot analysis of HEK293T cells carrying wild-type or mutant AKT3. (−) and (+) indicate a control and rapamycin treatment (200 nM), respectively. “P-S6K” stands for phosphorylated S6K kinase and “S6K” for S6K kinase.

FIG. 18 shows the association of p.Arg22Trp and p.Arg204Cys mutations of TSC-1 with mTOR hyperactivation, as analyzed in Example 9. Immunoprecipitation assay results are given to identify the mechanism of TSC1 mutant-induced mTOR hyperactivation. “Empty” indicates non-treated cells.

FIG. 19 shows the results of the GTP-agarose pull down assay according to Example 9. The activity level of the TSC complex was analyzed by measuring the level of GTP-bound Rheb, a substrate of the TSC complex.

FIG. 20 shows results of the treatment of mutant mTOR-expressing HEK293T cells with rapamycin. **p<0.01 and ***p<0.001 (comparison with wild-type, n=3-5 for each group, one-way ANOVA with Bonferroni's post test)

FIG. 21 shows the results of the treatment of mutant mTOR-expressing HEK293T cells with rapamycin. “P-S6K” stands for phosphorylated S6 kinase, and “S6K” for S6 kinase.

FIG. 22 shows the results of the treatment of mutant mTOR-expressing HEK293T cells with the compounds of Chemical Formulas 1 to 4, and everolimus. “P-S6” stands for phosphorylated S6, and “S6” for S6 protein.

FIGS. 23a and 23b show the results of Western blot analysis of wild-type or mutant mTOR-expressing HEK293T cells before and after treatment with 6 different drugs according to Example 10. (−) and (+) indicate a control and rapamycin treatment (200 nM), respectively. “P-S6K” stands for phosphorylated S6 kinase, and “S6K” for S6 kinase.

FIGS. 24a and 24b show the results of Western blot analysis of wild-type or mutant TSC1-expressing HEK293T cells before and after treatment with 6 different drugs. (−) and (+) indicate a control and rapamycin treatment (200 nM), respectively. “P-S6K” stands for phosphorylated S6 kinase, and “S6K” for S6 kinase.

FIGS. 25a and 25b show the results of Western blot analysis of wild-type or mutant TSC1-expressing HEK293T cells before and after treatment with 6 different drugs. (−) and (+) indicate a control and rapamycin treatment (200 nM), respectively. “P-S6K” stands for phosphorylated S6 kinase, and “S6K” for S6 kinase.

FIGS. 26a and 26c show pathological samples from all FCD patients identified as carrying TSC1 and TSC2 mutations. “Non-FCD” stands for samples with normal brains, “P-S6” for phosphorylated S6 proteins, “NeuN” for a neuronal marker, and “Merge” for merged images of P-S6 and NeuN.

FIGS. 26b and 26d show percentages of cells expressing phosphorylated S6 in 4-5 cortical regions.

FIGS. 26e and 26f show the soma sizes of neuronal marker (NeuN)-positive neurons. *p<0.05, ** P<0.001, *** P<0.0001 [relative to Non-FCD samples, one-way ANOVA with Bonferroni posttest]. Error bars, s.e.m. Scale bars, 50 um.

FIG. 27a shows the disruption of neuronal migration in TSC mouse models, resulting in malformations of cortical developments. “Control” indicates the absence of sgRNA, and red letters indicate percentages of cells expressing the plasmids. Scale bars, 250 um. FIG. 27b shows the distribution of electroporated cells in the cortex. *p<0.05, *** P<0.0001 [Two-way ANOVA with Bonferroni posttest]. Error bars, s.e.m.

FIG. 28 shows electrographic seizures measured in TSC mouse models with spontaneous seizures after administration with rapamycin. *p<0.05 and **p<0.01 (n=7-17 for each group, one-way ANOVA with Bonferroni's post test)

### MODE FOR INVENTION

A better understanding of the present invention may be obtained through the following examples which are set forth to illustrate, but are not to be construed as limiting the present invention.

### Example 1: Identification of Gene by Whole Exome Sequencing, and Confirmation

### Example 1-1: Identification of 3 Candidate mTOR Mutations from 4 Patients Through

### Whole Exome Sequencing

Deep whole exome sequencing (read depth 412-668x) was performed on brain tissue samples from four FCDII patients (designated FCD3, FCD4, FCD6, and FCD23, respectively). Selection was made of three candidate genetic mutations that were found simultaneously using the two algorithms Virmid and Mutect.

To obtain data of the whole exome sequencinge, libraries of sequences were prepared using the Agilent library preparation protocols (Agilent Human All Exon 50 Mb kit) according to the manufacturer's instructions. The libraries were subjected to sequencing on Hiseq2000 (Illumina). For more accurate analysis, sequencing was carried out with a read depth of about 500×, five-times higher than the general sequencing depth. The sequencing data was prepared into a file that can be analyzed using the Best Practices Pipeline suggested by Broad Institute (https://www.broadinstitute.org/gatk/).

### Example 1-2: Confirmation of 3 Gene Mutant Candidates by Site-Specific Amplicon Sequencing and Identification of One Genetic Mutation (L2427P)

Site-specific amplicon was performed for the candidate mutations-(read depth, 100-347, 499×). The samples were obtained from the same brain tissue block through biological replication, thereby minimizing any unexpected sequencing artifacts or erroneous calls that can mimic low-frequency somatic mutations. For the site-specific amplicon sequencing, the samples were determined to have a mutation when the percentage of mutated reads exceeded 1%.

### Site-Specific Amplicon Sequencing

Two pairs of primers carrying two target regions of mTOR target gene codon sites (amino acids Cys1483 and Leu2427) were designed (Table 2).

TABLE 2

| Target region | primer | | SEQ ID NO |
|---|---|---|---|
| Chr1:11174301~Chr1:11174513 | Forward | 5′-TAGGTTACAGGCCTGGATGG-3′ | 11 |
| | Reverse | 5′-CTTGGCCTCCCAAAATGTTA-3′ | 12 |
| Chr1:11217133~Chr1:11217344 | Forward | 5′-TCCAGGCTACCTGGTATGAGA-3′ | 13 |
| | reverse | 5′-GCCTTCCTTTCAAATCCAAA-3′ | 14 |

Each primer had a patient-specific index, and only a single index was assigned to the sample from one patient so as to indicate patient origins of the base sequences upon the analysis of genetic mutations. PCR was performed on the target regions using the primers to amplify the nucleotide sequences of the two target regions. Subsequently, a DNA library was constructed using a Truseq DNA kit (Illumina) and libraries of the target genes were sequenced again on a Miseq sequencer (Illumina) (median read depth 135,424×). Using Bowtie2 (http://bowtie-bio.sourceforge.net/bowtie2/index.shtml), the sequences were aligned to a reference genome to generate analyzable files (bam files).

### Example 1-3: Sequencing Analysis Results

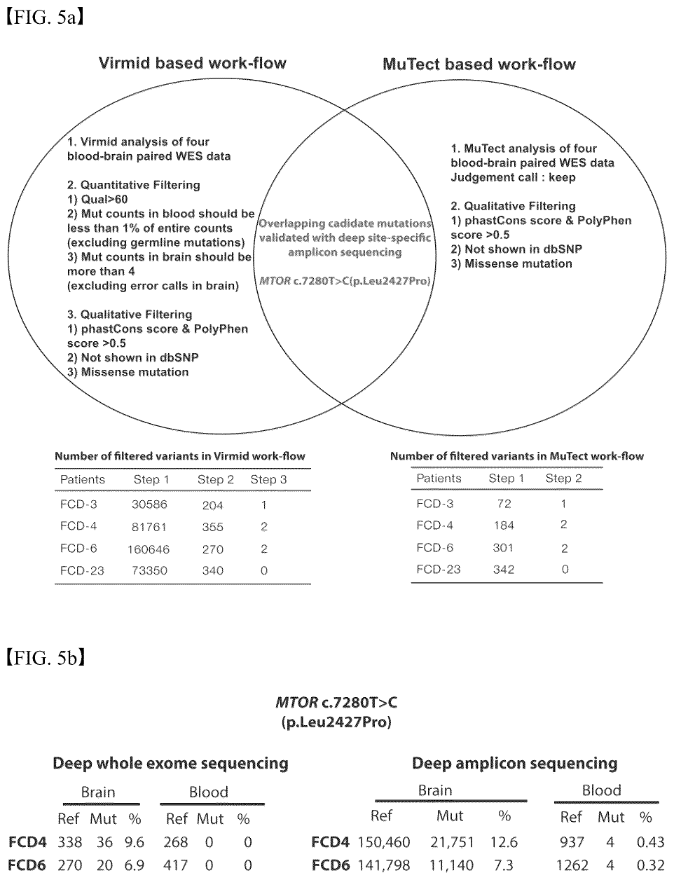

The use of two different sequencing platforms in biological replicates, as shown in FIG. 5a, revealed recurrent mTOR c.7280T>C encoding p.Leu2427Pro in two patients. The allelic frequencies in the affected brains, as shown in FIG. 5b, ranged from 9.6 to 12.6% in FCD4 and from 6.9 to 7.3% in FCD6.

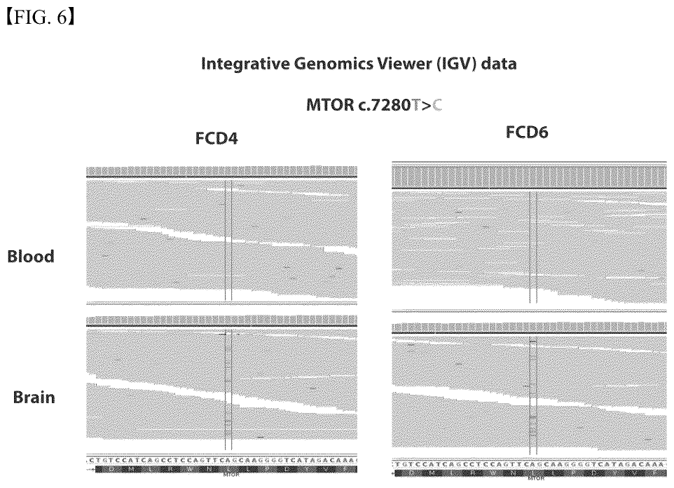

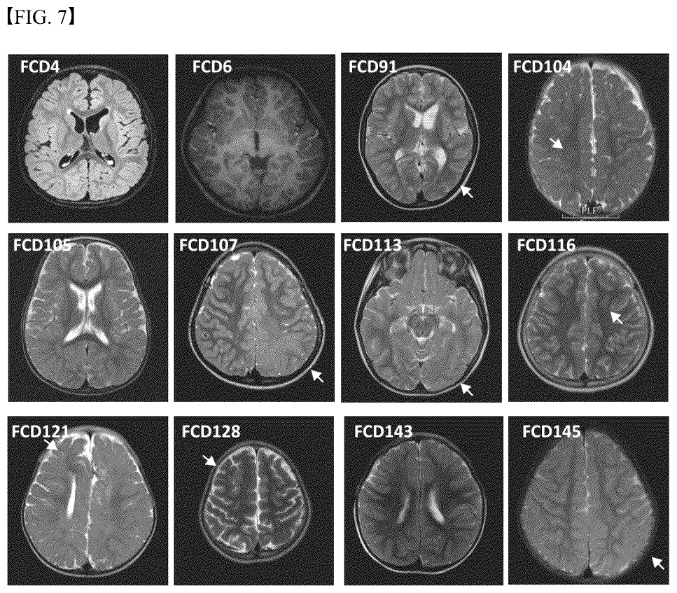

In addition, the mutation was negative in blood samples, as shown in FIGS. 5b and 6.

### Example 2: Search of mTOR-Specific Gene Mutation in Large Cohort

Based on the data of mTOR-specific genetic mutations obtained from 4 patients in Example 1, deep sequencing of the mTOR gene was performed for a large FCDII cohort including 73 patients.

### Example 2-1: Collection of Patient Samples and Extraction of Genomic DNA

From 73 focal cortical dysplasia (FCD)-caused intractable epilepsy patients undergoing epilepsy surgery, surgical brain tissues (1-2 g), saliva (1-2 ml), blood (about 5 ml), and formalin-fixed, paraffin-embedded brain samples were obtained with the consent thereof (Neurosurgery Dept. and Neurology Dept. of Severance Children's Hospital, Seoul, Korea). Genomic DNA was extracted from the freshly frozen brain, blood, saliva, and formalin-fixed, paraffin-embedded brain tissues using the following kits according to the instructions of the manufacturers:

Brain tissue: Qiamp mini DNA kit (Qiagen, USA), blood: Flexigene DNA kit (Qiagen, USA), saliva: prepIT2P purification kit (DNA Genotek, USA), formalin-fixed, paraffin-embedded brain tissue: Qiamp mini FFPE DNA kit (Qiagen, USA).

### Example 2-2: Sequencing

Hybrid capture sequencing of brain tissue samples from 73 FCDII patients were performed (read depth, 100-17,700×). PCR-based amplicon sequencing was carried out through site-specific amplicon sequencing (read depth, 100-347,499×, 73 patients) and mTOR amplicon sequencing (read depth, 100-20,210x, 59 patients).

For hybrid capture sequencing, an mTOR-specific probe was designed using SureDesign online tools (Agilent Technologies). Sequencing libraries were prepared using Agilent library preparation protocols according to the manufacturer's instructions. Sequencing was conducted using Hiseq2500 (Illumina) (median read depth 515x). The data obtained from the sequencing was prepared into analyzable files (bam files) using the Broad Institute best practice pipeline (https://www.broadinstitute.org/gatk/).

For mTOR amplicon sequencing, extracted genomic DNA was sequenced using customized MTOR amplicon (Truseq custom amplicon kit, Illumina) designed with illumine design studio (http://designstudio.illumina.com). DNA library preparation was performed according to the manufacturer's instructions. The libraries were sequenced on a Miseq sequencer (Illumina) (median read depth 1,647x). Analyzable bam files were generated using the BWA-MEM algorithm (http://bio-bwa.sourceforge.net).

### Example 2-3: Sequencing Result

In order to find brain-specific, de novosomatic mutations, blood-brain paired whole exome sequencing data sets were analyzed using both Virmid (http://sourceforge.net/projects/virmid/) and Mutect (http://www.broadinstitute.org/cancer/cga/mutect). Only the somatic mutations that were commonly found in the two analytic approaches were used in subsequent experiments.

Of the mutations commonly found in the hybrid capture sequencing and the PCR-based amplicon sequencing, only those meeting selection standards (depth 100 or greater, and 3 or more mutated calls (mapping quality 30 or higher) were selected as disease-related candidates.

For all 2508 CRAM files (compressed files) downloaded from the 1000 Genomes Project FTP, 9 somatic mutation positions were found (chr1: 11298590 for c.1871G>A, chr1: 11217330 for c.4348T>G, chr1: 11217231 for c.4447T>C, chr1: 11199365 for c.5126G>A, chr1: 11188164 for c.5930C>A, chr1: 11184640 for c.6577C>T, chr1: 11184573 for c.6644C>T, chr1: 11174395 for c.7280T>C and c.7280T>A). All of the 9 genomic positions were negative for the somatic mutations meeting the selection standards. Accordingly, the gene mutations identified in the present disclosure were found to be disease-specific.

### Example 2-4: Sequencing Result

Overlapping mutations in both hybrid capture sequencing (73 patients) and mTOR amplicon sequencing (59 patients) were detected in order to obtain a total of 9 true candidate variants (inclusive of mutations found in Example 1).

To rigorously exclude any potential sequence artifacts and erroneous calls, variants were considered as true only when identified variants (>1% mutated reads) were reproducible in both hybrid capture and amplicon sequencing as well as in multi-samples.

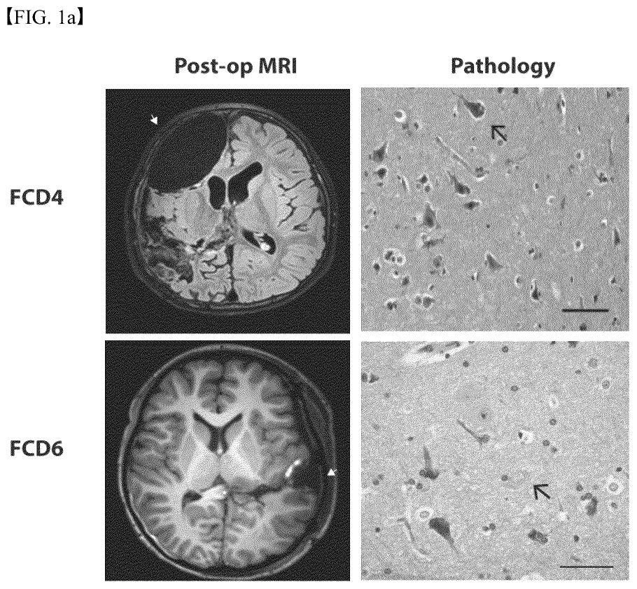

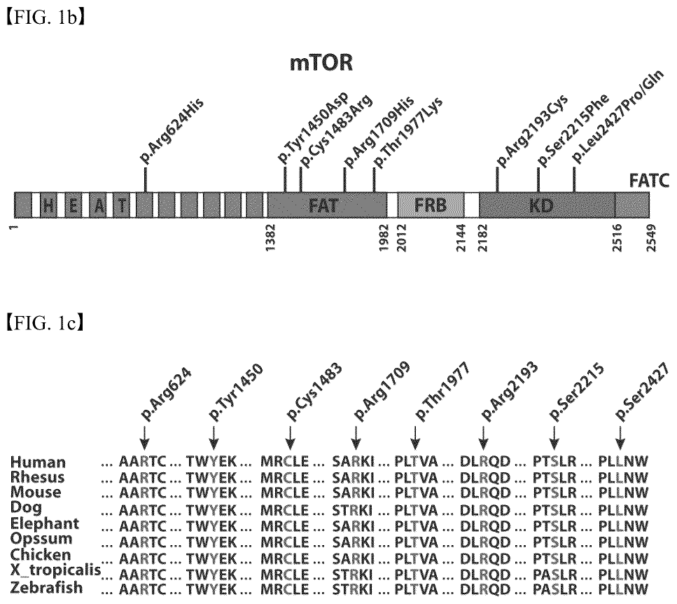

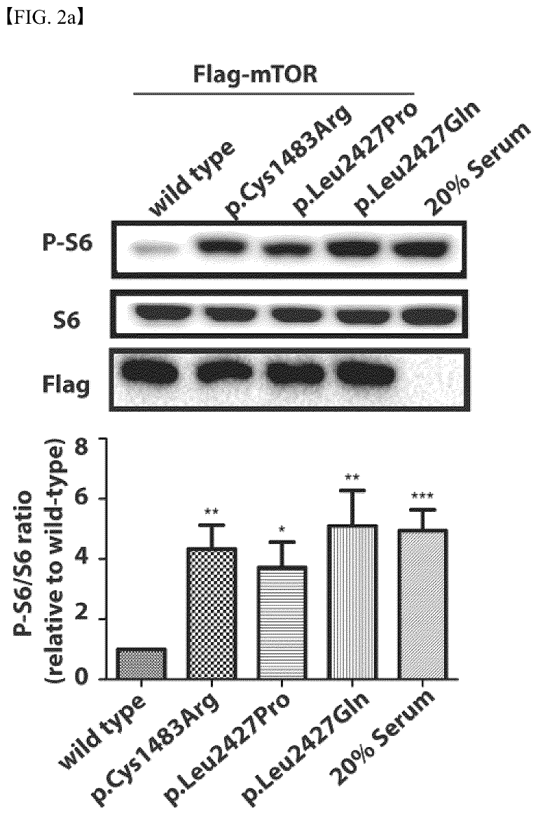

This analysis, as shown in FIG. 1b, revealed another 10 FCDII patients carrying 8 different somatic mutations in MTOR: mTOR c.1871G>A (p.Arg624His), c. 4348T>G (p.Tyr1450Asp), c.44471>C (p.Cys1483Arg), c.5126G>A (p.Arg1709His), c.5930C>A (p.Thr1977Lys), c.6577C>T (p.Arg2193Cys), c.6644C>T (p.Ser2215Phe), and c.7280T>A (p.Leu2427Gln). In total, 15.6% of the participants (12/77) were positive for 9 different brain somatic mutations in mTOR (Table 3).

TABLE 3

| | Age upon | | | mTOR gene | mTOR protein |
|---|---|---|---|---|---|
| Patient | Surgery | Sex | Pathology | mutation | mutation |
| FCD 4 | 5 years 2 months | F | Consistent with FCDIIa (Cortical | c.7280T > C | p.Leu2427Pro |
| | | | dyslamination/Dysmorphic neurons) | | |
| FCD 6 | 5 years | F | Consistent with FCDIIa (Cortical | c.7280T > C | p.Leu2427Pro |
| | | | dyslamination/Dysmorphic neurons) | | |
| FCD 91 | 7 years 1 month | F | Consistent with FCDIIa (Cortical | c.6577C > T | p.Arg2193Cys |
| | | | dyslamination/Dysmorphic neurons) | | |
| FCD 104 | 1 year 2 months | M | Consistent with FCDIIa (Cortical | c.1871G > A | p.Arg624His |
| | | | dyslamination/Dysmorphic neurons) | | |
| FCD 105 | 3 years 7 months | M | Consistent with FCDIIa (Cortical | c.5126G > A | p.Arg1709His |
| | | | dyslamination/Dysmorphic neurons) | | |
| FCD 107 | 7 years 3 months | F | Consistent with FCDIIb (Cortical | c.6644C > T | p.Ser2215Phe |
| | | | dyslamination/Dysmorphic neurons/ | | |
| | | | balloon cells) | | |
| FCD 113 | 10 years | F | Consistent with FCDIIb (Cortical | c.7280T > A | p.Leu2427Gln |
| | | | dyslamination/Dysmorphic neurons/ | | |
| | | | balloon cells) | | |
| FCD 116 | 7 years 9 months | M | Consistent with FCDIIb (Cortical | c.5930C > A | p.Thr1977Lys |
| | | | dyslamination/Dysmorphic neurons/ | | |
| | | | balloon cells) | | |
| FCD 121 | 11 months | M | Consistent with FCDIIb (Cortical | c.4348T > G | p.Tyr1450Asp |
| | | | dyslamination/Dysmorphic neurons/ | | |
| | | | balloon cells) | | |
| FCD 128 | 4 years 4 months | F | Consistent with FCDIIb (Cortical | c.4447T > C | p.Cys1483Arg |
| | | | dyslamination/Dysmorphic neurons/ | | |
| | | | balloon cells) | | |
| FCD 143 | 2 years 10 months | F | Consistent with FCDIIb (Cortical | c.6644C > T | p.Ser2215Phe |
| | | | dyslamination/Dysmorphic neurons/ | | |
| | | | balloon cells) | | |
| FCD 145 | 4 years 1 month | F | Consistent with FCDIIb (Cortical | c.5930C > A | p.Thr1977Lys |
| | | | dyslamination/Dysmorphic neurons/ | | |
| | | | balloon cells) | | |

All identified mutations were negative for all available saliva and blood samples from mutation-positive patients. 100% of the exomes from the 1000 Genomes database were mutation-negative. Among the mutations, p. Thr1977Lys, p.Ser2215Phe, and p.Leu2427Pro were recurrently found in two patients. All mutation-positive patients were found to have a single mTOR mutation. The allelic frequencies of identified mutations range from 1.26% to 12.6%. As can be seen in FIG. 1c, the affected amino acids are evolutionarily conserved.

### Example 3: Identification of mTOR Mutation-Induced mTOR Hyperactivation

To determine whether mTOR p.Tyr1450Asp, p.Cys1483Arg, p.Leu2427Gln, and p.Leu2427Pro lead to mTOR hyperactivation, HEK293T cells were transfected with wild-type or mutant mTOR vectors, and S6 phosphorylation, a well-known biomarker of mTOR activation along with S6K phosphorylation, was subjected to Western blot analysis.

### Example 3-1: Mutagenesis and mTOR Mutant Construct

A pcDNA3.1 flag-tagged wild-type mTOR construct was provided from Dr. Kun-Liang Guan at University of California, San Diego. This construct was used to generate mTOR mutant vectors (Y1450D, C1483R, L2427Q and L2427P) with a QuikChange II site-directed mutagenesis kit (200523, Stratagene, USA).

To construct pCIG-mTOR mutant-IRES-EGFP vectors, an annealing primer set [forward primer 5′-AATTCCAATTGCCCGGGCTTAAGATCGATACGCGTA-3′ (SEQ ID NO. 15) and reverse primer 5′-ccggtacgcgtatcgatcttaagcccgggcaattgg-3′ (SEQ ID NO. 16)] were inserted into pCIG2 (CAG promoter-MCS-IRES-EGFP) to generate pCIG-C1 having new restriction enzyme sites Mfel and MluI. Using the primers [hmTOR-Mfel-flag-F; gATCACAATTGTGGCCACCATGGACTACAAGGACGACGATGACAAGatgc (SEQ ID NO. 17) and hmTOR-MluI-R;tgatcaACGCGTttaccagaaagggcaccagccaatatagc (SEQ ID NO. 18)], PCR fragments corresponding to wild-type and mutated mTOR genes were subcloned into the Mfel and MluI sites of pCIG-C1 to construct pCIG-mTOR wild type-IRES-EGFP and pCIG-mTOR mutant-IRES-EGFP vectors, respectively. Primers for mutagenesis are listed in Table 4, below.

TABLE 4

| Name | Primer | | SEQ ID NO |
|---|---|---|---|
| Y1450D | Forward | 5′-TCGTGCAGTTTCTCATCCCAGGTAGCCTGGATC-3′ | 19 |
| | Reverse | 5′-GATCCAGGCTACCTGGGATGAGAAACTGCACGA-3′ | 20 |
| C1483R | Forward | 5′-GGCCTCGAGGCGGCGCATGCGGC-3′ | 21 |
| | Reverse | 5′-GCCGCATGCGCCGCCTCGAGGCC-3′ | 22 |
| L2427Q | Forward | 5′-GTCTATGACCCCITGCAGAACTGGAGGCTGATG-3′ | 23 |
| | Reverse | 5′-CATCAGCCTCCAGTTCTGCAAGGGGTCATAGAC-3′ | 24 |
| L2427P | Forward | GTCTATGACCCCTTGCCGAACTGGAGGCTGATG | 25 |
| | Reverse | CATCAGCCTCCAGTTCGGCAAGGGGTCATAGAC | 26 |

### Example 3-2. Transfection with Wild-Type and Mutant mTOR Vectors and Western Blot

HEK293T cells (Thermo Scientific) was cultured in DMEM (Dulbecco's Modified Eagle's Medium) containing 10% FBS at 37° C. and 5% CO₂. The cells were transfected with empty flag-tagged vector, flag-tagged mTOR wild type, and flag-tagged mTOR mutants using jetPRIME transfection reagent (Polyplus, France). The cells were serum-starved with DMEM containing 0.1% FBS for 24 hours after transfection and then incubated at 37° C. and 5% CO₂ in PBS containing 1 mM MgCl₂ and CaCl₂) for 1 hour. The cells were lysed with PBS containing 1% Triton X-100 and Halt protease and phosphatase inhibitor cocktail (78440, Thermo Scientific, USA).

Proteins were resolved by SDS-PAGE and transferred to PVDF membranes (Millipore, USA). The membranes were blocked with 3% BSA in TBS containing 0.1% Tween 20 (TBST). They were washed 4 times with TBST. The membranes were each incubated overnight at 4° C. with primary antibodies including a 1/1000 dilution of anti-phospho-S6 ribosomal protein (5364, Cell Signaling Technology, USA), anti-S6 ribosomal protein (2217, Cell Signaling Technology, USA), and anti-flag M2 (8164, Cell Signaling Technology, USA) in TBST. After incubation, the membranes were washed 4 times with TBST. They were incubated with a 1/5000 dilution of HRP-linked anti-rabbit or anti-mouse secondary antibodies (7074, Cell Signaling Technology, USA) for 2 hours at room temperature. After washing with TBST, immunodetection was performed using ECL reaction reagents.

### Example 3-3. Monitoring of S6 Phosphorylation Level in Mutant mTOR-Expression Cell

An in vitro mTOR kinase assay was performed. To this end, the kinase activity of mTOR was assayed using a K-LISA mTOR activity kit (CBA055, Calbiochem, USA) according to the manufacturer's protocol. The transfected cells (HEK293T cells) were lysed with TBS containing 1% Tween 20, Halt protease, and phosphatase inhibitor cocktail. One mg of total lysates was pre-cleared by adding 15 μl of protein G-beads (10004D, Life technologies, USA), and incubated at 4° C. for 15 min. Anti-flag antibodies were added to the pre-cleared lysates and incubated overnight at 4° C. Then, 50 μl of 20% slurry protein G-beads was added and incubated at 4° C. for 90 min. The supernatant was carefully discarded. The pelleted beads were washed 4 times with 500 μl of lysis buffer and once with the 1× kinase buffer of the K-LISA mTOR activity kit (K-LISA mTOR activity kit). The pelleted beads were resuspended in 50 μl of 2× kinase buffer and 50 μl of mTOR substrate (p70S6K-GST fusion protein), followed by incubation at 30° C. for 30 min. The reaction mixture was incubated in a glutathione-coated 96-well plate and incubated at 30° C. for 30 min. The phosphorylated substrate was detected using an anti-p70S6K-pT389 antibody, an HRP antibody-conjugate and a TMB substrate. Relative activities were measured by reading the absorbance at 450 nm.

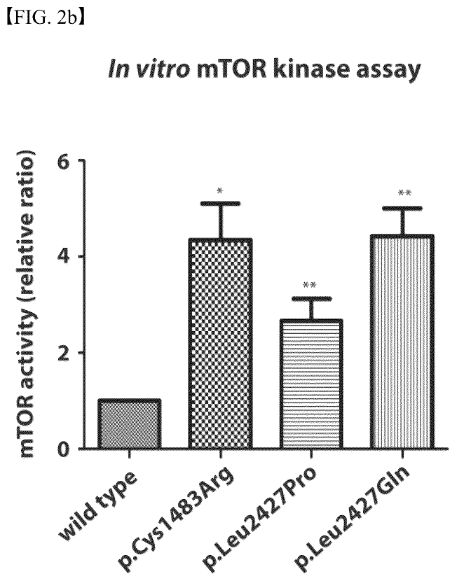

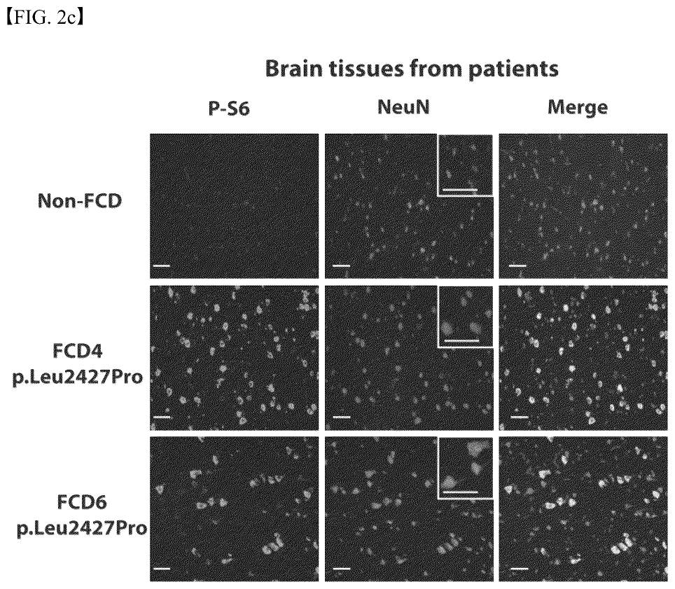

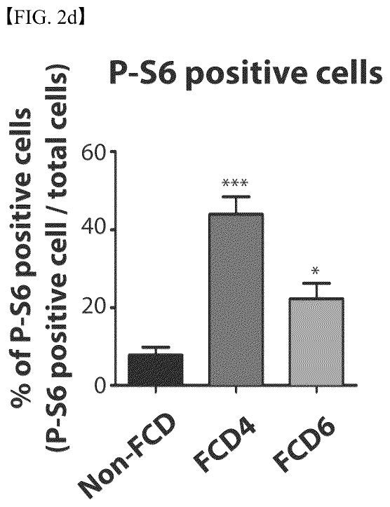

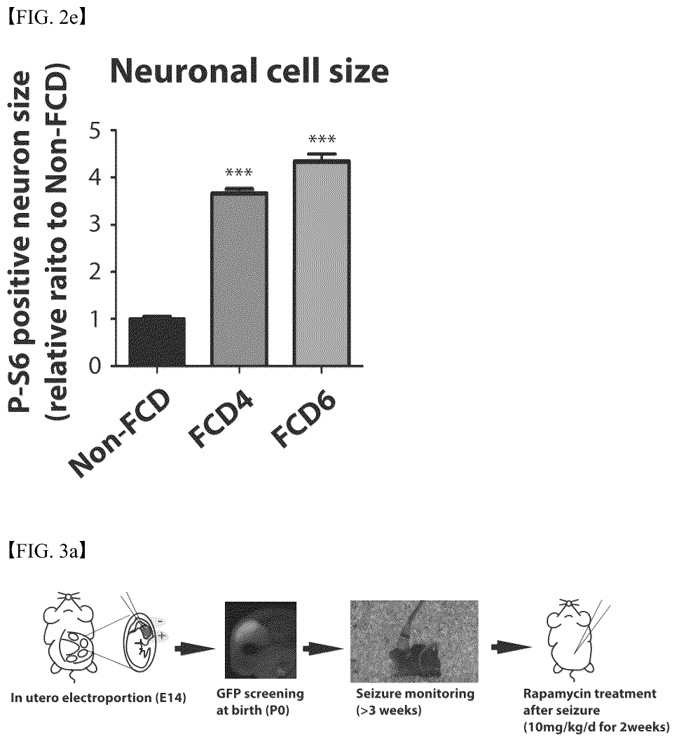

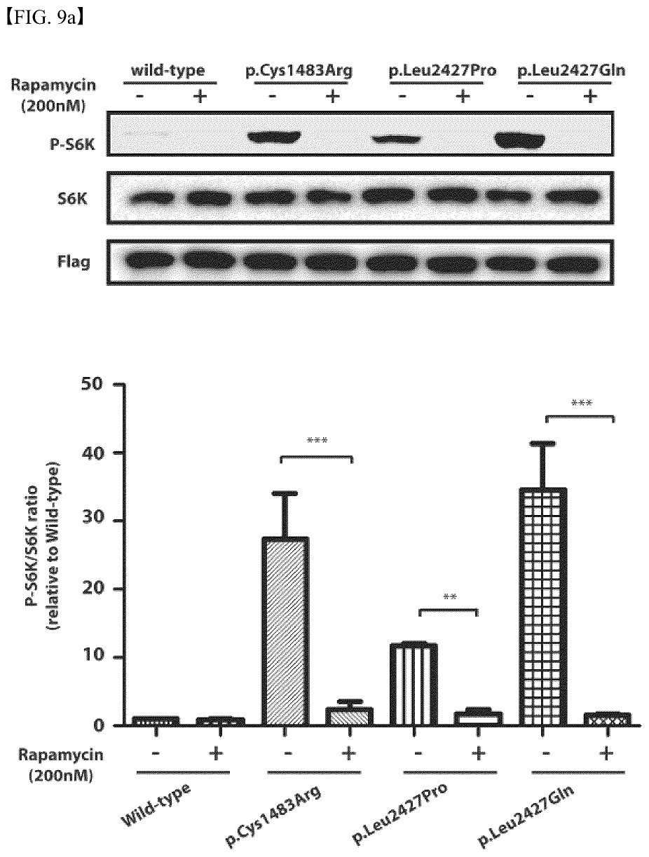

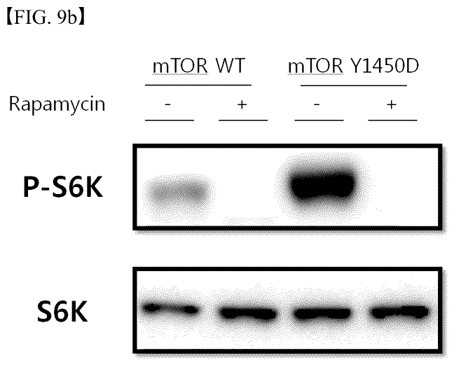

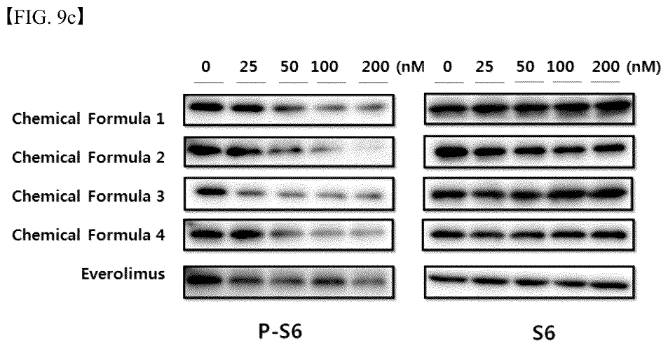

As is understood from the data of FIGS. 2a and 9b, S6 phosphorylation was robustly increased in mutant mTOR-expressing cells.

Wild-type and mutant mTOR proteins were pulled down from MTOR wild-type and mutant-expressing HEK293T cells, respectively, and assayed in vitro for mTOR kinase activity. As shown in FIG. 2b, pCys1483Arg, p.Leu2427Gln, and p.Leu2427Pro mTOR proteins had constitutively increased kinase activity.

### Example 3-4. Change in S6K Phosphorylation after Drug Treatment

After treatment with drugs (rapamycin, everolimus, compounds of Chemical Formulas 1 to 4), the mutant mTOR-expressing cells were analyzed for S6K phosphorylation.

Transfection of mTOR mutants into HEK293T cells was carried out in the same manner as in Example 3-2. Then, the cells were serum-starved with DMEM containing 0.1% FBS for 24 hours and incubated at 37° C. and 5% CO₂ in PBS containing 1 mM MgCl₂ and CaCl₂) for 1 hour, followed by treatment with rapamycin, everolimus, or compounds of Chemical Formulas 1 to 4 (Torin1, INK128, AZD8055, GSK2126458); Torin was purchased from TOCRIS, INK128, AZD8055, and GSK2126458 were provided from Selleckchem, and everolimus was purchased from LC laboratory. Subsequently, Western blotting was performed in the same manner as in Example 9.

As can be seen in FIGS. 9a and 9b, the phosphorylation of S6K in mutant mTOR-expressing cells was inhibited by rapamycin treatment.

In addition, the mutant mTOR-expressing cells were monitored for S6K phosphorylation after treatment with everolimus or the compounds of Chemical Formulas 1 to 4.

Likewise, everolimus or the compounds of Chemical Formulas 1 to 4 inhibited the phosphorylation of S6K in the mutant mTOR-expressing cells, as shown in FIG. 9c.

### Example 3-5. Change of S6K Phosphorylation with Various mTOR Inhibitors

Cells expressing various mTOR mutations were treated in the same manner as in Example 3-2 with rapamycin, everolimus, or the compounds of Chemical Formulas 1 to 4 and monitored for S6K phosphorylation. The mTOR mutations were R624H, Y1450D, C1483R, R1709H, Y1977K, S2215F, L2427P, and L2427Q.

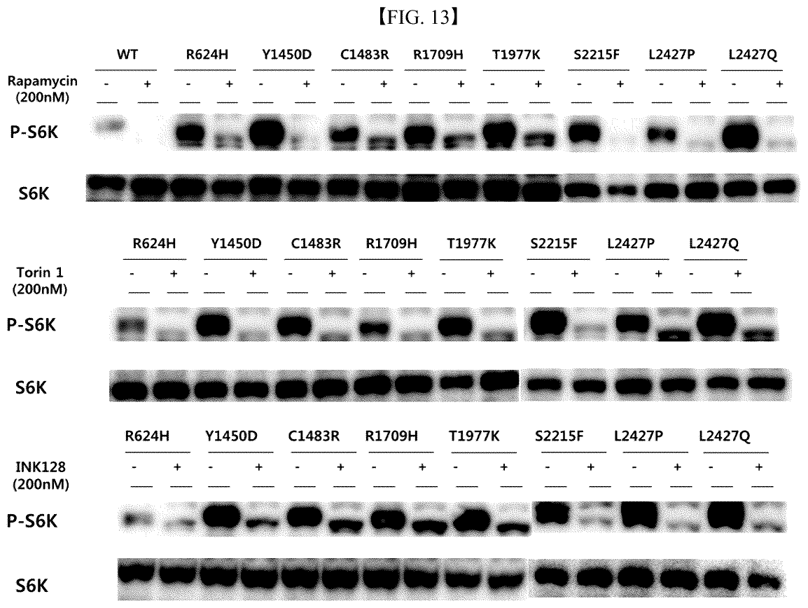

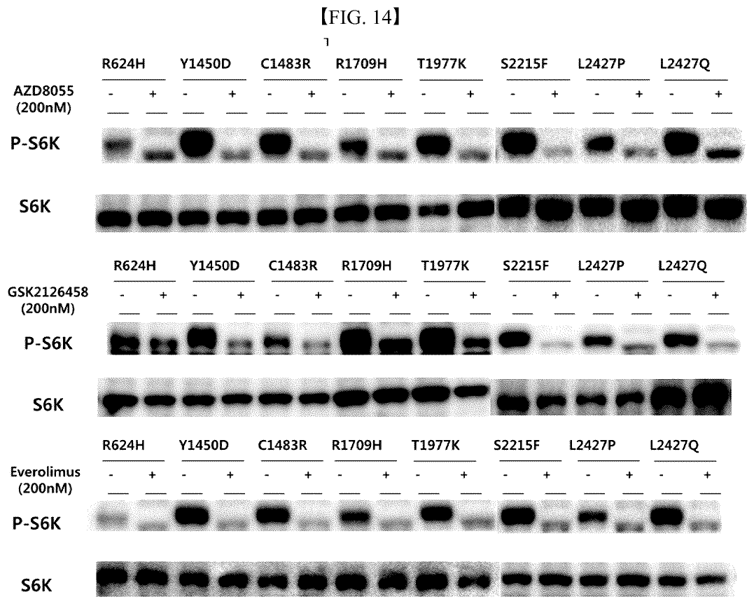

Briefly, the mutant mTOR-expressing cells were monitored for S6K phosphorylation level after treatment with everolimus or the compounds of Chemical Formulas 1 to 4. The results are depicted in FIGS. 13 and 14. As shown, the phosphorylation of S6K in all the mutant mTOR-expressing cells was inhibited by everolimus or the compounds of Chemical Formulas 1 to 4.

### Example 4: MTOR Hyperactivation Induced by mTOR Mutation

### Example 4-1: Immunostaining of Brain Tissue Section of FCD Patient

To determine whether the affected brains of FCDII patients carrying mutations are associated with mTOR hyperactivation, immunostaining was performed for S6 phosphorylation and NeuN (a neuronal marker) in brain tissue sections obtained from FCDII patients carrying the p.Leu2427Pro mutation.
Brain specimens that did not exhibit any malformations in cortical development (non-MCD) were collected in the operating room from the tumor-free margin of individual patients with glioblastoma as part of a planned resection, and were pathologically conformed to be normal brains without tumors. Surgical tissue blocks were fixed overnight in freshly prepared phosphate-buffered (PB) 4% paraformaldehyde, cryoprotected overnight in 20% buffered sucrose, and prepared into gelatin-embedded tissues blocks (7.5% gelatin in 10% sucrose/PB) before storage at −80° C. Cryostat-cut sections (10 um thick) were collected, placed on glass slides, and blocked in PBS-GT (0.2% gelatin and 0.2% Triton X-100 in PBS) at room temperature for 1 hr before staining with the following antibodies: rabbit antibody to phosphorylated S6 ribosomal protein (Ser240/Ser244) (1:100 dilution; 5364, Cell signaling Technology), and mouse antibody to NeuN (1:100 dilution; MAB377, Millipore). Subsequently, samples were washed in PBS and stained with the following secondary antibodies: Alexa Fluor 555-conjugated goat antibody to mouse (1:200 dilution; A21422, Invitrogen) and Alexa Fluor 488-conjugated goat antibody to rabbit (1:200 dilution; A11008, Invitrogen). DAPI included in a mounting solution (P36931, Life technology) was used for nuclear staining. Images were acquired using a Leica DMI3000 B inverted microscope. Cells positive for NeuN were counted using a 10× objective lens; 4-5 fields were acquired per subject within neuron-rich regions, and 100 or more cells were scored per region. The number of DAPI-positive cells represents total cell counts. Neuronal cell sizes were measured in NeuN-positive cells using the automated counting protocol of ImageJ software (http://rsbweb.nih.gov/ij/).

As seen in FIG. 2c, the results showed a marked increase in the number of neuronal cells positive for phosphorylated S6 in FCDII patients (FCD4 and FCD6) carrying p.Leu2427Pro mutation, but not in non-FCD brains, as shown in FIG. 2d. In addition, the cell sizes of phosphorylated S6-positive neurons were measured, and a robust increase in the soma size in pathological samples was observed, as shown in FIG. 2e.

### Example 4-2: Microdissection of S6 Phosphorylation-Increased, Cytomegalic Neurons in Brain Tissue of FCD Patient and Subsequent Sanger Sequencing

Surgical tissue blocks were fixed overnight in freshly prepared phosphate-buffered (PB) 4% paraformaldehyde, cryoprotected overnight in 20% buffered sucrose, and prepared into gelatin-embedded tissue blocks (7.5% gelatin in 10% sucrose/PB) before storage at −80° C. Cryostat-cut sections (10 um thick) were collected, placed on glass slides, and blocked in PBS-GT (0.2% gelatin and 0.2% Triton X-100 in PBS) at room temperature for 1 hr before staining with the following antibodies: rabbit antibody to phosphorylated S6 ribosomal protein (Ser240/Ser244) (1:100 dilution; 5364, Cell signaling Technology) and mouse antibody to NeuN (1:100 dilution; MAB377, Millipore). Subsequently, samples were washed in PBS and stained with the following secondary antibodies: Alexa Fluor 555-conjugated goat antibody to mouse (1:200 dilution; A21422, Invitrogen) and Alexa Fluor 488-conjugated goat antibody to rabbit (1:200 dilution; A11008, Invitrogen).

DAPI, included in a mounting solution (P36931, Life technology), was used for nuclear staining. After immunofluorescence staining, phosphorylated S6 immunoreactive neurons (n=about 20 per case) were microdissected with a PALM Laser capture system (Carl Zeiss, Germany) and collected in an adhesive cap (Carl Zeiss, Germany).

Thereafter, genomic DNA was extracted from the collected neurons using a QiAamp microkit (Qiagen, USA), and mutation regions (mTOR c.7280T>C) were amplified by PCR using the primers: sense 5′-CCCAGGCACTTGATGATACTC-3′ (SEQ ID NO. 27) and antisense, 5′-CTTGCTTTGGGTGGAGAGTT-3′ (SEQ ID NO. 28).

The PCR products thus obtained were purified with a MEGAquick spin total fragment purification kit (Intron, Korea), followed by Sanger sequencing with the aid of the BioDye Terminator and an automatic sequencer system (Applied Biosystems).

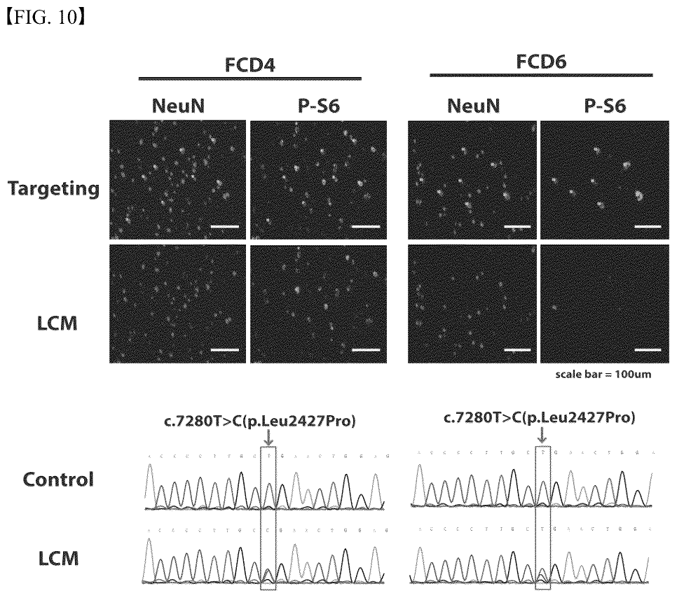

As shown in FIG. 10, moreover, the microdissection of cytomegalic neurons positive for phosphorylated S6 in the same pathological tissues showed the enrichment of the p.Leu2427Pro mutant allele in Sanger sequencing. These results suggest that the identified mTOR mutations are strongly associated with both the aberrant mTOR activation and the dysregulation of neuronal growth.

### Example 5: Effect of mTOR Hyperactivation on Cerebral Development in Animal Model

The recurrent mutation p.Leu2427Pro was selected for in vivo functional analysis. in utero electroporation of mTOR mutant constructs was performed to analyze the effect of the mTOR mutations on cortical radial neuronal migration and S6 phosphorylation in mice.

### Example 5-1: Construction of Animal Model

Timed pregnant mice (E14) (Damul Science) were anesthetized with isoflurane (0.4 L/min of oxygen and isoflurane vaporizer gauge 3 during surgery).

The uterine horns were exposed, and a lateral ventricle of each embryo was injected using pulled glass capillaries with 2 μg/ml of Fast Green (F7252, Sigma, USA) combined with 2-3 μg of mTOR mutant plasmids, constructed in Example 3-1, carrying mTOR C1483Y, mTOR E2419K, and mTOR L2427P mutants. Plasmids were electroporated on the head of the embryo by discharging 50 V with the ECM830 eletroporator (BTX-Harvard apparatus) in five electric pulses of 100 ms at 900-ms intervals.

### Example 5-2: Image Analysis of Mouse Model

Embryonic mice were electroporated at embryonic day 14 (E14). Then, their brains were harvested after 4 days of development (E18) and fixed overnight in freshly prepared phosphate-buffered (PB) 4% paraformaldehyde, cryoprotected overnight in 30% buffered sucrose, and prepared into gelatin-embedded tissue blocks (7.5% gelatin in 10% sucrose/PB) before storage at −80° C.

Cryostat-cut sections (30 um thick) were collected and placed on glass slides. DAPI, included in a mounting solution (P36931, Life Technology) was used for nuclear staining. Images were acquired using a Leica DMI3000 B inverted microscope or a Zeiss LSM510 confocal microscope. Fluorescence intensities reflecting the distribution of electroporated cells within the cortex were converted into gray values and measured from the ventricular zone (VZ) to the cortical plate (CP, using ImageJ software (http://rsbweb.nih.gov/ij/). Mander's co-localization analysis was carried out using Fiji software (http://fiji.sc/wiki/index.php/Colocalization_Analysis).

### Example 5-3: Experiment Result

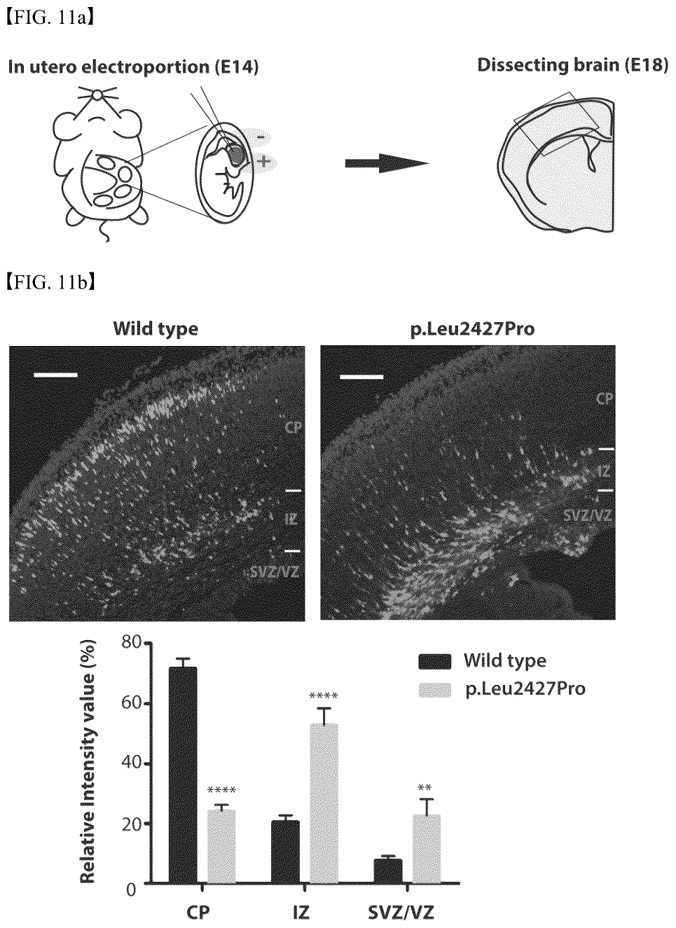

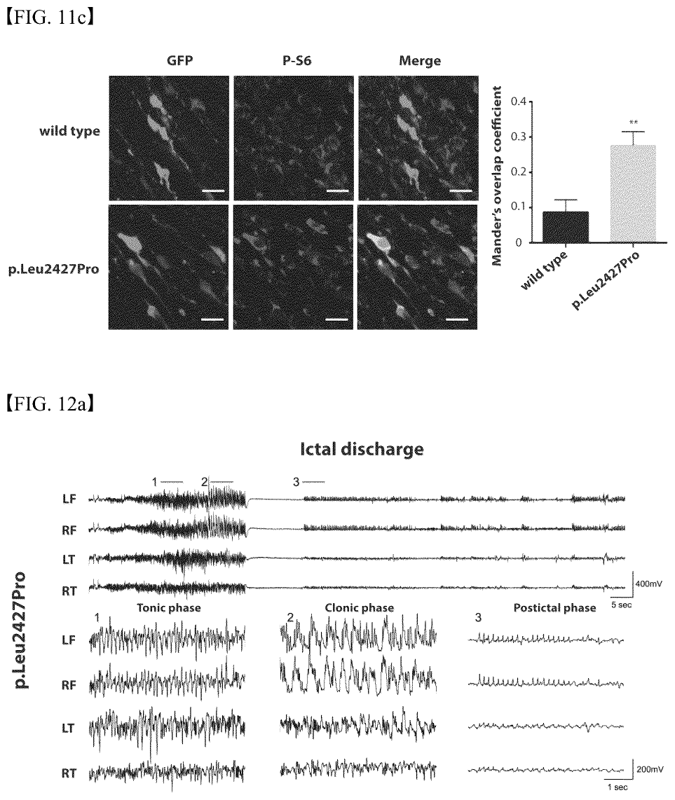

As shown in FIG. 11a, mTOR wild-type or p.Leu2427Pro constructs containing an IRES-GFP reporter were electroporated into the developing mouse cortex at embryonic day (E) 14, and measured for cortical radial migration and the S6 phosphorylation of GFP-positive neurons at E18.

It was observed, as shown in FIG. 11b, that brain sections expressing the mTOR mutant construct showed a significant decrease in GFP-positive cells in the cortical plate (CP) and an increase in GFP-positive cells in the intermediate zone (IZ), the subventricular zone (SVZ), and the ventricular zone (VZ), thereby indicating the disruption of cortical radial neuronal migration.

In addition, as shown in FIG. 11c, mTOR mutant expressing GFP-positive cells were observed to coexist with cells having an elevated level of S6 phosphorylation in brain sections. These findings suggest that the identified mTOR mutations cause the aberrant activation of mTOR kinase protein and the disruption of proper cortical development in vivo.

### Example 6: Identification of mTOR Hyperactivation-Induced Disease Phenotype in Animal Model

### Example 6-1. Identification of Spontaneous Seizure and Abnormal Neurons in Animal Model

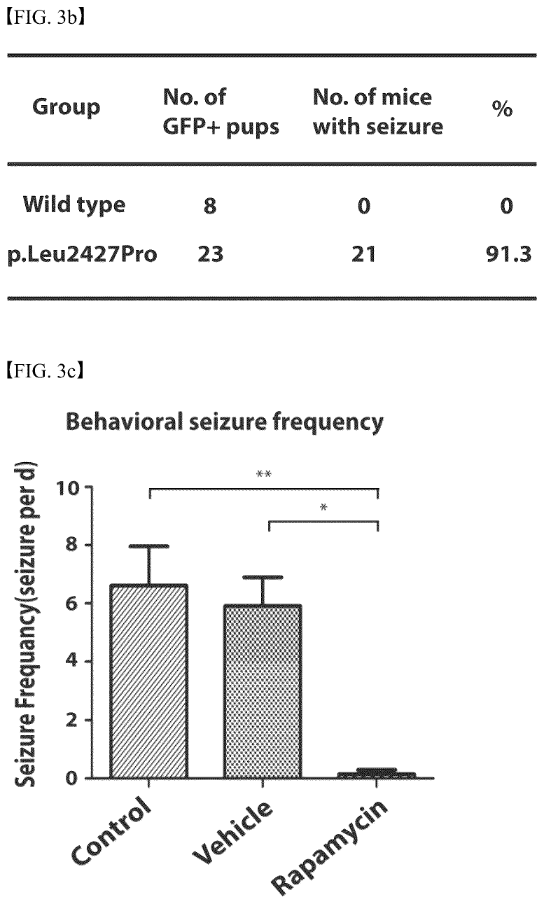

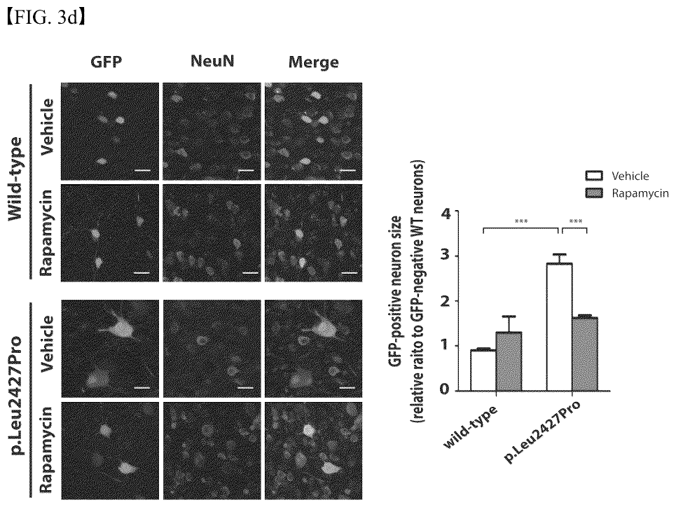

A determination was made to see whether the focal cortical expression of mTOR induces spontaneous seizures in mice after in utero electroporation. Subsequent to in utero electroporation at E14, properly delivered mice pups at birth that showed GFP signals on the electroporated cortical region were selected, as shown in FIG. 3a.

Thereafter, continuous video-electroencephalographic monitoring of the mice was performed starting 3 weeks after birth. After weaning, the mice were monitored by video-recoding for 12 hrs per day until tonic-clonic seizures were observed. Then, mice with seizures were monitored using video-electroencephalography for 6 hrs per day over two days to characterize the spontaneous seizures with epileptic discharge.

Briefly, after weaning (>3 weeks), seizures were observed only through video monitoring. Thereafter, electrodes for recoding electroencephalograms were surgically implanted. A total of five electrodes were located in the epidural layer: based on the bregma, two electrodes on the frontal lobes (AP+2.8 mm, ML+1.5 mm), two electrodes on the temporal lobes (AP-2.4 mm, ML+2.4 mm), and one electrode on the cerebellum. After more than 4 days of recovery from surgery, EGG signals were recorded between 6 p.m. and 2 a.m. for 2-5 days (6 hrs per day). Signals were amplified with an amplifier (GRASS model 9 EEG/Polysomnograph, GRASS technologies, USA), and analyzed with the pCLAMP program (Molecular Devices, USA). Alternatively, a RHD2000 amplifier and board (Intan Technologies, USA) and MATLAB EEGLAB (http://sccn.ucsd.edu/eeglab) were used for analysis.

For EGG analysis, 10-12 h continuous recording data was analyzed for interictal spike and nonconvulsive electrographic seizure counts. 1-min samples were selected from the data at standardized preset time points separated by exactly 1 h.

Each 1-min sample was assessed for the number of interictal epileptiform spikes and nonconvulsive electrographic seizures therein by an observer who was unaware of the treatment of the mice. Interictal spikes were defined as fast (<200 ms) epileptiform wave forms that occurred regularly and were at least twice the amplitude of the background activity. Nonconvulsive electrographic seizure episodes were counted when the EEG recording showed at least two connected spike-wave complexes (1-4 Hz) with amplitudes as at least twice as background, and were observed simultaneously in the majority of the four recording channels per mouse.

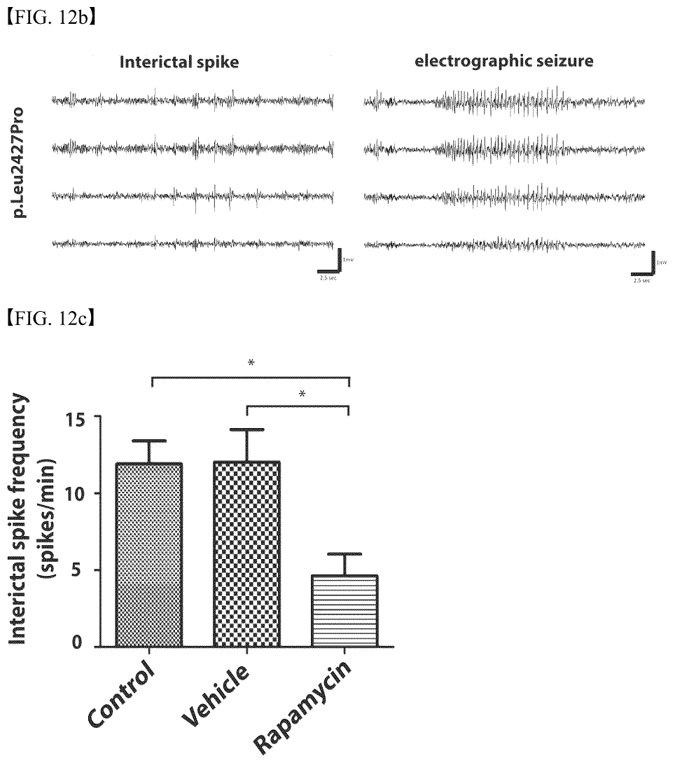

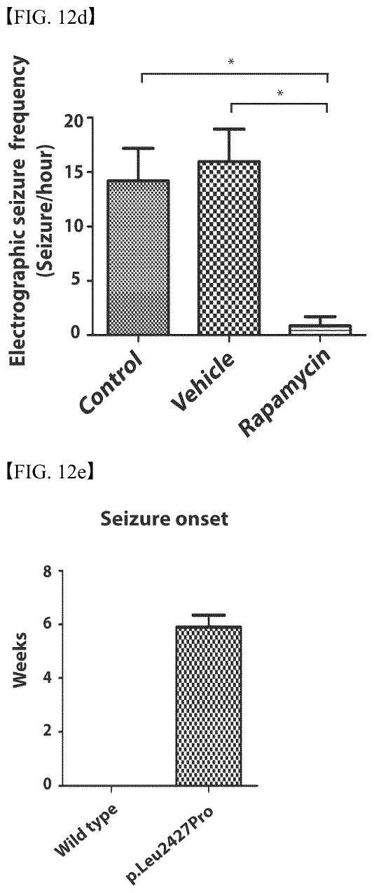

Surprisingly, as shown in FIGS. 3b and 12a, more than 90% of the mice expressing mTOR mutant constructs displayed spontaneous seizures with epileptic discharge, including high-voltage fast activity, high-voltage spikes and waves, and low-voltage fast activity. These mutant mice also showed interictal spikes and electrographic seizures (FIG. 12b). The mice in which spontaneous seizures were induced were observed to exhibit systemic tonic-clonic seizures consisting of a tonic phase, a clonic phase, and a postictal phase, similar to those found in FCDII patients. Further, brain waves are characterized by synchronized multi-waves of low-voltage, fast activity in the tonic phase, high-voltage standing waves in the clonic phase, and synchronized attenuated amplitudes in the postictal phase. However, neither spontaneous seizures with epileptic discharges nor electrographic seizure were observed in control mice electroporated with the mTOR wild-type construct, as shown in FIG. 3b.

The average seizure onset of p.Leu2427Pro mice started on average roughly 6 weeks after birth (FIG. 12e), which is approximately equivalent to that of human FCDII patients (˜4 years). The seizure frequency was about 6 events per day (FIG. 3c).

After the confirmation of seizures, investigation was made to see whether the mice electroporated with mTOR mutant constructs showed abnormal neuronal morphology, such as cytomegalic neurons.

It was observed that the soma sizes of GFP-positive neurons were greatly increased in affected cortical regions of electroporated mice carrying mTOR mutations (FIG. 3d).

### Example 6-2. Effect of Drug on Spontaneous Seizure and Abnormal Neuron

Animal models with spontaneous seizures or abnormal neurons were monitored after administration with rapamycin.

Briefly, rapamycin or everolimus (LC Labs, USA) was dissolved in 100% ethanol to give a 20 mg/ml stock solution and stored at −20° C. Immediately before injection, the stock solution was diluted in 5% polyethyleneglycol 400 and 5% Tween80 to yield final concentrations of 1 mg/ml rapamycin and 4% ethanol. Mice were injected with 1 to 10 mg/kg rapamycin for 2 weeks (10 mg/kg/d intraperitoneal injection, two weeks).

Rapamycin, as shown in FIGS. 3c, 12c and 12d, almost completely freed the mTOR mutant construct-carrying mice from spontaneous seizures, with a simultaneous dramatic decrease in the onset frequency of interictal spikes and nonconvulsive electrographic seizure.

Also, abnormal soma sizes of neurons were reduced in the animal model administered with rapamycin, as shown in FIG. 3d.

### Example 7: Identification of Mutation in Intractable Epilepsy Patient by Sequencing

Genomic DNA was extracted in substantially the same manner as that of Example 2 from samples from a total of 77 patients, listed in Examples 1 and 2, and subjected to hybrid capture sequencing and PCR-based amplicon sequencing. Of mutations found in both the sequencing analyses, those that met the selection standards (a total read depth of 100 or more, 3 or more mutated calls, and a mapping quality score of 30 or more) were detected in TSC1, TSC2, AKT3, and PIK3CA.

TSC1 c.64C>T (p.Arg22Trp), c.610C>T (p.Arg204Cys), c.2432G>T (p.Arg811Leu); TSC2 c.4639C>T (p.Val1547Ile); AKT3 c.740G>A (p.Arg247His), PIK3CA c.3052G>A (p.Asp1018Asn).

In eight of 51 patients negative for mTOR mutations, TSC1, TSC2, AKT3, and PIK3CA gene mutations were detected only in affected brain regions. Accordingly, 21 of a total of 77 intractable epilepsy patients were found to have mutations only in affected brain regions.

mTOR c.616C>T (p.Arg206Cys) mTOR c.1871G>A (p.Arg624His), c. 4348T>G (p.Tyr1450Asp), c.4447T>C (p.Cys1483Arg), c.5126G>A (p.Arg1709His), c.5930C>A (p.Thr1977Lys), c.6577C>T (p.Arg2193Cys), c.6644C>T (p.Ser2215Phe), and c.7280T>A (p.Leu2427Gln); TSC1 c.64C>T (p.Arg22Trp), c.610C>T (p.Arg204Cys), c.2432G>T (p.Arg811Leu); TSC2 c.4639C>T (p.Val1547Ile); AKT3 c.740G>A (p.Arg247His), PIK3CA c.3052G>A (p.Asp1018Asn).

TABLE 5

| | | | | | | | | PCR |
|---|---|---|---|---|---|---|---|---|
| | | | | | | | Hybrid | amplicon |
| | | | | | | | Capture | sequencing |
| | | | | | | | % | % |
| | Age at | | | | Modified | Modified | Mutated | Mutated |
| Disease/gender | surgery | phathology | MRI result | protein | nucleotide | Amino acid | allele | allele |
| FCD 4/female | 5 yr 2 m | Cortical dyslamination, | No abnormal | MTOR | c.7280T > C | p.Leu2427Pro | 7.94% | 12.6% |
| | | Dysmorphic neurons, | signal intensity | | | | | |
| | | consistent with FCDIIa | | | | | | |
| FCD 6/female | 5 yr | Cortical dyslamination, | No abnormal | MTOR | c.7280T > C | p.Leu2427Pro | 6.90% | 7.28% |
| | | Dysmorphic neurons, | signal intensity | | | | | |
| | | consistent with FCDIIa | | | | | | |
| FCD 64/female | 6 yr 9 m | Cortical dyslamination, | Cortical dysplasia | TSC1 | c.610C > T | p.Arg204Cys | 1.75% | 1.0% |
| | | Dysmorphic neurons, | involving left | | | | | |
| | | consistent with FCDIIa | fronto-parietal lobe | | | | | |
| HME 66/male | 2 yr 8 m | Cortical laminar | Rt. | PIK3CA | c.3052G > A | p.Asp1018Asn | 1.03% | 2.30% |
| | | disturbance with | hemimegalencephDaly | | | | | |
| | | large giant neurons | | | | | | |
| SWS 77/male | 11 m | Cortical dyslamination, | Difuse brain | MTOR | c.616C > T | p.Arg206Cys | 3.93% | 3.45% |
| | | Dysmorphic neurons, | atrophy, Right | | | | | |
| | | consistent with FCDIIa | hemisphere | | | | | |
| FCD 81/female | 12 yr | Cortical dyslamination, | No abnormal | TSC1 | c.64C > T | p.Arg22Trp | 2.81% | 2.0% |
| | | Dysmorphic neurons, | signal intensity | | | | | |
| | | consistent with FCDIIa | | | | | | |
| HS86/male | 13 yr 2 m | Hippocampal sclerosis | Suggestive of | AKT3 | c.740G > A | p.Arg247His | 1.72% | 10% |
| | | | HS, left. | | | | | |
| FCD 91/female | 7 yr 1 m | Cortical dyslamination, | Volume decrease of | MTOR | c.6577C > T | p.Arg2193Cys | 2.99% | 1.26% |
| | | Dysmorphic neurons, | the left cerebral | | | | | |
| | | consistent with FCDIIa | hemisphere and | | | | | |
| | | | multifocal lesions | | | | | |
| | | | in the WM | | | | | |
| FCD 94/female | 10 yr 3 m | Cortical dyslamination, | Subependymal | TSC2 | c.4639C > T | p.Val1547Ile | 1.19% | 1.55% |
| | | Dysmorphic neurons, | heterotopia, Rt | | | | | |
| | | consistent with FCDIIa | peri-trigone area | | | | | |
| FCD 98/male | 14 yr 3 m | Cortical dyslamination, | No abnormal | TSC1 | c.64C > T | p.Arg22Trp | 2.52% | 1.98% |
| | | Dysmorphic neurons, | signal intensity | | | | | |
| | | consistent with FCDIIa | | | | | | |
| FCD 104/male | 1 yr 2 m | Cortical dyslamination, | Cortical dysplasia | MTOR | c.1871G > A | p.Arg624His | 1.80% | 4.41% |
| | | Dysmorphic neurons, | involving right | | | | | |
| | | consistent with FCDIIa | precentral and | | | | | |
| | | | postcentral gyri, | | | | | |
| FCD 105/male | 3 yr 7 m | Cortical dyslamination, | No abnormal | MTOR | c.5126G > A | p.Arg1709His | 1.63% | 1.52% |
| | | Dysmorphic neurons, | signal intensity | | | | | |
| | | consistent with FCDIIa | | | | | | |
| FCD 107/female | 7 yr 3 m | Cortical dyslamination, | Cortical Dysplasia | MTOR | c.6644C > T | p.Ser2215Phe | 2.41% | 2.11% |
| | | dysmorphic neurons, | involving left | | | | | |
| | | balloon cells, | occipitoparietal | | | | | |
| | | consistent with FCDIIb | lobe and precentral | | | | | |
| | | | gyrus | | | | | |
| FCD 113/female | 10 yr | Cortical dyslamination, | Cortical dysplasia | MTOR | c.7280T > A | pLeu2427Gln | 3.05% | 5.11% |
| | | dysmorphic neurons, | involving left | | | | | |
| | | balloon cells, | occipital and | | | | | |
| | | consistent with FCDIIb | parietal lobe | | | | | |
| FCD 116/male | 7 yr 9 m | Cortical dyslamination, | Cortical dysplasia | MTOR | c.5930C > A | p.Thr1977Lys | 3.25% | 2.93% |
| | | dysmorphic neurons, | involving left | | | | | |
| | | balloon cells, | superior frontal | | | | | |
| | | consistent with FCDIIb | gyrus | | | | | |
| FCD 121/male | 11 m | Cortical dyslamination, | Cortical dysplasia | MTOR | c.4348T > G | p.Tyr1450Asp | 2.64% | 3.76% |
| | | dysmorphic neurons, | involving entire right | | | | | |
| | | balloon cells, | lobe and left superior/ | | | | | |
| | | consistent with FCDIIb | middle frontal gyrus | | | | | |
| FCD 123/female | 12 yr 4 m | Cortical dyslamination, | Cortical Dysplasia | TSC1 | c.64C > T | p.Arg22Trp | 2.21% | 1.37% |
| | | dysmorphic neurons, | involving right | | | | | |
| | | balloon cells, | frontal lobe | | | | | |
| | | consistent with FCDIIb | | | | | | |
| FCD 128/female | 4 yr 4 m | Cortical dyslamination, | Cortical dysplasia, | MTOR | c.4447T > C | p.Cys1483Arg | 6.38% | 9.77% |
| | | dysmorphic neurons, | right frontal lobe | | | | | |
| | | balloon cells, | | | | | | |
| | | consistent with FCDIIb | | | | | | |
| HME141female | 1 yr 9 m | Cortical laminar | Lt. | TSC1 | c.2432G > T | p.Arg811Leu | 1.03% | 1.68% |
| | | disturbance with | hemimegalencephaly | | | | | |
| | | large giant neurons | | | | | | |
| FCD 143/female | 2 yr 10 m | Cortical dyslamination, | No abnormal | MTOR | c.6644C > T | p.Ser2215Phe | 2.82% | 2.33% |
| | | dysmorphic neurons, | signal intensity | | | | | |
| | | balloon cells, | | | | | | |
| | | consistent with FCDIIb | | | | | | |
| FCD 145/female | 4 yr 1 m | Cortical dyslamination, | Cortical dysplasia | MTOR | c.5930C > A | p.Thr1977Lys | 1.46% | 1.51% |
| | | dysmorphic neurons, | involving left | | | | | |
| | | balloon cells, | precentral gyrus | | | | | |
| | | consistent with FCDIIb | | | | | | |

### Example 8: In Vivo Analysis of mTOR Hyperactivation

8-1. Mutagenesis and Construction of TSC1, TSC2, and AKT3 mutant constructs pcDNA3 carrying HA-tagged wild-type TSC1, TSC2 or AKT3 (pcDNA3 HA-tagged wild-type TSC1, TSC2, AKT3 construct) was purchased from Addgene (USA). The construct was used to generate mutant vectors with a QuikChange II site-directed mutagenesis kit (200523, Stratagene, USA).

pcDNA3 carrying HA-tagged wild-type TSC1, TSC2 or AKT3 (pcDNA3 HA-tagged wild-type TSC1, TSC2, AKT3 construct) was purchased from Addgene (USA). In the pcDNA3 TSC1, TSC2, AKT3 wild-type vector, the mutagenesis of TSC-1 R22W and R204C was achieved by use of TSC-1 R22W-F and R22W-R primers for R22W mutagenesis and by use of TSC-1 R204C-F and R204C-R primers for R204C mutagenesis. TSC-2 V1547I-F and V1547I-R primers were used for TSC-2 V15471 mutagenesis in the pcDNA3 TSC2 wild-type vector. For the mutagenesis of AKT3 R247H in the pcDNA3 AKT3 wild-type vector, R247H-F and R247H-R primer were designed.

A QuikChange II site-directed mutagenesis kit (200523, Stratagene, USA) was used to create point mutations. Because each primer has a site-specific point mutation sequence, a mutation is induced in copies of the sequence upon PCR. Primers useful for the mutagenesis are listed in Table 6, below.

TABLE 6

| | Location n of | | | | |
|---|---|---|---|---|---|
| gene | Modification | | Primer | | SEQ ID NO |
| TSC-1 | C64T | R22W | TSC-1 R22W-F | gtcacgtcgtcccacacacccagcatg | 29 |
| | | | TSC-1 R22W-R | catgctgggtgtgtgggacgacgtgac | 30 |
| | C610T | R204C | TSC-1 R204C-F | ctttcatactgtaatgagaacacaaaaaggagacgaagttgca | 31 |
| | | | TSC-1 R204C-R | tgcaacttcgtctcctttttgtgttctcattacagtatgaaag | 32 |
| TSC-2 | G4639A | V1547I | TSC-2 V15471-F | tctccaacatacaggatggcgatcttgtgggtg | 33 |
| | | | TSC-2 V1547I-R | cacccacaagatcgccatcctgtatgttggaga | 34 |
| AKT3 | G740A | R247H | AKT3 R247H-F | caccatagaaacgtgtgtggtcctcagagaacacc | 35 |
| | | | AKT3 R247H-R | ggtgttctctgaggaccacacacgtttctatggtg | 36 |

### 8-2. Cell Culture, Transfection, and Western Blot

In order to examine whether TSC-1, TSC-2 or AKT3 mutation causes the aberrant activation of mTOR, wild-type or mutant vectors were transfected into HEK293T and the phosphorylation of SK6, widely known as an mTOR gene marker, was analyzed by Western blotting.

Briefly, HEK293T cells (Thermo Scientific) were cultured in DMEM (Dulbecco's Modified Eagle's Medium) supplemented with 10% FBS at 37° C. and 5% CO₂. The cells were transfected with empty flag-tagged vector, HA-tagged TSC1 wild-type, HA-tagged TSC2 wild-type, HA-tagged AKT3 wild-type, HA-tagged TSC1 mutant, HA-tagged TSC2 mutant and HA-tagged AKT3 mutant, respectively, using jetPRIME transfection reagent (Polyplus, France).

The cells were serum-starved with 0.1% FBS in DMEM for 24 hours after transfection and then incubated at 37° C. and 5% CO₂ in PBS containing 1 mM MgCl₂ and CaCl₂) for 1 hour. The cells were lysed with PBS containing 1% Triton X-100 and Halt protease and phosphatase inhibitor cocktail (78440, Thermo Scientific, USA). Proteins were resolved by SDS-PAGE and transferred to PVDF membranes (Millipore, USA). The membranes were blocked with 3% BSA in TBS containing 0.1% Tween 20 (TBST). They were washed 4 times with TBST. The membranes were incubated overnight with primary antibodies including a 1/1000 dilution of anti-phospho-S6 ribosomal protein (5364, Cell Signaling Technology, USA), anti-S6 ribosomal protein (2217, Cell Signaling Technology, USA), and anti-flag M2 (8164, Cell Signaling Technology, USA) in TBST at 4° C., respectively. After incubation, the membranes were washed 4 times with TBST. They were incubated with a 1/5000 dilution of HRP-linked anti-rabbit or anti-mouse secondary antibodies (7074, Cell Signaling Technology, USA) for 2 hours at room temperature. After washing with TBST, immunodetection was performed using ECL reaction reagents.

### 8-3. Treatment of Mutant-Expressing Cells with Rapamycin and Western Blot

After treatment with rapamycin, the mutant-expressing cells of Example 8-2 were monitored for S6K phosphorylation.

Briefly, HEK293T cells were transfected with mTOR, TSC1, TSC2, or AKT3 mutants in the same manner as in Example 8-2. The transfected cells were starved for 24 hrs with empty DMEM and incubated at 37° C. and 5% CO₂ for 1 hr with rapamycin in PBS containing 1 mM MgCl₂ and CaCl₂), followed by Western blotting in the same manner as in Example 2-2.

### 8-4. Experiment Data

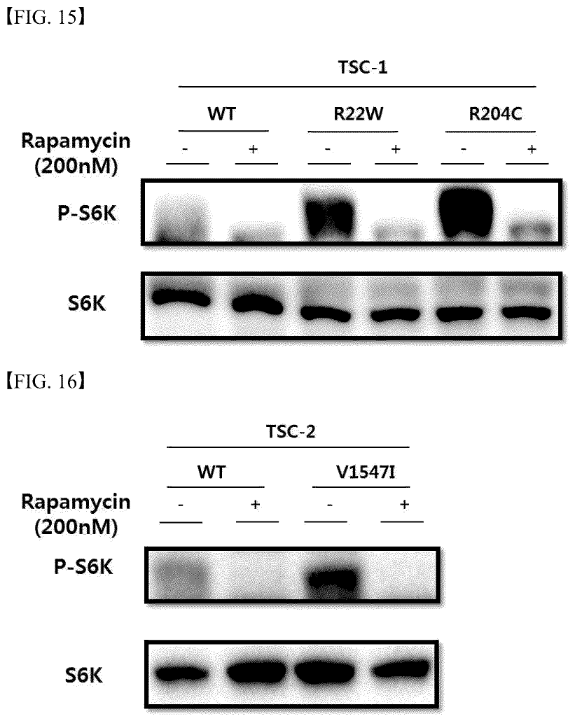

In order to examine whether the p.Arg22Trp and p.Arg204Cys mutations of TSC-1, the p. Val1547Ile mutation of TSC-2, or the p.Arg247His mutation of AKT3 induces mTOR activation, HEK293T cells were transfected with vectors carrying TSC1, TSC2, and AKT3 wild-type and mutants, and S6K phosphorylation, a well-known index for mTOR mutation, was monitored via Western blotting. The mutant-expressing cells were treated with rapamycin before monitoring the phosphorylation of S6K, as described in Examples 8-2 and 8-3. The results are depicted in FIGS. 15 to 17, and can be described for individual mutant genes) as follows:

### (1) In Vitro Activity of TSC-1 Mutant

As can be seen in FIG. 15, S6K phosphorylation was increased in cells expressing mutant TSC-1, and reduced by rapamycin treatment.

### (2) In Vitro Activity of TSC-2 Mutant

As can be seen in FIG. 16, S6K phosphorylation was increased in cells expressing mutant TSC-2, and reduced by rapamycin treatment.

### (3) In Vitro Activity of AKT3 Mutant

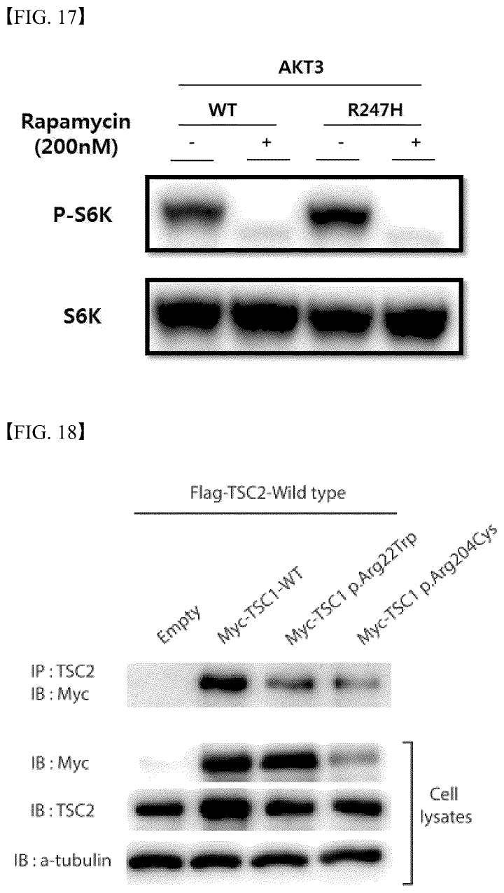

As can be seen in FIG. 17, S6K phosphorylation was increased in cells expressing mutant AKT3, and reduced by rapamycin treatment.

### Example 9: Activation of mTOR Pathway by TSC1 and TSC2 Mutants

### 9-1: Immunoprecipitation Assay

To examine whether mutations in TSC1 and TSC2 disrupt the formation of the TSC complex, immunoprecipitation assays were conducted on wild-type and mutant TSC1 or TSC2-expressing HEK293T cells. In this regard, TSC1 and TSC2 mutant proteins prepared in the same manner as in Example 8-3 were incubated overnight with an anti-TSC2 antibody (3990, Cell signaling Technology, USA) or an anti-myc antibody (2276, cell signaling technology, USA), and then with protein A+G magnetic beads for 2 hrs. After washing with PBS containing 1% Triton-X100, the beads were incubated in an SDS buffer at 37° C. for 10 min. After being eluted, proteins were resolved on SDS/PAGE gel and transferred to a PVDF membrane. Immunoblotting was performed in the same manner as in Example 2-3.

The results are depicted in FIG. 18. It was found that the TSC-1 p.Arg22Trp and p.Arg204Cys mutations located near the TSC2 binding domain strongly inhibited TSC1 binding to TSC2. These data imply that the TSC1 mutant disrupted the formation of the TSC complex, leading to the hyperactivation of mTOR kinase.

### 9-2: GTP-Agarose Pull Down Assay

Cells were harvested in a lysis buffer (20 mM Tris-HCl pH: 7.5, 5 mM MgCl₂, 2 mM PMSF, 20 μg/mL leupeptin, 10 μg/mL aprotinin, 150 mM NaCl and 0.1% Triton X-100) and then lysed by sonication for 15 sec. The cell lysates were centrifuged at 4° C. and 13,000 g. The supernatant was separated and incubated with 100 μl of GTP-agarose beads (Sigma-Aldrich, cat no. G9768) at 4° C. for 30 min. The beads were washed with a lysis buffer and again incubated overnight with the supernatant. GTP-bound proteins were extracted and visualized by immunoblots.

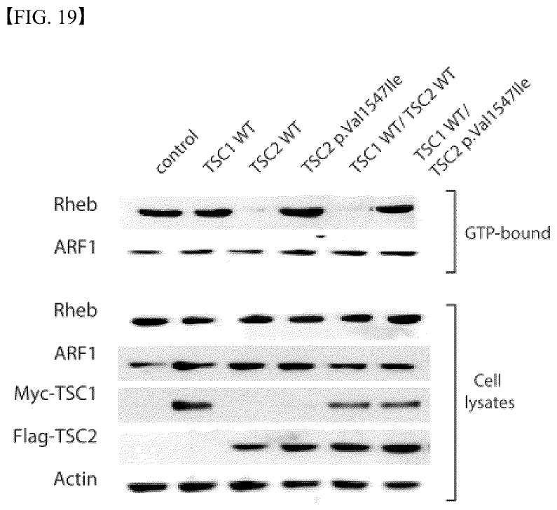

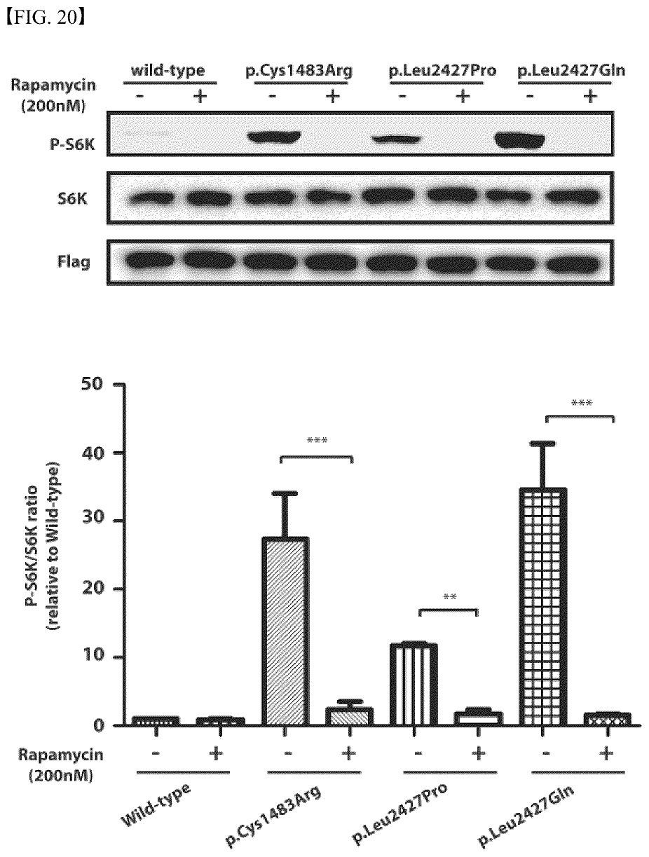

The expression of GTP-bound Rheb protein was found to decrease in wild-type TSC2-expressing cells, but not in TSC2 p.Val1547Ile mutant-expressing cells because the GAP (GTPase activating protein) activity of TSC2 was decreased (FIG. 19 ( [symbol] FIG. 2)), suggesting that the TSC2 mutant reduces the function of the GAP domain, thereby activating the mTOR pathway.

### Example 10: Monitoring of S6K Phosphorylation Level in Drug-Treated, Mutant mTOR-Expressing Cells

### 10-1. Mutant mTOR-Expressing Cell

Mutant mTOR-expressing cells were treated with drugs (rapamycin, everolimus, compounds of Chemical Formulas 1 to 4) and monitored for S6K phosphorylation level.

In this regard, HEK293T cells were transfected with the mutants in the same manners as in Examples 8-2 and 8-3, serum-starved for 24 hrs with 01. % FBS in DMEM, and incubated at 37° C. and 5% CO₂ for 1 hr with 1 mM MgCl₂ and CaCl₂) in PBS before treatment with rapamycin, everolimus, or the compounds of Chemical Formulas 1 to 4 (Torin1, INK128, AZD8055, GSK2126458): Torin was purchased from TOCRIS; INK128, AZD8055 and GSK2126458 were from Selleckchem; and everolimus was from LC laboratory. Subsequently, Western blotting was performed in the same manner as in Example 2-4.

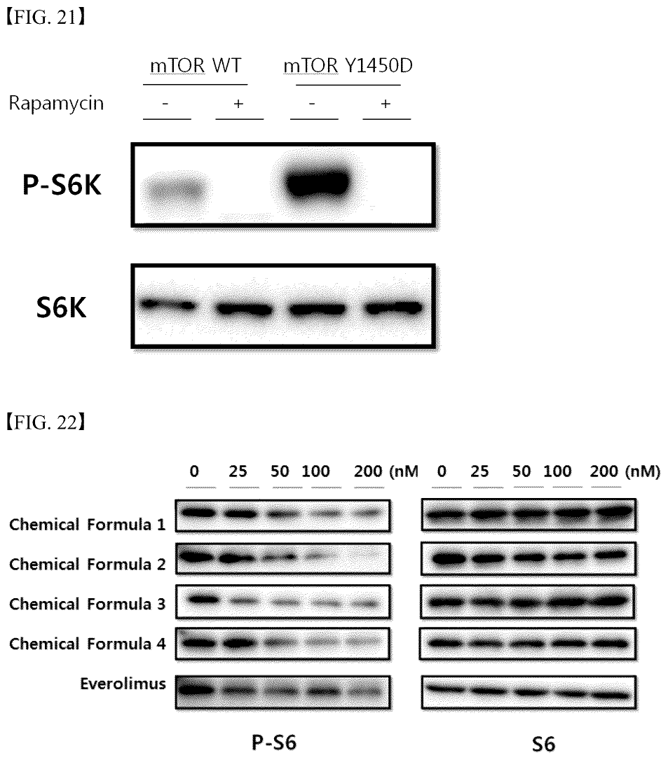

As is understood from the data of FIGS. 20 and 21, S6K phosphorylation was inhibited in mutant mTOR-expressing cells. Briefly, FIG. 20 shows levels of S6K phosphorylation in cells respectively expressing the mOTR mutants C1483R, L2427P and L2427Q after rapamycin treatment. FIG. 21 shows levels of S6K phosphorylation in mTOR mutation Y1450D-expressing cells after rapamycin treatment.

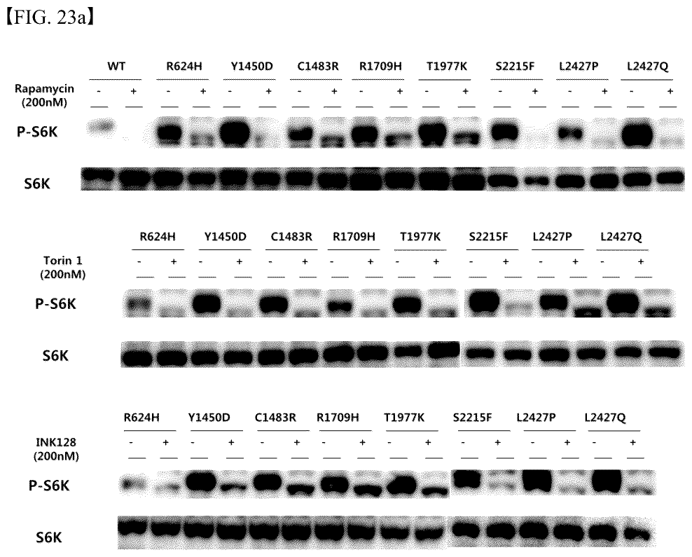

FIG. 22 shows levels of S6 in mTOR mutation L2427P-expressing cells after treatment with 0, 25, 50, 100, and 200 nM of rapamycin.

S6K phosphorylation was monitored following treatment with everolimus, or the compounds of Chemical Formulas 1 to 4. As can be seen in FIG. 22, the phosphorylation of S6 in mutant mTOR-expressing cells was inhibited by everolimus or the compounds of Chemical Formulas 1 to 4. A distinct decrease in S6 phosphorylation was apparent at a concentration of 50 nM or higher.

### Example 10-2. Change of S6K Phosphorylation with Various mTOR Inhibitors

Cells expressing various mTOR mutations were treated in the same manner as in Example 9-1 with rapamycin, everolimus, or the compounds of Chemical Formulas 1 to 4, and monitored for S6K phosphorylation. The mTOR variants were R624H, Y1450D, C1483R, R1709H, Y1977K, S2215F, L2427P, and L2427Q.

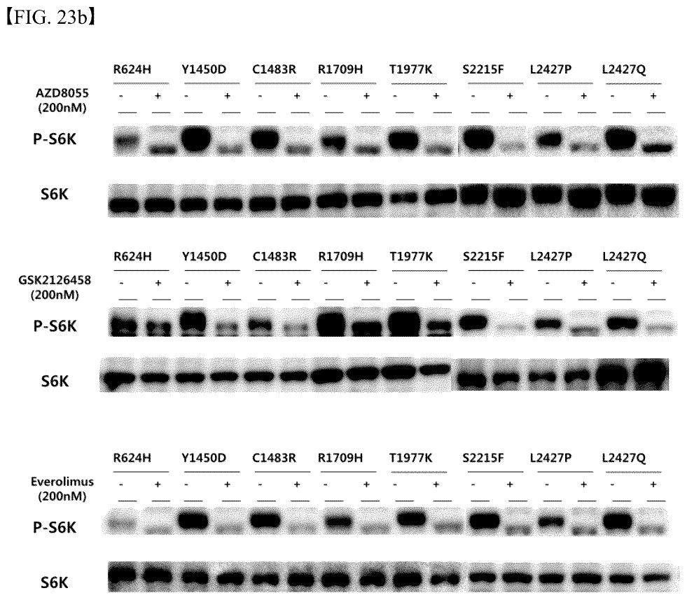

Briefly, the mutant mTOR-expressing cells were monitored for S6K phosphorylation level after treatment with everolimus or the compounds of Chemical Formulas 1 to 4. The results are depicted in FIGS. 23a and 23b. As shown, the phosphorylation of S6K in all the mutant mTOR-expressing cells was inhibited by everolimus or the compounds of Chemical Formulas 1 to 4.

### Example 11: Monitoring of S6K Phosphorylation in TSC1 or TSC2 Mutant-Expressing Cells Treated with Drugs

HEK293T cells were transfected with TSC1 or TSC2 mutants in the same manner as in Example 8, serum-starved for 24 hrs with 01. % FBS in DMEM, and incubated at 37° C. and 5% CO₂ for 1 hr with 1 mM MgCl₂ and CaCl₂) in PBS.

Thereafter, the cells were treated with rapamycin, everolimus, or the compounds of Chemical Formulas 1 to 4 (Torin1, INK128, AZD8055, GSK2126458): Torin was purchased from Tocris; INK128, AZD8055 and GSK2126458 were from Selleckchem; and everolimus was from LC laboratory. Subsequently, Western blotting was performed in the same manner as in Example 10.

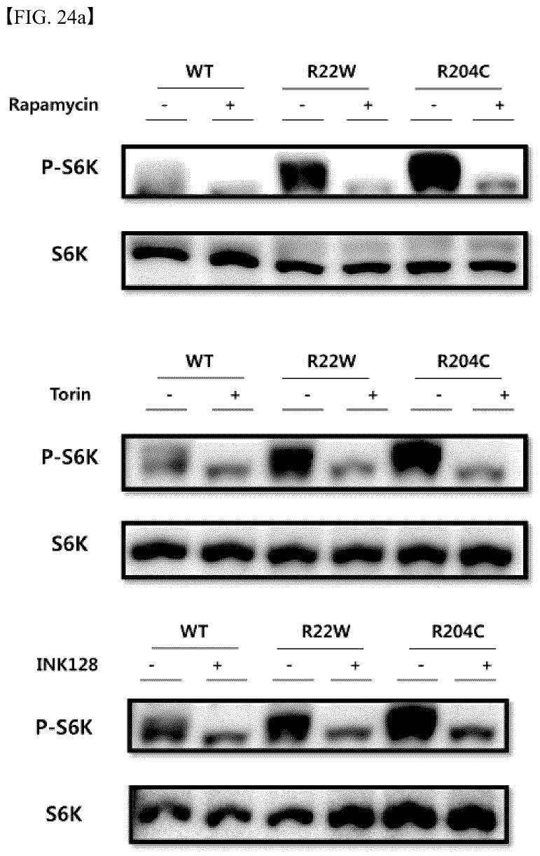

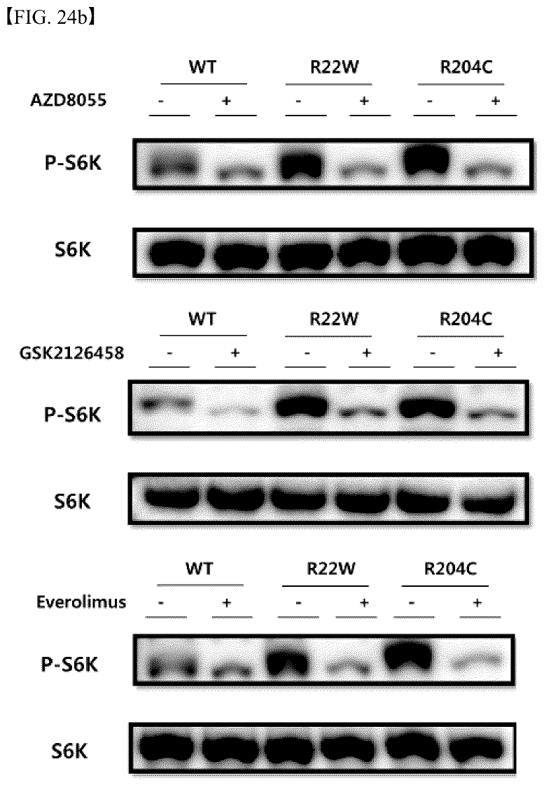

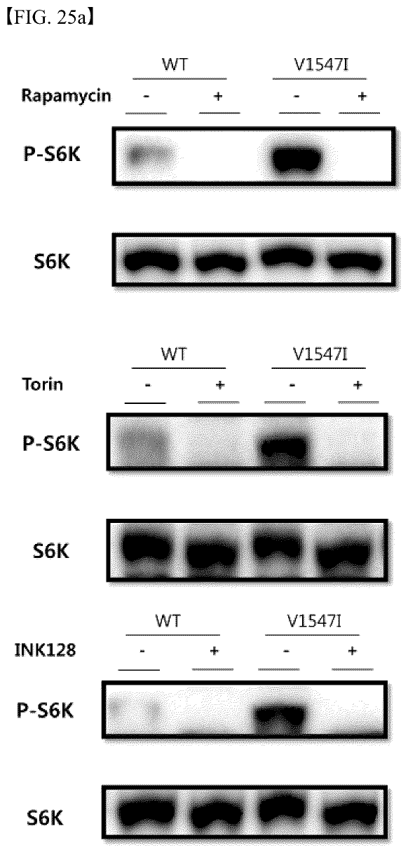

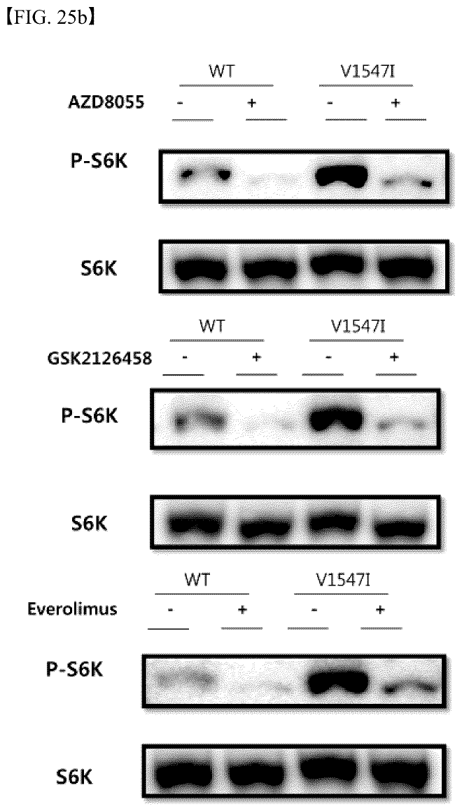

The TSC1 or TSC2 mutant-expressing cells were treated with rapamycin and monitored for S6K phosphorylation. The results are depicted in FIGS. 24a and 24b for the TSC1 mutant and in FIGS. 25a and 25b for the TSC2 mutant.

As is understood from the data of FIGS. 24a, 24b, 25a and 25b, S6K phosphorylation in TSC1 or TSC2 mutant-expressing cells was inhibited by rapamycin. Also, S6K phosphorylation was monitored in the TSC1 or TSC2-expressing cells following treatment with everolimus or the compounds of Chemical Formulas 1 to 4. As can be seen, everolimus or compounds of Chemical Formulas 1 to 4 was found to inhibit the phosphorylation of S6K in TSC1 or TSC2 mutant-expressing cells.

### Example 12: Immunostaining of Brain Tissue Section of FCD Patient

To determine whether the affected brains of FCDII patients carrying mutations are associated with mTOR hyperactivation, immunostaining was performed for S6 phosphorylation and NeuN (a neuronal marker) in brain tissue sections obtained from FCD patients carrying the p.Leu2427Pro mutation.

Non-malformations of cortical development (non-MCD) brain specimens were collected in the operating room from the tumor-free margin of individual patients with glioblastoma as part of a planned resection, which was pathologically conformed as a normal brain without tumors. Surgical tissue blocks were fixed overnight in freshly prepared phosphate-buffered (PB) 4% paraformaldehyde, cryoprotected overnight in 20% buffered sucrose, and prepared into gelatin-embedded tissues blocks (7.5% gelatin in 10% sucrose/PB) before storage at −80° C. Cryostat-cut sections (10 um thick) were collected and placed on glass slides. FFPE slides were deparaffinized and rehydrated to remove paraffin. Then, a heat-induced retrieval process was performed on the deparaffinized FFPE slides using a citrate buffer to enhance the staining intensity of the antibodies. The slides were then blocked in PBS-GT (0.2% gelatin and 0.2% Triton X-100 in PBS) at room temperature for 1 hr before staining with the following antibodies: rabbit antibody to phosphorylated S6 ribosomal protein (Ser240/Ser244) (1:100 dilution; 5364, Cell signaling Technology) and mouse antibody to NeuN (1:100 dilution; MAB377, Millipore). Subsequently, samples were washed in PBS and stained with the following secondary antibodies: Alexa Fluor 555-conjugated goat antibody to mouse (1:200 dilution; A21422, Invitrogen) and Alexa Fluor 488-conjugated goat antibody to rabbit (1:200 dilution; A11008, Invitrogen).

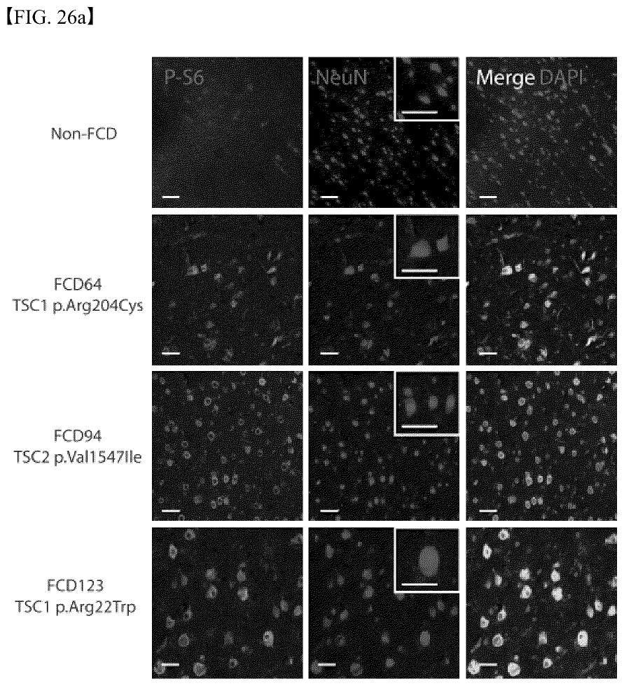

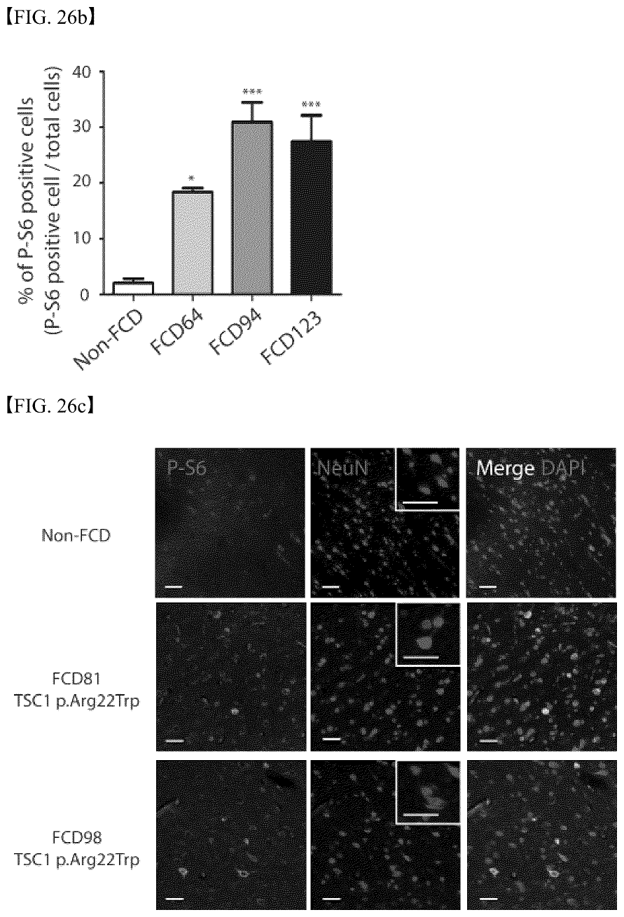

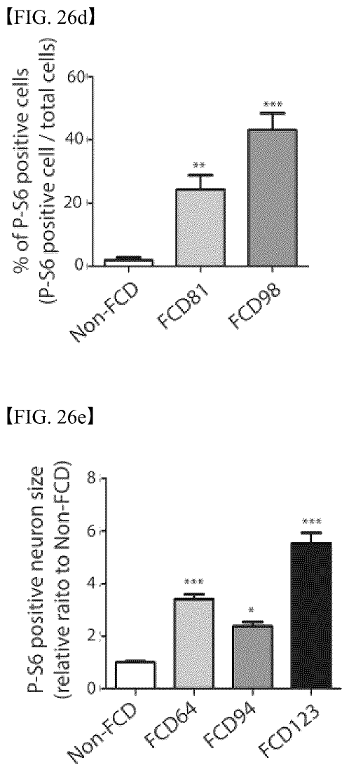

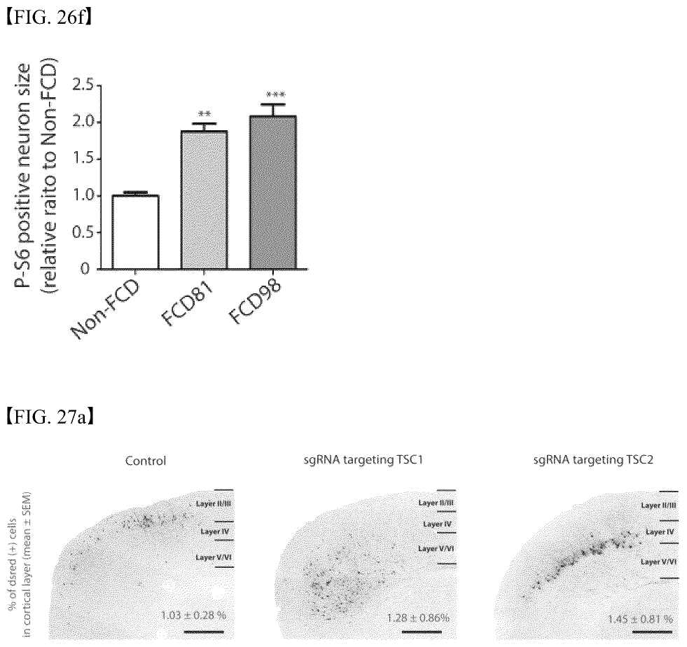

DAPI, included in a mounting solution (P36931, Life technology), was used for nuclear staining. Images were acquired using a Leica DMI3000 B inverted microscope. Cells positive for NeuN were counted using a 10× objective lens; 4-5 fields were acquired per subject within neuron-rich regions, and 100 or more cells were scored per region. The number of DAPI-positive cells represents total cell counts. Neuronal cell sizes were measured in NeuN-positive cells using the automated counting protocol of ImageJ software (http://rsbweb.nih.gov/ij/). The experimental results are given in FIGS. 26a to 26f.

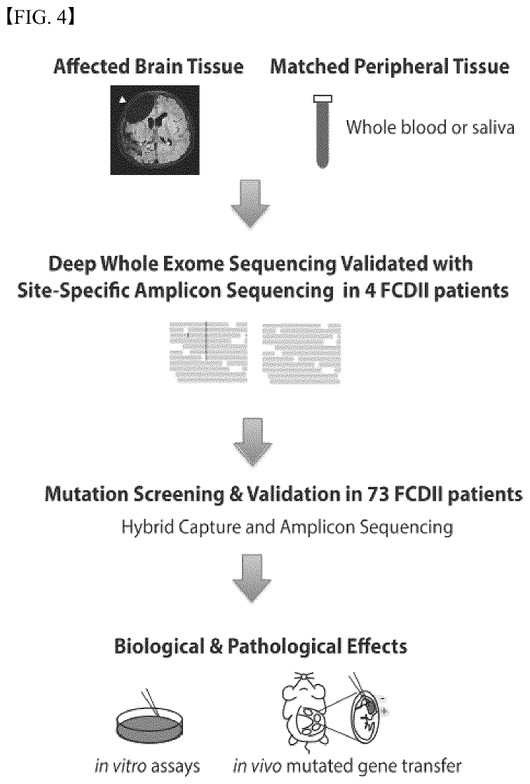

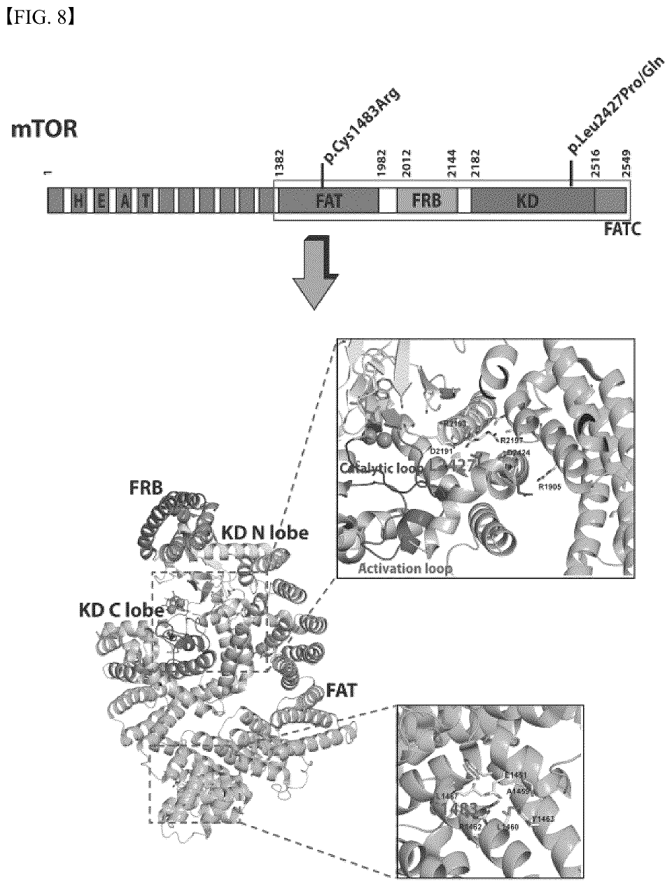

As seen in FIGS. 2a to 26f, the results showed a marked increase in the number of neuronal cells positive for phosphorylated S6 in FCD patients #64, 81, 94, 98, and 128 carrying TSC1 or TSC2 mutations, but not in non-FCD brains. In addition, the results revealed a robust increase in the number of neuronal cells that were positive for phosphorylated S6 in patients carrying TS (′/and TS (′2 mutations compared with non-FCD brains, as shown in FIGS. 26b and 26d, and in the soma size of phosphorylated S6-positive neurons in the pathological samples, as shown in FIGS. 26e and 26f.

### Example 13: Construction of TSC1 or TSC2 Mouse Model

### 13-1: Construction of TSC1- or TSC2-Targeting CRISPR/Cas9 Vector

A commercially available pX330 plasmid (Addgene, #42230) was used as an initial template. Using a QuikChange site-directed mutagenesis kit (Stratagene, La Jolla, CA), the sgRNA (single guide ribonucleotide) cloning site was modified to change the restriction enzyme recognition site BbsI (GAAGAC) to BsaI (GGTCTC). Subsequently, sgRNAs, targeting respective TSC1 and TSC2 genes, was inserted, the sequences of which are as follows.

TSC1:
(SEQ ID NO. 37)
5′-TGCTGGACTCCTCCACACTG-3′
TSC2:
(SEQ ID NO. 38)
5′-AATCCCAGGTGTGCAGAAGG-3′

To generate a plasmid carrying an mCherry fluorescent reporter (U6-sgRNA-Cas9-IRES-mCherry), IRES-mCherry was amplified PCR with the IRES3-mCherry-CL plasmid serving as a template. After PCR amplification, the IRES-mCherry was inserted between the Cas9 sequence and the NLS of px330.

### Example 13-2. Construction of Mouse Model

First, the TSC1- or TSC2-targeting U6-sgRNA-Cas9-IRES-mCherry plasmid, prepared in Example 19-1, was diluted at a ratio of 3:1 with pCAG-Dsred (Addgene #11151) to enhance red signals. Timed pregnant mice (E14) (Damul Science) were anesthetized with isoflurane (0.4 L/min of oxygen and isoflurane vaporizer gauge 3 during surgery operation). The uterine horns were exposed, and the lateral ventricle of each embryo was injected using pulled glass capillaries with 2 μg/ml of Fast Green (F7252, Sigma, USA) combined with 2-3 μg of the mixture of the two plasmids. Plasmids were electroporated on the head of the embryo by discharging 50 V with the ECM830 eletroporator (BTX-Harvard apparatus) in five electric pulses of 100 ms at 900-ms intervals. After delivery, selection was made of the mouse pups that exhibited fluorescence, screened using a flashlight (Electron Microscopy Science, USA).

### 13-3: Assay for Neuronal Migration in TSC1 or TSC2 Mouse Model

Brains were harvested from adult mice (P>56) prepared in Example 13-2, fixed overnight in freshly prepared phosphate-buffered (PB) 4% paraformaldehyde, cryoprotected overnight in 30% buffered sucrose, and prepared into gelatin-embedded tissue blocks (7.5% gelatin in 10% sucrose/PB) before storage at −80° C.

Cryostat-cut sections (30 um thick) were collected and placed on glass slides. DAPI, included in a mounting solution (P36931, Life technology), was used for nuclear staining. Images were acquired using a Zeiss LSM780 confocal microscope. Fluorescence intensities, reflecting the distribution of electroporated cells within the cortex, were converted into gray values and measured from layer II/III to layer V/VI using ImageJ software (http://rsbweb.nih.gov/ij/).

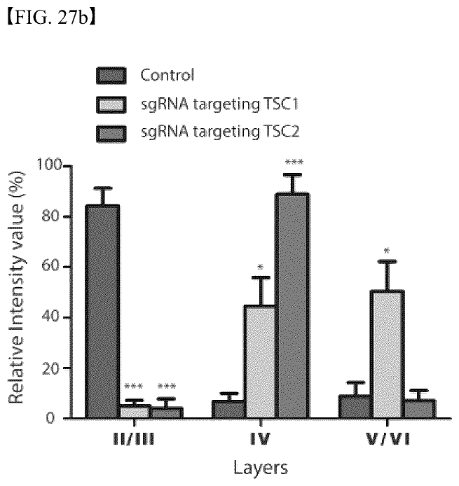

As can be seen in FIGS. 27a and 27b, dsRed-positive neurons in brain tissue sections of the TSC mouse models were decreased in the cortical layer II/III and increased in the layer II and layer V/VI, indicating that the radial migration of cortical neurons was prevented.

### 13-4: Video-Electroencephalography Monitoring

After weaning (>3 weeks), the mice were observed for seizures through video monitoring. Thereafter, electrodes for recoding electroencephalograms were surgically implanted. A total of five electrodes were located in the epidural layer: based on the bregma, two electrodes on the frontal lobes (AP+2.8 mm, ML+1.5 mm), two electrodes on the temporal lobes (AP-2.4 mm, ML+2.4 mm), and one electrode on the cerebellum. After 4 days of recovery from surgery, EGG signals were recorded for 6 hrs per day between 6 p.m. and 2 a.m. over 2-5 days. Signals were amplified using an RHD2000 amplifier and board (Intan Technologies, USA) and analyzed using MATLAB EEGLAB (http://sccn.ucsd.edu/eeglab).

The mice whose brains exhibited local TSC1- or TSC2-knockout resulting from use of the CRISPR/Cas9 plasmid displayed spontaneous seizures with epileptic discharges, including high-voltage fast activity, high-voltage spikes and waves, and low-voltage fast activity. The mice in which spontaneous seizures were induced were observed to exhibit systemic tonic-clonic seizures consisting of a tonic phase, a clonic phase, and a postictal phase, similar to those found in FCDII patients. Further, brain waves are characterized by synchronized multi-waves of low-voltage, fast activity in the tonic phase, high-voltage standing waves in the clonic phase, and synchronized attenuated amplitudes in the postictal phase. The seizure frequency was about 10 events per day.

### 13-5: Soma Size of Neurons in TSC1 or TSC2 Mouse Model

After EGG monitoring, the brains of the mice were excised by perfusion fixation using a phosphate-buffered (PB) 4% paraformaldehyde solution with the aid of a Masterflex compact peristaltic pump (Cole-Parmer international, USA). The brains were fixed in a freshly prepared phosphate-buffered (PB) 4% paraformaldehyde solution, cryoprotected overnight in 30% buffered sucrose, and prepared into gelatin-embedded tissue blocks (7.5% gelatin in 10% sucrose/PB) before storage at −80° C. Cryostat-cut sections (30 um thick) were collected, placed on glass slides, and blocked in PBS-GT (0.2% gelatin and 0.2% Triton X-100 in PBS) at room temperature for 1 hr before staining with the following antibodies: mouse antibody to NeuN (1:500 dilution; MAB377, Millipore). Subsequently, samples were washed in PBS and stained with the following secondary antibody: Alexa Fluor 488-conjugated goat antibody to mouse (1:200 dilution; A11008, Invitrogen). DAPI included in a mounting solution (P36931, Life technology) was used for nuclear staining. Images were acquired using a Zeiss LSM780 confocal microscope. Neuronal cell sizes were measured using ImageJ software (http://rsbweb.nih.gov/ij/).

Neurons were found to significantly increase in soma size for the mice with the local TSC1- or TSC2-knockout by use of the CRISPR/Cas9 plasmid, compared to normal neurons, but remained unchanged in size for the mice into which the plasmid was electroporated without sgRNA, which was consistent with the dysmorphic neurons of patients with Malformations of Cortical Developments.

### Example 14: Effect of Drug on Spontaneous Seizure in TSC2 Mouse Model

The animal models exhibiting spontaneous seizures were monitored after administration with rapamycin. Briefly, rapamycin (LC Labs, USA) was dissolved in 100% ethanol to give a 20 mg/ml stock solution, and stored at −20° C. Immediately before injection, the stock solution was diluted in 5% polyethyleneglycol 400 and 5% Tween80 to final concentrations of 1 mg/ml rapamycin and 4% ethanol. Mice were injected with 1 to 10 mg/kg rapamycin for 2 weeks (10 mg/kg/d intraperitoneal injection).

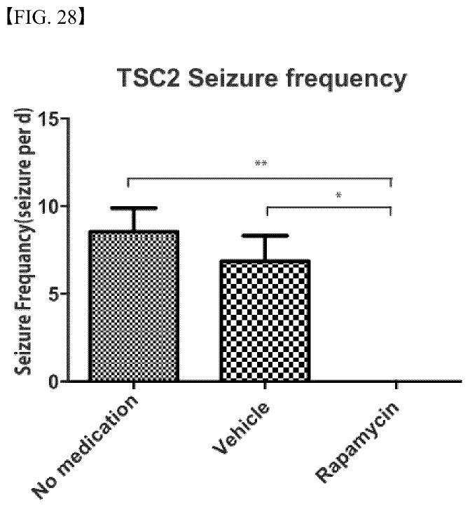

Rapamycin, as shown in FIG. 28, almost completely freed the animal models from spontaneous seizures.

What is claimed is:

1. A method for treating Focal Cortical Dysplasia (FCD) type II in a subject, comprising administering to the subject an effective amount of an mTOR inhibitor, wherein the subject is characterized by mTOR pathway hyperactivation and dysmorphic neurons in cerebral cortex.

2. The method of claim 1, wherein the subject is characterized by disrupted cortical lamination.

3. The method of claim 1, wherein the mTOR inhibitor is Rapamycin, 3-[2,4-bis[(3S)-3methylmorpholin-4-yl]pyrido[2,3-d]pyrimidin-7-yl]-N-methylbenzamide (AZD2014), 2methyl-2-[4-(3-methyl-2-oxo-8-quinolin-3-ylimidazo[4,5-c]quinolin-1yl)phenyl]propanenitrile (BEZ235), (Z)-but-2-enedioic acid;8-(6-methoxypyridin-3-yl)-3methyl-1-[4-piperazin-1-yl-3-(trifluoromethyl)phenyl]imidazo[4,5-c]quinolin-2-one (BGT226), Everolimus, 5-ethyl-3-[2-methyl-6-(1H-1,2,4-triazol-5-yl) pyridin-3yl]-7,8-dihydropyrazino[2,3-b]pyrazin-6-one (CC-115), 3-[6-(2-hydroxypropan-2 yl)pyridin-3-yl]-5-(4-methoxycyclohexyl)-7,8-dihydropyrazino[2,3-b]pyrazin-6-one (CC223), 8-[5-(2-hydroxypropan-2-yl) pyridin-3-yl]-1-[(2S)-2-methoxypropyl]-3methylimidazo[4,5-c]quinolin-2-one (LY3023414), P7170, 1-{(2R)-4-[2-(2Aminopyrimidin-5-yl)-6-(morpholin-4-yl)-9-(2,2,2-trifluoroethyl)-9H-purin-8-yl]-2 methylpiperazin-1-yl}ethan-1-one (DS-7423), (4-[(5Z)-4-amino-5-(7-methoxyindol-2 ylidene)-1H-imidazo[5,1-f][1,2,4]triazin-7-yl]cyclohexane-1-carboxylic acid) (OSI-027), 2-amino-8-[4-(2-hydroxyethoxy)cyclohexyl]-6-(6-methoxypyridin-3-yl)-4methylpyrido[2,3-d]pyrimidin-7-one (PF-04691502), 1-[4-[4-(dimethylamino) piperidine-1carbonyl]phenyl]-3-[4-(4,6-dimorpholin-4-yl-1,3,5-triazin-2-yl)phenyl]urea (PF-05212384), Temsirolimus, [6-(2-amino-1,3-benzoxazol-5-yl) imidazo[1,2-a]pyridin-3-yl]morpholin-4 ylmethanone (MLN1117), Ridaforolimus, Metformin, N-[4-[[3-(3,5dimethoxyanilino)quinoxalin-2-yl]sulfamoyl]phenyl]-3-methoxy-4-methylbenzamide (XL765), 2-amino-8-ethyl-4-methyl-6-(1H-pyrazol-5-yl)pyrido[2,3-d]pyrimidin-7-one (SAR245409), (3S)-4-[(1S)-1-carboxy-2-hydroxyethyl]amino]-3-[[2-[[(2S)-5 (diaminomethylidencamino)-2-[[4-oxo-4-[[4-(4-oxo-8-phenylchromen-2-yl) morpholin-4 ium-4-yl]methoxy]butanoyl]amino]pentanoyl]amino]acetyl]amino]-4-oxobutanoate (SF1126), 5-(8-methyl-2-morpholin-4-yl-9-propan-2-ylpurin-6-yl)pyrimidin-2-amine (VS5584), (2S)-1-[4-[[2-(2-aminopyrimidin-5-yl)-7-methyl-4-morpholin-4-ylthieno[3,2d]pyrimidin-6-yl]methyl]piperazin-1-yl]-2-hydroxypropan-1-one (GDC0980), or a compound of any of chemical formulae 1 to 5: 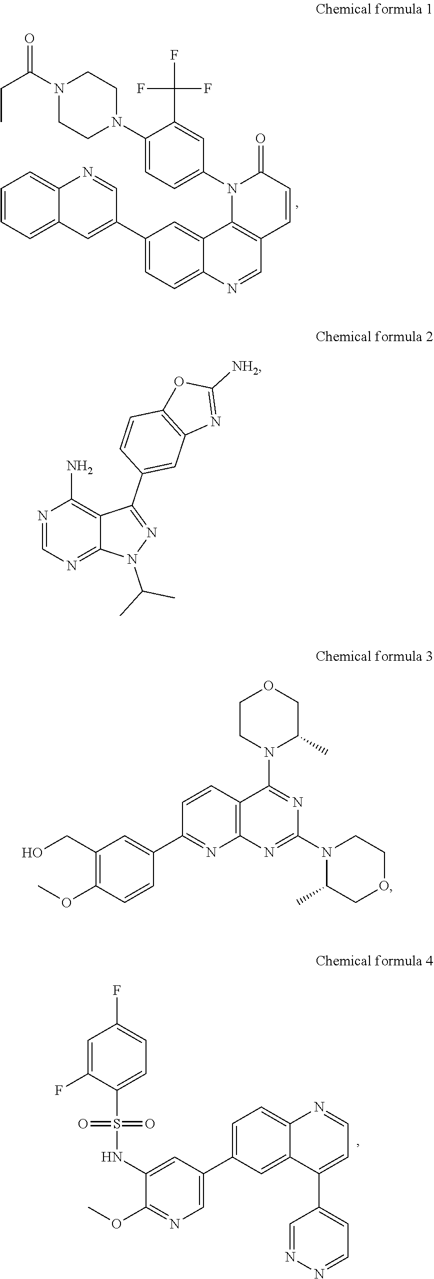 [SMILES: CCC(=O)N1CCN(c2ccc(-n3c(=O)ccc4cnc5ccc(-c6cnc7ccccc7c6)cc5c43)cc2C(F)(F)F)CC1 ; CC(C)n1nc(-c2ccc3oc(N)nc3c2)c2c(N)ncnc21 ; COc1ccc(-c2ccc3c(N4CCOC[C@@H]4C)nc(N4CCOC[C@@H]4C)nc3n2)cc1CO ; COc1ncc(-c2ccc3nccc(-c4ccnnc4)c3c2)cc1NS(=O)(=O)c1ccc(F)cc1F] 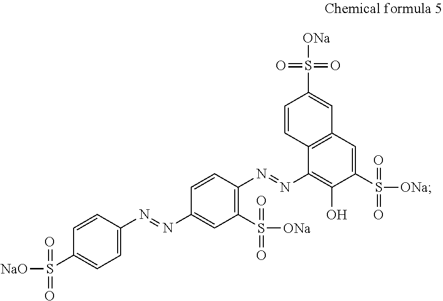
or a pharmaceutically-acceptable salt thereof.

4. A biomarker panel for diagnosing Focal Cortical Dysplasia (FCD) type II in a subject, wherein the biomarker panel comprises an agent detecting at least a brain somatic mutation in mTOR, TSC1, TSC2, AKT3, or PIK3CA, and wherein the subject is characterized by mTOR pathway hyperactivation and dysmorphic neurons in cerebral cortex.

5. The biomarker panel according to claim 4, wherein the agent detects at least one amino acid substitution or at least one nucleotide mutation encoding the amino acid substitution.

6. The biomarker panel according to claim 4, wherein the biomarker panel is applied to a brain tissue of the subject.

7. The biomarker panel according to claim 5, wherein the amino acid substitution is from Cytosine (C) at position 616 to Thymine (T) in an amino acid of SEQ ID NO: 2, an amino acid substitution of from arginine (R) at position 22 to tryptophan (W) in an amino acid of SEQ ID NO: 4, an amino acid substitution of from valine (V) at position 1547 to isoleucine (I) in an amino acid of SEQ ID NO: 6, an amino acid substitution of from arginine (R) at position 247 to histidine (H) in an amino acid of SEQ ID NO: 8, and an amino acid substitution of from aspartic acid (D) at position 1018 to asparagine (N) in an amino acid of SEQ ID NO: 10, or
the polynucleotide that is selected from the group consisting of a polynucleotide comprising a nucleotide mutation of Cytosine (C) at position 616 to Thymine (T) in an amino acid of SEQ ID NO: 1, nucleotide mutation of from Cytosine (C) at position 64 to Thymine (T) in an amino acid of SEQ ID NO: 3, nucleotide mutation of from Guanine (G) at position 4639 to Adenine (A) in an amino acid of SEQ ID NO: 5, nucleotide mutation of from Guanine (G) at position 740 to Adenine (A) in an amino acid of SEQ ID NO: 7, and nucleotide mutation of from Guanine (G) at position 3052 to Adenine (A) in an amino acid of SEQ ID NO: 9.
